(19) United States
(12) Patent Application Publication
(10) Pub. No.: US 2026/0199058 A1
(43) Pub. Date: Jul. 16, 2026

(54) Neural Network Techniques for Appliance Creation in Digital Oral Care

(71) Applicant: Solventum Intellectual Properties Company, Maplewood, MN (US)

(72) Inventors: Jonathan D. Gandrud, Woodbury, MN (US); Francis J.T. Yates, Dublin (IE); Seyed Amir Hossein Hosseini, Saint Paul, MN (US); Steve C. Demlow, Mendota Heights, MN (US); Michael B. Starr, Toomevara (IE)

(21) Appl. No.: 19/139,151

(22) PCT Filed: Dec. 14, 2023
(86) PCT No.: PCT/IB2023/062709

§ 371 Date: Jun. 13, 2025

Related U.S. Application Data
(60) Provisional application No. 63/432,627, filed on Dec. 14, 2022.
(60) Provisional application No. 63/461,629, filed on Apr. 25, 2023.

(51) Int. Cl. A61C 7/00; G16H 20/30; G16H 30/20

(52) U.S. Cl. CPC A61C 7/002; G16H 20/30; G16H 30/20; A61C 2007/004

(57) ABSTRACT

Systems and methods are disclosed for generating a three-dimensional (3D) representation of oral care data for use in oral care treatment. The systems and methods involve receiving an input 3D representation of a patient's dentition and encoding the 3D representation into a lower-dimensional first latent representation using a trained first machine learning (ML) module. Subsequently, a trained second ML module, comprising a trained transformer encoder model or a trained transformer decoder model, is executed to generate a second latent representation using the first latent representation. The second latent representation is then reconstructed into a 3D oral care representation (e.g., a tooth restoration design, an appliance component, a fixture model component, etc.) by a decoder. Finally, the processing circuitry outputs the reconstructed 3D representation of oral care data. These systems and methods enable efficient and accurate generation of oral care data, facilitating improved oral care appliance generation, treatment planning and analysis.

### RELATED DOCUMENTS

The entire disclosure of PCT Application No. PCT/IB2022/057373 is incorporated herein by reference. The entire disclosures of each of PCT Applications with Publication Nos. WO2022123402A1, WO2021240290A1, WO2020240351A1, WO2021245480A1, and WO2020026117A1 are incorporated herein by reference. The entire disclosure of each of the following Provisional U.S. Patent applications is incorporated herein by reference: 63/432,627; 63/366,492; 63/366,495; 63/352,850; 63/366,490; 63/366,494; 63/370,160; 63/366,507; 63/352,877; 63/366,514; 63/366,498; 63/366,514; and 63/264,914.

### TECHNICAL FIELD

This disclosure relates to configurations and training of machine learning models to improve the accuracy and data precision of 3D oral care representations to be used in dental or orthodontic treatments.

### SUMMARY

The present disclosure describes systems and techniques for training and using one or more machine learning models, such as neural networks to produce 3D oral care representations. Neural network-based techniques are described for the placement of oral care articles in relation to one or more 3D representations of teeth. Oral care articles which are to be placed may include: a dental restoration appliance component, oral care hardware (e.g., a lingual bracket, a labial bracket, an orthodontic attachment, a bite ramp, etc.), and the like. Furthermore, neural network-based techniques are described for the generation of the geometry and/or structure of oral care articles based, at least in part, on one or more 3D representations of teeth. Oral care articles which may be generated include: a dental restoration appliance component, a dental restoration tooth design, a crown, a veneer, and the like. Of the neural networks which may be trained to generate 3D oral care representations, transformers are an example of models that may enable improvements to data precision. Transformers may be trained to automatically generate 3D oral care representations, such as 3D meshes or 3D point clouds. Further examples of 3D oral care representations which may be generated include, but are not limited to: archforms, clear tray aligner (CTA) trimlines, and appliance components (e.g., such as generated components for use in creating a dental restoration appliance). In some instances, a transformer may be trained to generate 3D polylines (e.g., for archforms and CTA trimlines), or sets of control points (e.g., control points through which a spline can be fitted for an archform). Transformers may yield results which improve the data accuracy and data precision over prior techniques for 3D mesh/3D polyline generation, each of which may potentially comprise hundreds or even thousands of mesh elements, due to the capability of transformers to process long sequences of data. A neural network, such as a transformer, may be trained to predict an archform (e.g., taking dental arch and tooth data as input). An archform may take the form of a surface, a 3D mesh, a 3D polyline, or a set of control points (e.g., used to define a spline). Such an archform may, in some instances, be given as an input to a setups prediction machine learning model, such as a setups prediction neural network.

Techniques of this disclosure may train an encoder-decoder structure to generate (or modify) 3D oral care representations (e.g., tooth restoration designs, IPR cut surfaces, appliance components or others disclosed herein) which are suitable for oral care appliance generation. An encoder-decoder structure may comprise at least one encoder or at least one decoder. Non-limiting examples of an encoder-decoder structure include a 3D U-Net, a transformer, a pyramid encoder-decoder or an autoencoder, among others. Non-limiting examples of autoencoders include variational autoencoders, regularized autoencoders, masked autoencoders or capsule autoencoders. In some implementations, the generative techniques described herein may contain aspects derived from denoising diffusion models (e.g., a neural network which may be trained to iteratively denoise one or more 3D oral care representations—for use in appliance generation). In some implementations, the generative techniques described herein may train one or more neural networks to use mathematical operations associated with continuous normalizing flows (e.g., the use of a neural network which may be trained in one form and then be inverted for use in inference).

Techniques of this disclosure may be trained for the generation of a three-dimensional (3D) representation of oral care data which may be used in oral care treatment. An input 3D representation of a patient's dentition may undergo latent representation encoding (e.g., into a latent representation which has a lower order of dimensionality than the input data). A first machine learning (ML) module may be trained to perform the latent encoding. The first ML module may provide its output to a trained second ML module, which may contain one or more transformer encoders or one or more transformer decoders, which may generate a second latent representation based, at least in part, on the first latent representation. Furthermore, oral care arguments may be provided to the second ML module, to customize the outputs of the second ML module. The second latent representation may be reconstructed into a 3D oral care representation using a decoder, and the reconstructed representation may be outputted for use in oral care appliance generation. In some implementations, a trained latent representation modification module (LRMM) may be used to modify one or more aspects of the first latent space representation (e.g., in response to one or more oral care arguments). The modified first latent representation may be reconstructed into a representation which has been customized for use in treating a particular patient. The customized representation may be used in oral care appliance generation (e.g., a dental restoration appliance, an orthodontic appliance, etc.). The methods may generate a representation of an appliance component, a representation of an archform, a representation of the patient's dentition with at least one generated fixture model component, a representation of at least one tooth in a pre-restoration state, a representation of at least one tooth in a post-restoration state, or other representations described herein. The input 3D representation of the patient's dentition may comprise one or more mesh elements. Mesh element features may be computed for the mesh elements, and subsequently be provided to the first ML module (or the second ML module), to improve the accuracy of the resulting latent representation. In some implementations, the first ML module may contain one or more of an encoder, a U-Net, a pyramid encoder-decoder, 3D SWIN transformer, one or more convolutional layers, or one or more pooling layers. Either of both of the trained first ML module or the trained second ML module may, in some implementations, be trained according to a transfer learning paradigm. Likewise, either or both of the first ML module or the second ML module may be used to train, at least in part, one or more other ML models for use in digital oral care, according to a transfer learning paradigm. The methods may be deployed at a clinical context, and may be performed in near real-time during an encounter with a patient. The trained first ML module may, in some implementations, be configured to generate one or more hierarchical neural network features, which may be based at least in part on one or more aspects of at least one of the shape or the structure of the input 3D representation.

### BRIEF DESCRIPTION OF DRAWINGS

FIG. 1 shows a transformer which may be configured to generate orthodontic setups transforms.

FIG. 2 shows a method of augmenting training data for use in training machine learning (ML) models of this disclosure.

FIG. 3 shows a method of training a machine learning (ML) model to generate (or modify) a 3D representation of oral care data (e.g., oral care meshes).

FIG. 4 shows a method of using a trained ML model to generate (or modify) a 3D representation of oral care data (e.g., oral care meshes)

FIG. 5 shows a method of training an ML model to generate an archform.

FIG. 6 shows a method of training a reconstruction autoencoder which may be trained to reconstruction oral care meshes (e.g., archforms, teeth, tooth restoration designs, etc.).

FIG. 7 shows a method of using a trained reconstruction autoencoder to reconstruct oral care meshes (e.g., archforms, teeth, tooth restoration designs, etc.)

FIG. 8 shows a method of training a reconstruction autoencoder.

FIG. 9 shows a latent space where loss incorporates reconstruction loss but does not incorporate KL-Divergence loss.

FIG. 10 shows a latent space in which the loss includes both reconstruction loss and KL-divergence loss.

FIG. 11 shows a recursive inference (RI) model for 3D oral care representation generation (or modification).

FIG. 12 shows a method of using a trained ML model which contains one or more transformers to generate (or modify) a 3D representation of oral care data (e.g., a tooth restoration design, a fixture model component, an appliance component, etc.)

FIG. 13 shows a U-Net structure, which may be used to extract hierarchical features from a 3D representation.

FIG. 14 shows a pyramid encoder-decoder structure, which may be used to extract hierarchical features from a 3D representation.

FIG. 15 shows the fixture model component of interproximal webbing.

FIG. 16 shows the fixture model component of blockout.

FIG. 17 shows the fixture model component of digital pontic teeth.

FIG. 18 shows the fixture model component of interproximal reinforcement structures.

FIG. 19 shows the fixture model component of a gingival ridge structure.

FIG. 20 shows a visualization of reconstruction error for a tooth.

FIG. 21 shows a 3D representation of an archform which may be generated (or modified) using methods of this disclosure.

FIG. 22 shows a method of training a latent representation modification module (LRMM).

FIG. 23 shows a method of using a fully trained LRMM.

### DETAILED DESCRIPTION

The machine learning techniques described herein may receive a variety of input data, as described herein, including tooth meshes for one or both dental arches of the patient. The tooth data may be presented in the form of 3D representations, such as meshes, point clouds, or voxelized geometries. These data may be preprocessed, for example, by arranging the constituent mesh elements into lists and computing an optional mesh element feature vector for each mesh element. Such vectors may impart valuable information about the shape and/or structure of an oral care mesh to the machine learning models described herein. Additional inputs may be received as the input to the machine learning models described herein, such as one or more oral care metrics. Oral care metrics may be used for measuring one or more physical aspects of an oral care mesh (e.g., physical relationships within a tooth or between different teeth). In some instances, an oral care metric may be computed for either or both of a malocclusion oral care mesh example and/or a ground truth oral care mesh example which is then used in the training of the machine learning models described herein. The metric value may be received as the input to the machine learning models described herein, as a way of training that model or those models to encode a distribution of such a metric over the several examples of the training dataset. During training, the network may then receive metric value(s) as input, to assist in training the network to link that inputted metric value to the physical aspects of the ground truth oral care mesh which is used in loss calculation. Such a loss calculation may quantify the difference between a prediction and a ground truth example (e.g., between a predicted oral care mesh and a ground truth oral care mesh). By providing the network with metric value(s), the neural network techniques of this disclosure may, through the course of loss calculation and subsequent backpropagation, learn train the neural network to encode a distribution of a given metric. In deployment, one or more oral care parameters (procedure parameters or restoration design parameters) may be defined to specify one or more aspects of an intended oral care mesh, which is to be generated using the machine learning models described herein, which have been trained for that purpose.

One or more oral care arguments may be defined to specify one or more aspects of an intended 3D oral care representation (e.g., a 3D mesh, a polyline, a 3D point cloud or a voxelized geometry), which is to be generated using the machine learning models described herein (e.g., 3D representation generation models using transformers) that have been trained for that purpose. In some implementations, oral care arguments may be defined to specify one or more aspects of a customized vector, matrix or any other numerical representation (e.g., to describe 3D oral care representations such as control points for a spline, an archform, a transform to place a tooth or appliance component relative to another 3D oral care representation, or a coordinate system), which is to be generated using the machine learning models described herein (e.g., 3D representation generation models using transformers) that have been trained for that purpose. A customized vector, matrix or other numerical representation may describe a 3D oral care representation which conforms to the intended outcome of the treatment of the patient. Oral care arguments may include oral care metrics or oral care parameters, among others. Oral care arguments may specify one or more aspects of an oral care procedure—such as orthodontic setups prediction or restoration design generation, among others. In some implementations, one or more oral care parameters may be defined which correspond to respective oral care metrics. Oral care arguments can be provided as the input to the machine learning models described herein and be taken as an instruction to that module to generate an oral care mesh with the specified customization, to place an oral care mesh for the generation of an orthodontic setup (or appliance), to segment an oral care mesh, or to clean up an oral care mesh, to generate or modify a 3D representation of oral care data, to name a few examples. This interplay between oral care metrics and oral care parameters may also apply to the training and deployment of other predictive models in oral care as well.

The predictive models of the present disclosure may, in some implementations, may produce more accurate results by the incorporation of one or more of the following inputs: archform information V, interproximal reduction (IPR) information U, tooth dimension information P, tooth gap information Q, latent capsule representations of oral care meshes T, latent vector representations of oral care meshes A, procedure parameters K (which may describe a clinician's intended treatment of the patient), doctor preferences L (which may describe the typical procedure parameters chosen by a doctor), flags regarding tooth status M (such as for fixed or pinned teeth), tooth position information N, tooth orientation information O, tooth name/dental notation R, oral care metrics S (comprising of at least one of oral care metrics and restoration design metrics).

Systems of this disclosure may, in some instances, be deployed at a clinical context (such as a dental or orthodontic office) for use by clinicians (e.g., doctors, dentists, orthodontists, nurses, hygienists, oral care technicians). Such systems which are deployed at a clinical context may enable clinicians to process oral care data (such as dental scans) in the clinic environment, or in some instances, in a “chairside” context (where the patient is present in the clinical environment). A non-limiting list of examples of techniques may include: segmentation, mesh cleanup, coordinate system prediction, CTA trimline generation, restoration design generation, appliance component generation or placement or assembly, generation of other oral care meshes, the validation of oral care meshes, setups prediction, removal of hardware from tooth meshes, hardware placement on teeth, imputation of missing values, clustering on oral care data, oral care mesh classification, setups comparison, metrics calculation, or metrics visualization. The execution of these techniques may, in some instances, enable patient data to be processed, analyzed and used in appliance generation by the clinician before the patient leaves the clinical environment (which may facilitate treatment planning because feedback may be received from the patient during the treatment planning process).

Systems of this disclosure may automate operations in digital orthodontics (e.g., setups prediction, hardware placement, setups comparison), in digital dentistry (e.g., restoration design generation) or in combinations thereof. Some techniques may apply to either or both of digital orthodontics and digital dentistry. A non-limiting list of examples is as follows: segmentation, mesh cleanup, coordinate system prediction, oral care mesh validation, imputation of oral care parameters, oral care mesh generation or modification (e.g., using autoencoders, transformers, continuous normalizing flows or denoising diffusion models), metrics visualization, appliance component placement or appliance component generation or the like. In some instances, systems of this disclosure may enable a clinician or technician to process oral care data (such as scanned dental arches). In addition to segmentation, mesh cleanup, coordinate system prediction or validation operations, the systems of this disclosure may enable orthodontic treatment planning, which may involve setups prediction as at least one operation. Systems of this disclosure may also enable restoration design generation, where one or more restored tooth designs are generated and processed in the course of creating oral care appliances. Systems of this disclosure may enable either or both of orthodontic or dental treatment planning, or may enable automation steps in the generation of either or both of orthodontic or dental appliances. Some appliances may enable both of dental and orthodontic treatment, while other appliances may enable one or the other.

Aspects of the present disclosure can provide a technical solution to the technical problem of generating, using 3D representations of a patient's dentition (and/or appliance components or fixture model components) and/or transformer neural networks, 3D oral care representations for use in oral care appliance generation. In particular, by practicing techniques disclosed herein computing systems specifically adapted to perform 3D oral care representation generation for use in generating oral care appliances are improved. For example, aspects of the present disclosure improve the performance of a computing system having a 3D representation of the patient's dentition by reducing the consumption of computing resources. In particular, aspects of the present disclosure reduce computing resource consumption by decimating 3D representations of the patient's dentition (e.g., reducing the counts of mesh elements used to describe aspects of the patient's dentition) so that computing resources are not unnecessarily wasted by processing excess quantities of mesh elements. Additionally, decimating the meshes does not reduce the overall predictive accuracy of the computing system (and indeed may actually improve predictions because the input provided to the ML model after decimation is a more accurate (or better) representation of the patient's dentition). For example, noise or other artifacts which are unimportant (and which may reduce the accuracy of the predictive models) are removed. That is, aspects of the present disclosure for more efficient allocation of computing resources and in a way that improves the accuracy of the underlying system.

Furthermore, aspects of the present disclosure may need to be executed in a time-constrained manner, such as when an oral care appliance must be generated for a patient immediately after intraoral scanning (e.g., while the patient waits in the clinician's office). As such, aspects of the present disclosure are necessarily rooted in the underlying computer technology of 3D oral care representation generation using transformer neural networks and cannot be performed by a human, even with the aid of pen and paper. For instance, implementations of the present disclosure must be capable of: 1) storing thousands or millions of mesh elements of the patient's dentition in a manner that can be processed by a computer processor; 2) performing calculation on thousands or millions of mesh elements, e.g., to quantify aspects of the shape and or/structure of an individual tooth in the 3D representation of the patient's dentition; and 3) generating, based on a transformer neural network, 3D oral care representations for use in oral care appliance generation (e.g., orthodontic aligner trays, dental restoration appliances, indirect bonding trays for orthodontic treatment, or the like), and do so during the course of a short office visit.

This disclosure pertains to digital oral care, which encompasses the fields of digital dentistry and digital orthodontics. This disclosure generally describes methods of processing three-dimensional (3D) representations of oral care data. It should be understood, without loss of generality, that there are various types of 3D representations. One type of 3D representation is 3D geometry. A 3D representation may include, be, or be part of one or more of a 3D polygon mesh, a 3D point cloud (e.g., such as derived from a 3D mesh), a 3D voxelized representation (e.g., a collection of voxels—for sparse processing), or 3D representations which are described by mathematical equations. Although the term “mesh” is used frequently throughout this disclosure, the term should be understood, in some implementations, to be interchangeable with other types of 3D representations. A 3D representation may describe elements of the 3D geometry and/or 3D structure of an object.

Dental arches S1, S2, S3 and S4 all contain the exact same tooth meshes, but those tooth meshes are transformed differently, according to the following description. A first arch S1 includes a set of tooth meshes arranged (e.g., using transforms) in their positions in the mouth, where the teeth are in the mal positions and orientations. A second arch S2 includes the same set of tooth meshes from S1 arranged (e.g., using transforms) in their positions in the mouth, where the teeth are in the ground truth setup positions and orientations. A third arch S3 includes the same meshes as S1 and S2, which are arranged (e.g., using transforms) in their positions in the mouth, where the teeth are in the predicted final setup poses (e.g., as predicted by one or more of the techniques of this disclosure). S4 is a counterpart to S3, where the teeth are in the poses corresponding to one of the several intermediate stages of orthodontic treatment with clear tray aligners.

It should be understood, without the loss of generality, that the techniques of this disclosure which apply to final setups are also applicable to intermediate staging in orthodontic treatment, particularly geometric deep learning (GDL) Setups, reinforcement learning (RL) Setups, variational autoencoder (VAE) Setups, Capsule Setups, multilayer perceptron (MLP) Setups, Diffusion Setups, pose transfer (PT) Setups, Similarity Setups, force directed graphs (FDG) Setups, Transformer Setups, Setups Comparison, or Setups Classification. The Metrics Visualization aspects of this disclosure may also be configured to visualize data from both final setups and intermediate stages. MLP Setups, VAE Setups and Capsule Setups each fall within the scope of Autoencoder Setups. Some implementations of MLP Setups may fall within the Scope of Transformer Setups. Representation Setups refers to any of MLP Setups, VAE Setups, Capsule Setups and any other setups prediction machine learning model which uses an autoencoder to create the representation for at least one tooth.

Each of the setups prediction techniques of this disclosure is applicable to the fabrication of clear tray aligners and/or indirect bonding trays. The setups predictions techniques may also be applicable to other products that involve final teeth poses, also. A pose may comprise a position (or location) and a rotation (or orientation).

A 3D mesh is a data structure which may describe the geometry or shape of an object related to oral care, including but not limited to a tooth, a hardware element, or a patient's gum tissue. A 3D mesh may include one or more mesh elements such as one or more of vertices, edges, faces and combinations thereof. In some implementations, mesh elements may include voxels, such as in the context of sparse mesh processing operations. Various spatial and structural features may be computed for these mesh elements and be provided to the predictive models of this disclosure, with the predictive models of this disclosure providing the technical advantage of improving data precision in the form of the models of this disclosure outputting more accurate predictions.

A patient's dentition may include one or more 3D representations of the patient's teeth (e.g., and/or associated transforms), gums and/or other oral anatomy. An orthodontic metric (OM) may, in some implementations, quantify the relative positions and/or orientations of at least one 3D representation of a tooth relative to at least one other 3D representation of a tooth. A restoration design metric (RDM) may, in some implementations, quantify at least one aspect of the structure and/or shape of a 3D representation of a tooth. An orthodontic landmark (OL) may, in some implementations, locate one or more points or other structural regions of interest on a 3D representation of a tooth. An OL may, in some implementations, be used in the generation of an orthodontic or dental appliance, such as a clear tray aligner or a dental restoration appliance. A mesh element may, in some implementations, comprise at least one constituent element of a 3D representation of oral care data. For example, in the case of a tooth that is represented by a 3D mesh, mesh elements may include at least: vertices, edges, faces and voxels. A mesh element feature may, in some implementations, quantify some aspect of a 3D representation in proximity to or in relation with one or more mesh elements, as described elsewhere in this disclosure. Orthodontic procedure parameters (OPP) may, in some implementations, specify at least one value which defines at least one aspect of planned orthodontic treatment for the patient (e.g., specifying desired target attributes of a final setup in final setups prediction). Orthodontic Doctor preferences (ODP) may, in some implementations, specify at least one typical value for an OPP, which may, in some instances, be derived from past cases which have been treated by one or more oral care practitioners. Restoration Design Parameters (RDP) may, in some implementations, specify at least one value which defines at least one aspect of planned dental restoration treatment for the patient (e.g., specifying desired target attributes of a tooth which is to undergo treatment with a dental restoration appliance). Doctor Restoration Design Preferences (DRDP) may, in some implementations, specify at least one typical value for an RDP, which may, in some instances, be derived from past cases which have been treated by one or more oral care practitioners. 3D oral care representations may include, but are not limited to: 1) a set of mesh element labels which may be applied to the 3D mesh elements of teeth/gums/hardware/appliance meshes (or point clouds) in the course of mesh segmentation or mesh cleanup; 2) 3D representation(s) for one or more teeth/gums/hardware/appliances for which shapes have been modified (e.g., trimmed, distorted, or filled-in) in the course of mesh segmentation or mesh cleanup; 3) one or more coordinate systems (e.g., describing one, two, three or more coordinate axes) for a single tooth or a group of teeth (such as a full arch—as with the LDE coordinate system); 4) 3D representation(s) for one or more teeth for which shapes have been modified or otherwise made suitable for use in dental restoration; 5) 3D representation(s) for one or more dental restoration appliance components; 6) one or more transforms to be applied to one or more of: dental restoration appliance library component placement relative to one or more teeth, a tooth to be placed for an orthodontic setup (either final setup or intermediate stage), a hardware element to be placed relative to one or more teeth or the like; 7) an orthodontic setup; 8) a 3D representation of a hardware element (such as facial bracket, lingual bracket, orthodontic attachment, button, hook, bite ramp, etc.) to be placed relative to one or more teeth, etc.; 8) a 3D representation of a bonding pad for a hardware element (which may be generated for a specific tooth by outlining a perimeter on the tooth, specifying a thickness to form a shell, and then subtracting-out the tooth via a Boolean operation); 9) 3D representation of a clear tray aligner (CTA); 10) the location or shape of a CTA trimline (e.g., described as either a mesh or polyline); 11) archform that describes the contours or layout of an arch of teeth (e.g., described as a 3D polyline or as a 3D mesh or surface), which may follow the incisal edges one or more teeth, which may follow the facial surfaces of one or more teeth, which may in some implementations correspond to the maloccluded arch and in other implementations correspond to the final setup arch (the effects of malocclusion on the shape of the archform may be diminished by smoothing or averaging of the shape of the archform), which may be described by one or more control points and/or a spline; 12) 3D representation of a fixture models (e.g., depictions of teeth and gums for use in thermoforming clear tray aligners, or depictions of teeth/gums/hardware for use in thermoforming indirect bonding trays); 13) one or more latent space vectors (or latent capsules) produced by the 3D encoder stage of a 3D autoencoder which has been trained on the reconstruction of oral care meshes (e.g., a variational autoencoder which has been trained for tooth reconstruction); 14) one or more oral care metrics values (e.g., such as orthodontic metrics or restoration design generation metrics) for one or more teeth; 15) one or more landmarks (e.g., 3D points) which describe the shapes and/or geometrical attributes of one or more teeth, other dentition structures or hardware structures (e.g., to be used in orthodontic setups creation or restoration appliance component generation or placement); 16) 3D representation created by scanning (e.g., optically scanning, CT scanning or MRI scanning) a 3D printed part corresponding to one or more teeth/gums/hardware/appliances (e.g., a scanned fixture model); 17) 3D printed aligners (including optionally local thickness, reinforcing rib geometry, flap positioning, or the like) 18) 3D representation of the patient's dentition that was captured chairside by a clinician or medical practitioner (e.g., in a context where the 3D representation is validated chairside, before the patient leaves the clinic, so that errors can be detected and re-scans performed as necessary); 19) dental restoration tooth design (e.g., for veneers, crowns, bridges or dental restoration appliances); 20) 3D representations of one or more teeth for use in digital oral care treatment; 21) other 3D printed parts pertaining to oral care procedures or other fields; 22) IPR cut surfaces—such as an IPR cut plane which may define a portion of enamel to be removed from a tooth; 23) one or more orthodontic setups transforms associated with one or more IPR cut surfaces; 24) a (digital) pontic tooth design which may fill at least a portion of the space between teeth to allow room in an orthodontic setup for an erupting tooth to later emerge from the gums; or 25) a component of a fixture model (e.g., comprising fixture model components such as interproximal webbing, block-out, bite locks, bite ramps, interproximal reinforcement, gingival ridge structures, torque points, power ridges, pontic tooth or dimples, among others).

Techniques of this disclosure may require a training dataset of hundreds or thousands of cohort patient cases, to ensure that the neural network is able to encode the distribution of patient cases which are likely to be encountered in clinical treatment. A cohort patient case may include a set of tooth crown meshes, a set of tooth root meshes, or a data file containing attributes of the case (e.g., a JSON file). A typical example of a cohort patient case may contain up to 32 crown meshes (e.g., which may each contain tens of thousands of vertices or tens of thousands of faces), up to 32 root meshes (e.g., which may each contain tens of thousands of vertices or tens of thousands of faces), multiple gingiva mesh (e.g., which may each contain tens of thousands of vertices or tens of thousands of faces) or one or more JSON files which may each contain tens of thousands of values (e.g., objects, arrays, strings, real values, Boolean values or Null values).

The techniques of this disclosure may be advantageously combined. For example, the Setups Comparison tool may be used to compare the output of the GDL Setups model against ground truth data, compare the output of the RL Setups model against ground truth data, compare the output of the VAE Setups model against ground truth data and compare the output of the MLP Setups model against ground truth data. With each of these setups prediction models compared against ground truth data, it may be possible to determine which model gives the best performance on a certain dataset or within a given problem domain. Furthermore, the Metrics Visualization tool can enable a global view of the final setups and intermediate stages produced by one or more of the setups prediction models, with the advantage of enabling the selection of the best setups prediction model. The Metrics Visualization tool, furthermore, enables the computation of metrics which have a global scope over a set of intermediate stages. These global metrics may, in some implementations, be consumed as inputs to the neural networks for predicting setups (e.g., GDL Setups, RL Setups, VAE Setups, Capsule Setups, MLP Setups, Diffusion Setups, PT Setups, Similarity Setups, among others). The global metrics may also be provided to FDG Setups. The local metrics from this disclosure (i.e., a local metric is a metric which may be computed for one stage or setup of treatment, rather than over several stages or setups) may, in some implementations, be consumed by the neural networks herein for predicting setups, with the advantage of improving predictive results. The metrics described in this disclosure may, in some implementations, be visualized using the Metric Visualization tool.

The VAE and MAE models for mesh element labelling and mesh in-filling can be advantageously combined with the setups prediction neural networks, for the purpose of mesh cleanup ahead of or during the prediction process. In some implementations, the VAE for mesh element labelling may be used to flag mesh elements for further processing, such as metrics calculation, removal, or modification. In some instances, such flagged mesh elements may be provided as inputs to a setups prediction neural network, to inform that neural network about important mesh features, attributes or geometries, with the advantage of improving the performance of the resulting setups prediction model. In some implementations, mesh in-filling may cause the geometry of a tooth to become more nearly complete, enabling the better functioning of a setups prediction model (i.e., improved correctness of prediction on account of better-formed geometry). In some instances, a neural network to classify a setup (i.e., the Setups Classifier) may aid in the functioning of a setups prediction neural network, because the setups classifier tells that setups prediction neural network when the predicted setup is acceptable for use and can be provided to a method for aligner tray generation. A Setups Classifier (e.g., GDL Setups, RL Setups, VAE Setups, Capsule Setups, MLP Setups, Diffusion Setups, PT Setups, Similarity Setups and FDG Setups, among others) may aid in the generation of final setups and also in the generation of intermediate stages. Furthermore, a Setups Classifier neural network may be combined with the Metrics Visualization tool. In other implementations, a Setups Classification neural network may be combined with the Setups Comparison tool (e.g., the Setup Comparison tool may output an indication of how a setup produced in part by the Setups Classifier compares to a setup produced by another setups prediction method). In some implementations, the VAE for mesh element labelling may identify one or more mesh elements for use in a metrics calculation. The resulting metrics outputs may be visualized by the Metrics Visualization tool.

In some examples, the Setups Classifier neural network may aid in the setups prediction technique described in U.S. Patent Application No. US20210259808A1 (which is incorporated herein by reference in its entirety) or the setups prediction technique described in PCT Application with Publication No. WO2021245480A1 (which is incorporated herein by reference in its entirety) or in PCT Application No. PCT/IB2022/057373 (which is incorporated herein by reference in its entirety). The Setups Classifier would help one or more of those techniques to know when the predicted final setup is most nearly correct. In some instances, the Setups Classifier neural network may output an indication of how far away from final setup a given setup is (i.e., a progress indicator).

In some implementations, the latent space embedding vector(s) from the reconstruction VAE can be concatenated with the inputs to the setups prediction neural network described in WO2021245480A1. The latent space vectors can also be incorporated as inputs to the other setups prediction models: GDL Setups, RL Setups, VAE Setups, Capsule Setups, MLP Setups and Diffusion Setups, among others. The advantage is to impart the reconstruction characteristics (e.g., latent vector dimensions of a tooth mesh) to that neural network, hence improving the generated setups prediction.

In some examples, the various setups prediction neural networks of this disclosure may work together to produce the setups required for orthodontic treatment. For example, the GDL Setups model may produce a final setup, and the RL Setups model may use that final setup as input to produce a series of intermediate stages setups. Alternatively, the VAE Setups model (or the MLP Setups model) may create a final setup which may be used by an RL Setups model to produce a series of intermediate stages setups. In some implementations, a setup prediction may be produced by one setups prediction neural network, and then taken as input to another setups prediction neural network for further improvements and adjustments to be made. In some implementations, such improvements may be performed in iterative fashion.

In some implementations, a setups validation model, such as the model disclosed in US Provisional Application No. U.S. 63/366,495, may be involved in this iterative setups prediction loop. First a setup may be generated (e.g., using a model trained for setups prediction, such as GDL Setups, RL Setups, VAE Setups, Capsule Setups, MLP Setups, Diffusion Setups, PT Setups, Similarity Setups and FDG Setups, among others), then the setup undergoes validation. If the setup passes validation, the setup may be outputted for use. If the setup fails validation, the setup may be sent back to one or more of the setups prediction models for corrections, improvements and/or adjustments. In some instances, the setups validation model may output an indication of what is wrong with the setup, enabling the setups generation model to make an improved version upon the next iteration. The process iterates until done.

Generally speaking, in some implementations, two or more of the following techniques of the present disclosure may be combined in the course of orthodontic and/or dental treatment: GDL Setups, Setups Classification, Reinforcement Learning (RL) Setups, Setups Comparison, Autoencoder Setups (VAE Setups or Capsule Setups), VAE Mesh Element Labeling, Masked Autoencoder (MAE) Mesh In-filling, Multi-Layer Perceptron (MLP) Setups, Metrics Visualization, Imputation of Missing Oral Care Parameters Values, Tooth Classification Using Latent Vector, FDG Setups, Pose Transfer Setups, Restoration Design Metrics Calculation, Neural Network Techniques for Dental Restoration and/or Orthodontics (e.g., 3D Oral Care Representation Generation or Modification Using Transformers), Landmark-based (LB) Setups, Diffusion Setups, Imputation of Tooth Movement Procedures, Capsule Autoencoder Segmentation, Diffusion Segmentation, Similarity Setups, Validation of Oral Care Representations (e.g., using autoencoders), Coordinate System Prediction, Restoration Design Generation, or 3D Oral Care Representation Generation (or Modification) Using Diffusion Models.

In some instances, coordinate system prediction may be used in combination with techniques of this disclosure. Pose transfer techniques may be trained for coordinate system prediction, in the form of predicting a transform for a tooth. Reinforcement learning techniques may be trained for coordinate system prediction, in the form of predicting a transform for a tooth.

In some instances, tooth shape-based inputs may be provided to a neural network for setups predictions. In other instances, non-shape-based inputs can be used, such as a tooth name or designation, as it pertains to dental notation. In some implementations, a vector R of flags may be provided to the neural network, where a ‘1’ value indicates that the tooth is present and a ‘0’ value indicates that the tooth is absent from the patient case (though other values are possible). The vector R may comprise a 1-hot vector, where each element in the vector corresponds to a tooth type, name, or designation. Identifying information about a tooth (e.g., the tooth's name) can be provided to the predictive neural networks of this disclosure, with the advantage of enabling the neural network to become trained to handle different teeth in tooth-specific ways. For example, the setups prediction model may learn to make setups transformations predictions for a specific tooth designation (e.g., upper right central incisor, or lower left cuspid, etc.). In the case of the mesh cleanup autoencoders (either for labelling mesh element or for in-filling missing mesh data), the autoencoder may be trained to provide specialized treatment to a tooth according to that tooth's designation, in this manner. In the case of a setups classification neural network, a listing of tooth name(s) present in the patient's arch may better enable the neural network to output an accurate determination of setup classification, because tooth designation is a valuable input to training such a neural network. Tooth designation/name may be defined, for example, according to the Universal Numbering System, Palmer System, or the FDI World Dental Federation notation (ISO 3950).

In one example, where all except the (up to four) wisdom teeth are present in the case, a vector R may be defined as an optional input to the setups prediction neural networks of this disclosure, where there is a 0 in the vector element corresponding to each of the wisdom teeth, and a 1 in the elements corresponding to the following teeth: UR7, UR6, UR5, UR4, UR3, UR2, UR1, UL1, UL2, UL3, UL4, UL5, UL6, UL7, LL7, LL6, LL5, LL4, LL3, LL2, LL1, LR1, LR2, LR3, LR4, LR5, LR6, LR7

In some instances, the position of the tooth tip may be provided to a neural network for setups predictions. In other instances, one or more vectors S of the orthodontic metrics described elsewhere in this disclosure may be provided to a neural network for setups predictions. The advantage is an improved capacity for the network to become trained to understand the state of a maloccluded setup and therefore be able to predict a more accurate final setup or intermediate stage.

In some implementations, the neural networks may take as input one or more indications of interproximal reduction (IPR) U, which may indicate the amount of enamel that is to be removed from a tooth during the course orthodontic treatment (either mesially or distally). In some implementations, IPR information (e.g., quantity of IPR that is to be performed on one or more teeth, as measured in millimeters, or one or more binary flags to indicate whether or not IPR is to be performed on each tooth identified by flagging) may be concatenated with a latent vector A which is produced by a VAE or a latent capsule T autoencoder. The vector(s) and/or capsule(s) resulting from such a concatenation may be provided to one or more of the neural networks of the present disclosure, with the technical improvement or added advantage of enabling that predictive neural network to account for IPR. IPR is especially relevant to setups prediction methods, which may determine the positions and poses of teeth at the end of treatment or during one or more stages during treatment. It is important to account for the amount of enamel that is to be removed ahead of predicted tooth movements.

In some implementations, one or more procedure parameters K and/or doctor preferences vectors L may be introduced to a setups prediction model. In some implementations, one or more optional vectors or values of tooth position N (e.g., XYZ coordinates, in either tooth local or global coordinates), tooth orientation O (e.g., pose, such as in transformation matrices or quaternions, Euler angles or other forms described herein), dimensions of teeth P (e.g., length, width, height, circumference, diameter, diagonal measure, volume—any of which dimensions may be normalized in comparison to another tooth or teeth), distance between adjacent teeth Q. These “dimensions of teeth P” may in some instances be used to describe the intended dimensions of a tooth for dental restoration design generation.

In some implementations, tooth dimensions P such as length, width, height, or circumference may be measured inside a plane, such as the plane that intersects the centroid of the tooth, or the plane that intersects a center point that is located midway between the centroid and either the incisal-most extent or the gingival-most extent of the tooth. The tooth dimension of height may be measured as the distance from gums to incisal edge. The tooth dimension of width may be measured as the distance from the mesial extent to the distal extent of the tooth. In some implementations, the circularity or roundness of the tooth cross-section may be measured and included in the vector P. Circularity or roundness may be defined as the ratio of the radii of inscribed and circumscribed circles.

The distance Q between adjacent teeth can be implemented in different ways (and computed using different distance definitions, such as Euclidean or geodesic). In some implementations, a distance Q1 may be measured as an averaged distance between the mesh elements of two adjacent teeth. In some implementations, a distance Q2 may be measured as the distance between the centers or centroids of two adjacent teeth. In some implementations, a distance Q3 may be measured between the mesh elements of closest approach between two adjacent teeth. In some implementations, a distance Q4 may be measured between the cusp tips of two adjacent teeth. Teeth may, in some implementations, be considered adjacent within an arch. Teeth may, in some implementations, also be considered adjacent between opposing arches. In some implementations, any of Q1, Q2, Q3 and Q4 may be divided by a term for the purpose of normalizing the resulting value of Q. In some implementations, the normalizing term may involve one or more of: the volume of a tooth, the count of mesh elements in a tooth, the surface area of a tooth, the cross-sectional area of a tooth (e.g., as projected into the XY plane), or some other term related to tooth size.

Other information about the patient's dentition or treatment needs (or related parameters) may be concatenated with the other input vectors to one or more of MLP, GAN, generator, encoder structure, decoder structure, transformer, VAE, conditional VAE, regularized VAE, 3D U-Net, capsule autoencoder, diffusion model, and/or any of the neural networks models listed elsewhere in this disclosure.

The vector M may contain flags which apply to one or more teeth. In some implementations, M contains at least one flag for each tooth to indicate whether the tooth is pinned. In some implementations, M contains at least one flag for each tooth to indicate whether the tooth is fixed. In some implementations, M contains at least one flag for each tooth to indicate whether the tooth is pontic. Other and additional flags are possible for teeth, as are combinations of fixed, pinned and pontic flags. A flag that is set to a value that indicates that a tooth should be fixed is a signal to the network that the tooth should not move over the course of treatment. In some implementations, the neural network loss function may be designed to be penalized for any movement in the indicated teeth (and in some particular cases, may be heavily penalized). A flag to indicate that a tooth is pontic informs the network that the tooth gap is to be maintained, although that gap is allowed to move. In some cases, M may contain a flag indicating that a tooth is missing. In some implementations, the presence of one or more fixed teeth in an arch may aid in setups prediction, because the one or more fixed teeth may provide an anchor for the poses of the other teeth in the arch (i.e., may provide a fixed reference for the pose transformations of one or more of the other teeth in the arch). In some implementations, one or more teeth may be intentionally fixed, so as to provide an anchor against which the other teeth may be positioned. In some implementations, a 3D representation (such as a mesh) which corresponds to the gums may be introduced, to provide a reference point against which teeth can be moved.

Without the loss of generality, one or more of the optional input vectors K, L, M, N, O, P, Q, R, S, U and V described elsewhere in this disclosure may also be provided to the input or into an intermediate layer of one or more of the predictive models of this disclosure. In particular, these optional vectors may be provided to the MLP Setups, GDL Setups, RL Setups, VAE Setups, Capsule Setups and/or Diffusion Setups, with the advantage of enabling the respective model to generate setups which better meet the orthodontic treatment needs of the patient. In some implementations, such inputs may be introduced, for example, by being concatenated with one or more latent vectors A which are also provided to one or more of the predictive models of this disclosure. In some implementations, such inputs may be introduced, for example, by being concatenated with one or more latent capsules T which are also provided to one or more of the predictive models of this disclosure.

In some implementations, one or more of K, L, M, N, O, P, Q, R, S, U and V may be introduced to the neural network (e.g., MLP or Transformer) directly in a hidden layer of the network. In some instances, one or more of K, L, M, N, O, P, Q, R, S, U and V may be introduced directly into the internal processing of an encoder structure.

In some implementations, a setups prediction model (such as GDL Setups, RL Setups, VAE Setups, Capsule Setups, MLP Setups, PT Setups, Similarity Setups and Diffusion Setups) may take as input one or more latent vectors A which correspond to one or more input oral care meshes (e.g., such as tooth meshes). In some implementations, a setups prediction model (such as GDL Setups, RL Setups, VAE Setups, Capsule Setups, MLP Setups and Diffusion Setups) may take as input one or more latent capsules T which correspond to one or more input oral care meshes (e.g., such as tooth meshes). In some implementations, a setups prediction method may take as input both of A and T.

Various loss calculation techniques are generally applicable to the techniques of this disclosure (e.g., GDL Setups, RL Setups, VAE Setups, Capsule Setups, MLP Setups, Diffusion Setups, PT Setups, Similarity Setups, Setups Classification, Tooth Classification, VAE Mesh Element Labelling, MAE Mesh In-Filling and the imputation of procedure parameters).

These losses include L1 loss, L2 loss, mean squared error (MSE) loss, cross entropy loss, among others. Losses may be computed and used in the training of neural networks, such as multi-layer perceptron's (MLP), U-Net structures, generators, and discriminators (e.g., for GANs), autoencoders, variational autoencoders, regularized autoencoders, masked autoencoders, transformer structures, or the like. Some implementations may use either triplet loss or contrastive loss, for example, in the learning of sequences.

Losses may also be used to train encoder structures and decoder structures. A KL-Divergence loss may be used, at least in part, to train one or more of the neural networks of the present disclosure, such as a mesh reconstruction autoencoder or the generator of GDL Setups, which the advantage of imparting Gaussian behavior to the optimization space. This Gaussian behavior may enable a reconstruction autoencoder to produce a better reconstruction (e.g., when a latent vector representation is modified and that modified latent vector is reconstructed using a decoder, the resulting reconstruction is more likely to be a valid instance of the inputted representation). There are other techniques for computing losses which may be described elsewhere in this disclosure. Such losses may be based on quantifying the difference between two or more 3D representations.

MSE loss calculation may involve the calculation of an average squared distance between two sets, vectors or datasets. MSE may be generally minimized. MSE may be applicable to a regression problem, where the prediction generated by the neural network or other machine learning model may be a real number. In some implementations, a neural network may be equipped with one or more linear activation units on the output to generate an MSE prediction. Mean absolute error (MAE) loss and mean absolute percentage error (MAPE) loss can also be used in accordance with the techniques of this disclosure.

Cross entropy may, in some implementations, be used to quantify the difference between two or more distributions. Cross entropy loss may, in some implementations, be used to train the neural networks of the present disclosure. Cross entropy loss may, in some implementations, involve comparing a predicted probability to a ground truth probability. Other names of cross entropy loss include “logarithmic loss,” “logistic loss,” and “log loss”. A small cross entropy loss may indicate a better (e.g., more accurate) model. Cross entropy loss may be logarithmic. Cross entropy loss may, in some implementations, be applied to binary classification problems. In some implementations, a neural network may be equipped with a sigmoid activation unit at the output to generate a probability prediction. In the case of multi-class classifications, cross entropy may also be used. In such a case, a neural network trained to make multi-class predictions may, in some implementations, be equipped with one or more softmax activation functions at the output (e.g., where there is one output node for class that is to be predicted). Other loss calculation techniques which may be applied in the training of the neural networks of this disclosure include one or more of: Huber loss, Hinge loss, Categorical hinge loss, cosine similarity, Poisson loss, Logcosh loss, or mean squared logarithmic error loss (MSLE). Other loss calculation methods are described herein and may be applied to the training of any of the neural networks described in the present disclosure.

One or more of the neural networks of the present disclosure may, in some implementations, be trained, at least in part by a loss which is based on at least one of: a Point-wise Mesh Euclidean Distance (PMD) and an Earth Mover's Distance (EMD). Some implementations may incorporate a Hausdorff Distance (HD) calculation into the loss calculation. Computing the Hausdorff distance between two or more 3D representations (such as 3D meshes) may provide one or more technical improvements, in that the HD not only accounts for the distances between two meshes, but also accounts for the way that those meshes are oriented, and the relationship between the mesh shapes in those orientations (or positions or poses). Hausdorff distance may improve the comparison of two or more tooth meshes, such as two or more instances of a tooth mesh which are in different poses (e.g., such as the comparison of predicted setup to ground truth setup which may be performed in the course of computing a loss value for training a setups prediction neural network).

Reconstruction loss may compare a predicted output to a ground truth (or reference) output. Systems of this disclosure may compute reconstruction loss as a combination of L1 loss and MSE loss, as shown in the following line of pseudocode: reconstruction_loss=0.5*L1(all_points_target,all_points_predicted)+0.5*MSE(all_points_target,all_points_predicted). In the above example, all_points_target is a 3D representation (e.g., a 3D mesh or point cloud) corresponding to ground truth data (e.g., a ground truth tooth restoration design, or a ground truth example of some other 3D oral care representation). In the above example, all_points_predicted is a 3D representation (e.g., a 3D mesh or point cloud) corresponding to generated or predicted data (e.g., a generated tooth restoration design, or a generated example of some other kind of 3D oral care representation). Other implementations of reconstruction loss may additionally (or alternatively) involve L2 loss, mean absolute error (MAE) loss or Huber loss terms.

Reconstruction error may compare reconstructed output data (e.g., as generated by a reconstruction autoencoder, such as a tooth design which has been generated for use in generating a dental restoration appliance) to the original input data (e.g., the data which were provided to the input of the reconstruction autoencoder, such as a pre-restoration tooth). Systems of this disclosure may compute reconstruction error as a combination of L1 loss and MSE loss, as shown in the following line of pseudocode: reconstruction_error=0.5*L1(all_points_input, all_points_reconstructed)+0.5*MSE(all points_input, all_points_reconstructed). In the above example, all_points_input is a 3D representation (e.g., a 3D mesh or point cloud) corresponding to input data (e.g., the pre-restoration tooth design which was provided to a reconstruction autoencoder, or another 3D oral care representation which is provided to the input of an ML model). In the above example, all_points_reconstructed is a 3D representation (e.g., 3D mesh or point cloud) corresponding to reconstructed (or generated) data (e.g., a reconstructed tooth restoration design, or another example of a generated 3D oral care representation).

In other words, reconstruction loss is concerned with computing a difference between a predicted output and a reference output, whereas reconstruction error is concerned with computing a difference between a reconstructed output and an original input from which the reconstructed data are derived.

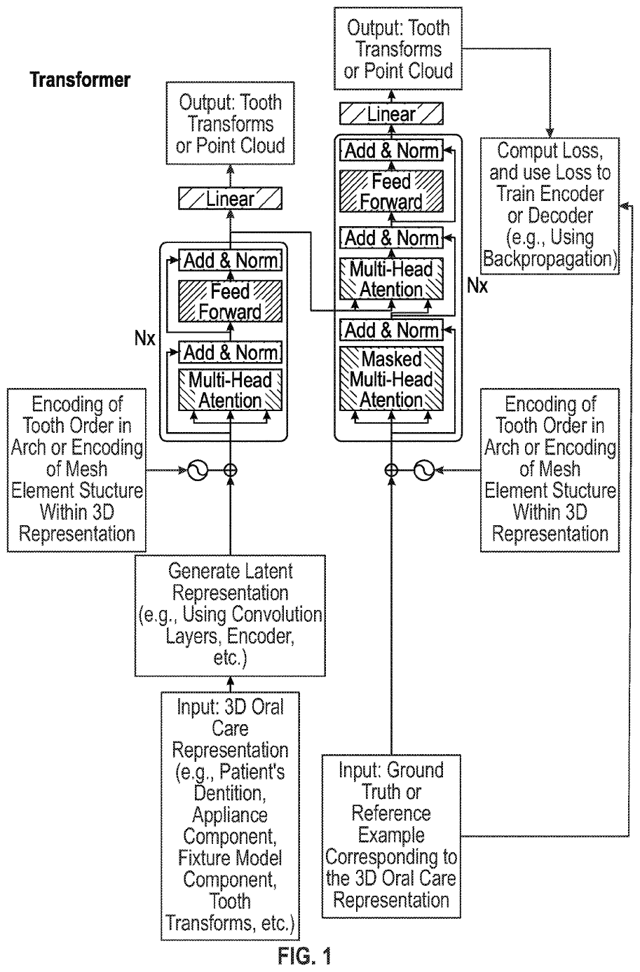

The entirety of the following paper is incorporated herein by reference in its entirety: “Attention Is All You Need”; Ashish Vaswani, Noam Shazeer, Niki Parmar, Niki Parmar, Llion Jones, Aidan N. Gomez, Lukasz Kaiser, Illia Polosukhin; NIPS 2017. The neural network-based models of this disclosure may provide additional advantages in implementations in which they are integrated with a neural network structure referred to as a “transformer.” FIG. 1 shows an example implementation of a transformer architecture.

Before recently developed models such as the transformer model, RNN-type models represented the state of the art for natural language processing (NLP). One example application of NLP is the generation of new text based upon prior words or text. Transformers have in turn provided significant improvements over GRU, LSTM and other such RNN-based NLP techniques due to an important attribute of the transformer model, which has the property of multi-headed attention. In some implementations, the NLP concept of multi-headed attention may describe the relationship between each word in a sentence (or paragraph or document or corpus of documents) and each other word in that sentence (or paragraph or document or corpus of documents). These relationships may be generated by a multiheaded attention module and may be encoded in vector form. This vector may describe how each word in a sentence (or paragraph or document or corpus of documents) should attend to each other word in that sentence (or paragraph or document or corpus of documents). RNN, LSTM and GRU models process a sequence, such a sentence, one word at a time from the start to the end of the sequence. Furthermore, the model may only account for a given subset (called a window) of the sentence when making a prediction. However, transformer-based models may, in some instances, account for the entirety of the preceding text by processing the sequence in its entirety in a single step. Transformer, RNN, LSTM, and GRU models can all be adapted for use in predictive models in digital dentistry and digital orthodontics, particularly for the setup prediction task. In some implementations, an exemplary transformer model for use with 3D meshes and 3D transforms in setups prediction (or other oral care techniques) may be adapted from the Bidirectional Encoder Representation from Transformers (BERT) and/or Generative Pre-Training (GPT) models. For example, a GPT (or BERT) model may first be trained on other data, such as text or documents data, and then be used in transfer learning. Such a transfer learning process may receive a previously trained GPT or BERT model, and then do further training using data comprising 3D oral care representations. Such transfer learning may be performed to train oral care models such as: segmentation, mesh cleanup, coordinate system prediction, setups prediction, validation of 3D oral care representations, transform prediction for placement of oral care meshes (e.g., teeth, hardware, appliance components, fixture model components), tooth restoration design generation (or generation of other 3D oral care representations—such as appliance components, fixture models or archforms), classification of 3D oral care representations, imputation of missing oral care parameters, clustering of clinicians or clustering of clinician preferences, or the like.

Oral care data may comprise one or more of (or combinations of): 3D representations of tooth (e.g., meshes, point clouds or voxels), sections of tooth meshes (such as subsets of mesh elements), tooth transforms (such as in matrix, vector and/or quaternion form, or combinations thereof), transforms for appliance components, transforms for fixture model components, and mesh coordinate system definitions (such as represented by transforms, for example, transformation matrices) and/or other 3D oral care representations described herein.

Transformers may be trained for generating transforms to position teeth into setups poses (or to place appliance components for use in appliance generation or to place fixture model components for use in fixture model generation). Some implementations may operate in an offline prediction context, and some implementations operation in an online reinforcement learning (RL) context. In some implementations, a transformer may be initially trained in an offline context and then undergo further fine-tuning training in the online context. In the offline prediction context, the transformer may be trained from a dataset of cohort patient case data. In the online RL context, the transformer may be trained from either a physics model, or a CAD model, for example. The transformer may learn from static data, such as transformations (e.g., trajectory transformer). In some implementations, the transform may provide a mapping from malocclusion to setup (e.g., receiving transformation matrices as input and generating transformation matrices as output). Some implementations of transformers may be trained to process 3D representations, such as 3D meshes, 3D point clouds or voxels (e.g., using a decision transformer) takes as input geometry (e.g., mesh, point cloud, voxels etc.), outputs transformations. The decision transformer may be coupled with a representation generation module that encodes representation of the patient's dentition (e.g., teeth), such as a VAE, a U-Net, an encoder, a transformer encoder, a pyramid encoder-decoder or a simple dense or fully connected network, or a combination thereof. In some implementations, the representation generation module (e.g., VAE, the U-Net, the encoder, the pyramid encoder-decoder or the dense network for generating the tooth representation) may be trained to generate the representation on one or more teeth. The representation generation module may be trained on all teeth in both arches, only the teeth within the same arch (either upper or lower), only anterior teeth, only posterior teeth, or some other subset of teeth. In some implementations, such a model may be trained on each individual tooth (e.g., an upper right cuspid), so that the model is trained or otherwise configured to generate highly accurate representations for an individual tooth. In some implementations, an encoder structure may encode such a representation. In some implementations, a decision transformer may learn in an online context, in an offline context or both. An online decision transformer may be trained (e.g., using RL techniques) to output action, state, and/or reward. In some implementations, transformations may be discretized, to allow for piecewise or stepwise actions.

In some implementations, a transformer may be trained to process an embedding of the arch (i.e., to predict transforms for multiple teeth concurrently), to predict a setup. In some implementations, embeddings of individual teeth may be concatenated into a sequence, and then input into the transformer. A VAE may be trained to perform this embedding operation, a U-Net may be trained to perform such an embedding, or a simple dense or fully connected network may be trained, or a combination thereof. In some implementations, the transformer-based techniques of this disclosure may predict an action for an individual tooth, or may predict actions for multiple teeth (e.g., predict transformations for each of multiple teeth).

A 3D mesh transformer may include a transformer encoder structure (which may encode oral care data) and may be followed by a transformer decoder structure. The 3D mesh transformer encoder may encode oral care data into a latent representation, which may be combined with attention information (e.g., to concatenate a vector of attention information to the latent representation). In some implementations, the attention information may help the decoder focus on the relevant oral care data during the decoding process (e.g., to focus on tooth order or mesh element connectivity), so that the transformer decoder can generate a useful output for the 3D mesh transformer (e.g., an output which may be used in the generation of an oral care appliance). Either or both of the transformer encoder or transformer decoder may generate a latent representation. The output of the transformer decoder (or transformer encoder) may be reconstructed using a decoder into, for example, one or more tooth transforms for a setup, one or more mesh element labels for segmentation, coordinate systems transforms for use in coordinate system generation, or one or more points of a point cloud or voxels or other mesh elements for another 3D representation). A transformer may include modules such as one or more of: multi-headed attention modules, feed forward modules, normalization modules, linear modules, and softmax modules, and convolution models for latent vector compression, and/or representation.

The encoder may be stacked one or more times, thereby further encoding the oral care data, and enabling different representations of the oral care data to be learned (e.g., different latent representations). These representations may be embedded with attention information (which may influence the decoder's focus to the relevant portions of the latent representation of the oral care data) and may be provided to the decoder in continuous form (e.g., as a concatenation of latent representations—such as latent vectors). In some implementations, the encoded output of the encoder (e.g., latent representations) may be used by downstream processing steps in the generation of oral care appliances. For example, the generated latent representation may be reconstructed into transforms (e.g., for the placement of teeth in setups, or the placement of appliance components or fixture model components), or may be reconstructed into 3D representations (e.g., 3D point clouds, 3D meshes or others disclosed herein). Stated another way, the latent representation which is generated by the transformer (e.g., containing continuously encoded attention information) may be provided to a decoder which has been configured to reconstruct the latent representation into the specific data structure which is required by a particular domain area. Continuously encoded attention information may include attention information which has undergone processing by multiple multi-headed attention modules within the transformer encoder or transformer decoder, to name one example. Furthermore, a loss may be computed for a particular domain using data from that domain. The loss calculation may train the transformer decoder to accurately reconstruct the latent representation into the output data structure pertaining to a particular domain.

For example, when the decoder generates a transform for an orthodontic setup, the decoder may be configured with outputs that describe, for example, the 16 real values which comprise a 4×4 transformation matrix (other data structures for describing transforms are possible). Stated a different way, the latent output generated by the transformer encoder (or transformer decoder) may be used to predict setups tooth transforms for one or more teeth, to place those teeth in setup positions (e.g., either final setups or intermediate stages). Such a transformer encoder (or transformer decoder) may be trained, at least in part using a reconstruction loss (or a representation loss, among others described herein) function, which may compare predicted transforms to ground truth (or reference) transforms.

In a further example, when the decoder generates a transform for a tooth coordinate system, the decoder may be configured with outputs that describe, for example, the 16 real values which comprise a 4×4 transformation matrix (other data structures for describing transforms are possible). Stated a different way, the latent output generated by the transformer encoder (or transformer decoder) may be used to predict local coordinate systems for one or more teeth. Such a transformer encoder (or transformer decoder) may be trained, at least in part using a representation loss (or a reconstruction loss, among others described herein) function, which may compare predicted coordinate systems to ground truth (or reference) coordinate systems.

In a further example, when the decoder generates a 3D point cloud (or other 3D representation—such as 3D mesh, voxelized representation, or the like), the decoder may be configured with outputs that describe, for example, one or more 3D points (e.g., comprising XYZ coordinates). Stated a different way, the latent output generated by the transformer encoder (or transformer decoder) may be used to predict mesh elements for a generated (or modified) 3D representation. Such a transformer encoder (or transformer decoder) may be trained, at least in part using a reconstruction loss (or an L1, L2 or MSE loss, among others described herein) function, which may compare predicted 3D representations to ground truth (or reference) 3D representations.

In a further example, when the decoder generates mesh element labels for 3D representation segmentation or 3D representation cleanup, the decoder may be configured with outputs that describe, for example, labels for one or more mesh elements. Stated a different way, the latent output generated by the transformer encoder (or transformer decoder) may be used to predict mesh element labels for mesh segmentation or mesh cleanup. Such a transformer encoder (or transformer decoder) may be trained, at least in part using a cross entropy loss (or others described herein) function, which may compare predicted mesh element labels to ground truth (or reference) mesh element labels.

Multi-headed attention and transformers may be advantageously applied to the setups-generation problem. Multi-headed attention is a module in a 3D transformer encoder network which computes the attention weights for the provided oral care data and produces an output vector with encoded information on how each example of oral care data should attend to each other oral care data in an arch. An attention weight is a quantification of the relationship between pairs of oral care data.

A 3D representation of oral care data (e.g., comprising voxels, a point cloud, or a 3D mesh composed of vertices, faces or edges) may be provided to the transformer. The 3D representation may describe the patient's dentition, a fixture model (or components of a fixture model), an appliance (or components of an appliance), or the like. In some implementations, a transformer decoder (or a transformer encoder) may be equipped with multi-head attention. Multi-headed attention may enable the transformer decoder (or transformer encoder) to attend to different portions of the 3D representation of oral care data. For example, multi-headed attention may enable the transformer to attend to mesh elements within local neighborhoods (or cliques), or to attend to global dependencies between mesh elements (or cliques). For example, multi-headed attention may enable a transformer for setups prediction (e.g., a setups prediction model which is based on a transformer) to generate a transform for a tooth, and to substantially concurrently attend to each of the other teeth in the arch while that transform is generated. Stated another way, the transform for each tooth may be generated in light of the poses of one or more other teeth in the arch, leading to a more accurate transform (e.g., a transform which conforms more closely to the ground truth or reference transform). In the example of 3D representation generation (e.g., the generation of a 3D point cloud), a transformer model may be trained to generate a tooth restoration design. Multi-headed attention may enable the transformer to attend to multiple portions of the tooth (or to the surfaces of the adjacent teeth) while the tooth undergoes the generative process. For example, the transformer for restoration design generation may generate the mesh elements for the incisal edge of an incisor while, at least substantially concurrently, attending to the mesh elements of the mesial, distal, facial or lingual surfaces of the incisor. The result may be the generation of mesh elements to form an incisal edge for the tooth which merges seamlessly with the adjacent surfaces of the tooth. This use of multi-headed attentions results in more accurate modeling of the distribution of the training dataset, over techniques which do not apply multi-headed attention.

In some implementations of the present disclosure, one or more attention vectors may be generated which describe how aspects of the oral care data interacts with other aspects of the oral care data associated with the arch. In some implementations, the one or more attention vectors may be generated to describe how one or more portions of a tooth T1 interact with one or more portions of a tooth T2, a tooth T3, a tooth T4, and so one. A portion of a mesh may be described as a set of mesh elements, as defined herein. In some implementations, the interacting portions of tooth T1 and tooth T2 may be determined, in part, through the calculation of mesh correspondences, as described herein. Any of these models (RNN, GRU, LSTM and Transformer) may be advantageously applied to the task of setups transform prediction, such as in the models described herein. A transformer may be particularly advantageous in that a transformer may enable the transforms for multiple teeth, or even an entire arch to be generated at once, rather than individually, as may be the case with some other models, such as an encoder structure. In other implementations, attention-free transformers may be used to make predictions based on oral care data.

One implementation of the GDL Setups neural network model may include a representation generation module (e.g., containing a U-Net structure, an autoencoder encoder, a transformer encoder, another type of encoder-decoder structure, or an encoder, etc.) which may provide its output to a module which is trained to generate tooth transformers (e.g., a set of fully connected layers with optional skip connections, or an encoder structure) to generate the prediction of a transform for each individual tooth. Skip connections may, in some implementations, connect the outputs of a particular layer in a neural network to the inputs of another later in the neural network (e.g., a layer which is not immediately adjacent to the originating layer). The transform-generation module (e.g., an encoder) may handle the transform prediction one tooth at a time. Other implementations may replace this encoder structure with a transformer (e.g., transformer encoder or transformer decoder), which may handle all the predictions for all teeth substantially concurrently.

Stated another way, a transformer may be configured to receive a large number of input values, larger than some other neural network models (e.g., than a typical MLP). This is because an increased number of inputs may be accommodated by the transformer, the predictions corresponding to those inputs may be generated substantially concurrently. The representation generation module (e.g., U-Net structure) may provide its output to the transformer, and the transformer may generate the setups transforms for all of the several teeth at once, with the technical advantage of improved accuracy (because the transforms for each tooth is generated in light of the transform for each of the adjacent or nearby teeth—leading to fewer collisions and better conformance with the goals of treatment). A transformer may be trained to output a transformation, such as a transform encoded by a 4×4 matrix (or some other size), a quaternion, a translation vector, Euler angles or some other form. The transformation may place a tooth into a setups pose, may place a fixture model component into a pose suitable for fixture model generation, or may place an appliance component into a pose suitable for appliance generation (e.g., dental restoration appliance, clear tray aligner, etc.). In some implementations, the transform may define a coordinate system for aspects of the patient's dentition, such as a tooth mesh (e.g., a local coordinate system for a tooth). In some implementations, the inputs to the transformer may first be encoded using a neural network (e.g., a latent representation or embedding may be generated), such as one or more linear layers, and/or one or more convolutional layers. In some implementations, the transformer may first be trained on an offline dataset, and subsequently be trained using a secondary actor-critic network, which may enable online reinforcement learning.

Transformers may, in some implementations, enable large model capacity and/or enable an attention mechanism (e.g., the capability to pay attention and respond to certain inputs). The attention mechanisms (e.g., multi-headed attention) that are found within transformers may enable intra-sequence relationships to be encoded into neural network features. Intra-sequence relationships may be encoded, for example, by associating an order number (e.g., 1, 2, 3, etc.) with each tooth in an arch, or by associating an order number with each mesh element in a 3D representation (e.g., of a tooth). In implementations where latent vectors of teeth are provided to the transformer, intra-sequence relationships may be encoded, for example, by associating an order number (e.g., 1, 2, 3, etc.) with each element in the latent vector.

Transformers may be scaled by increasing the number of attention heads and/or by increasing the number of transformer layers. Stated differently, one or more aspects of a transformer may be independently trained to handle discrete tasks, and later combined to allow the resulting transformer to perform all of the tasks for which the individual components had been trained, without degrading the predictive accuracy of the neural network. Scaling a convolutional network may be more difficult because the models may be less malleable or may be less interchangeable.

Performing convolutions as described herein may result in systems and techniques that are rotation and translation invariant, which leads to improved generalization, because a convolution model may not need to account for the manner in which the input data in rotated or translated. Transformers configured as described herein may be permutation invariant, because intra-sequence relationships may be encoded into neural network features.

In some implementations for the generation or modification of 3D oral care representations, transformers may be combined with convolution-based neural networks, such as by vertically stacking convolution layers and attention layers. Stacking transformer blocks with convolutional blocks enables the resulting structure to have the translation invariance of convolution, and also the permutation invariance of a transformer. Such stacking may improve model capacity and/or model generalization. CoAtNet is an example of a network architecture which combines convolutional and attention-based elements and may be applied to the processing of oral care data. In some instances, a network for the modification or generation of 3D oral care representations may be trained, at least in part, from CoAtNet (or another model that combines convolution and self-attention/transformers) using transfer learning.

The techniques of this disclosure may include operations such as 3D convolution, 3D pooling, 3D unconvolution and 3D unpooling. 3D convolution may aid segmentation processing, for example in down sampling a 3D mesh. 3D un-convolution undoes 3D convolution, for example, in a U-Net. 3D pooling may aid the segmentation processing, for example in summarized neural network feature maps. 3D un-pooling undoes 3D pooling, for example in a U-Net. These operations may be implemented by way of one or more layers in the predictive or generative neural networks described herein. These operations may be applied directly on mesh elements, such as mesh edges or mesh faces. These operations provide for technical improvements over other approaches because the operations are invariant to mesh rotation, scale, and translation changes. In general, these operations depend on edge (or face) connectivity, therefore these operations remain invariant to mesh changes in 3D space as long as edge (or face) connectivity is preserved. That is, the operations may be applied to an oral care mesh and produce the same output regardless of the orientation, position, or scale of that oral care mesh, which may lead to data precision improvement. MeshCNN is a general-purpose deep neural network library for 3D triangular meshes, which can be used for tasks such as 3D shape classification or mesh element labelling (e.g., for segmentation or mesh cleanup). MeshCNN implements these operations on mesh edges. Other toolkits and implementations may operate on edges or faces.

In some implementations of the techniques of this disclosure, neural networks may be trained to operate on 2D representations (such as images). In some implementations of the techniques of this disclosure, neural networks may be trained to operate on 3D representations (such as meshes or point clouds). An intraoral scanner may capture 2D images of the patient's dentition from various views. An intraoral scanner may also (or alternatively) capture 3D mesh or 3D point cloud data which describes the patient's dentition. According to various techniques, autoencoders (or other neural networks described herein) may be trained to operate on either or both of 2D representations and 3D representations.

A 2D autoencoder (comprising a 2D encoder and a 2D decoder) may be trained on 2D image data to encode an input 2D image into a latent form (such as a latent vector or a latent capsule) using the 2D encoder, and then reconstruct a facsimile of the input 2D image using the 2D decoder. In the case of a handheld mobile app which has been developed for such analysis (e.g., for the analysis of dental anatomy), 2D images may be readily captured using one or more of the onboard cameras. In other examples, 2D images may be captured using an intraoral scanner which is configured for such a function. Among the operations which may be used in the implementation a 2D autoencoder (or other 2D neural network) for 2D image analysis are 2D convolution, 2D pooling and 2D reconstruction error calculation.

2D image convolution may involve the “sliding” of a kernel across a 2D image and the calculation of elementwise multiplications and the summing of those elementwise multiplications into an output pixel. The output pixel that results from each new position of the kernel is saved into an output 2D feature matrix. In some implementations, neighboring elements (e.g., pixels) may be in well-defined locations (e.g., above, below, left and right) in a rectilinear grid.

### 2D Pooling:

A 2D pooling layer may be used to down sample a feature map and summarize the presence of certain features in that feature map.

2D reconstruction error may be computed between the pixels of the input and reconstructed images. The mapping between pixels may be well understood (e.g., the upper pixel [23,134] of the input image is directly compared to pixel [23,134] of the reconstructed image, assuming both images have the same dimensions).

Among the advantages provided by the 2D autoencoder-based techniques of this disclosure is the ease of capturing 2D image data with a handheld device. In some instances, where outside data sources provide the data for analysis, there may be instances where only 2D image data are available. When only 2D image data are available, then analysis using a 2D autoencoder is warranted.

Modern mobile devices (such as commercially available smartphones) may also have the capability of generating 3D data (e.g., using multiple cameras and stereophotogrammetry, or one camera which is moved around the subject to capture multiple images from different views, or both), which in some implementations, may be arranged into 3D representations such as 3D meshes, 3D point clouds and/or 3D voxelized representations. The analysis of a 3D representation of the subject may in some instances provide technical improvements over 2D analysis of the same subject. For example, a 3D representation may describe the geometry and/or structure of the subject with less ambiguity than a 2D representation (which may contain shadows and other artifacts which complicate the depiction of depth from the subject and texture of the subject). In some implementations, 3D processing may enable technical improvements because of the inverse optics problem which may, in some instances, affect 2D representations. The inverse optics problem refers to the phenomenon where, in some instances, the size of a subject, the orientation of the subject and the distance between the subject and the imaging device may be conflated in a 2D image of that subject. Any given projection of the subject on the imaging sensor could map to an infinite count of {size, orientation, distance}pairings. 3D representations enable the technical improvement in that 3D representations remove the ambiguities introduced by the inverse optics problem.

A device that is configured with the dedicated purpose of 3D scanning, such as a 3D intraoral scanner (or a CT scanner or MRI scanner), may generate 3D representations of the subject (e.g., the patient's dentition) which have significantly higher fidelity and precision than is possible with a handheld device. When such high-fidelity 3D data are available (e.g., in the application of oral care mesh classification or other 3D techniques described herein), the use of a 3D autoencoder is offers technical improvements (such as increased data precision), to extract the best possible signal out of those 3D data (i.e., to get the signal out of the 3D crown meshes used in tooth classification or setups classification).

A 3D autoencoder (comprising a 3D encoder and a 3D decoder) may be trained on 3D data representations to encode an input 3D representation into a latent form (such as a latent vector or a latent capsule) using the 3D encoder, and then reconstruct a facsimile of the input 3D representation using the 3D decoder. Among the operations which may be used to implement a 3D autoencoder for the analysis of a 3D representation (e.g., 3D mesh or 3D point cloud) are 3D convolution, 3D pooling and 3D reconstruction error calculation.

For each mesh element, a 3D convolution may be performed to aggregate local features from nearby mesh elements. Processing may be performed above and beyond the techniques for 2D convolution, to account for the differing count and locations of neighboring mesh elements (relative to a particular mesh element). A particular 3D mesh element may have a variable count of neighbors and those neighbors may not be found in expected locations (as opposed to a pixel in 2D convolution which may have a fixed count of neighboring pixels which may be found in known or expected locations). In some instances, the order of neighboring mesh elements may be relevant to 3D convolution.

A 3D pooling operation may enable the combining of features from a 3D mesh (or other 3D representation) at multiple scales. 3D pooling may iteratively reduce a 3D mesh into mesh elements which are most highly relevant to a given application (e.g., for which a neural network has been trained). Similarly to 3D convolution, 3D pooling may benefit from special processing beyond that entailed in 2D convolution, to account for the differing count and locations of neighboring mesh elements (relative to a particular mesh element). In some instances, the order of neighboring mesh elements may be less relevant to 3D pooling than to 3D convolution.

3D reconstruction error may be computed using one or more of the techniques described herein, such as computing Euclidean distances between corresponding mesh elements, between the two meshes. Other techniques are possible in accordance with aspects of this disclosure. 3D reconstruction error may generally be computed on 3D mesh elements, rather than the 2D pixels of 2D reconstruction error. 3D reconstruction error may enable technical improvements over 2D reconstruction error, because a 3D representation may, in some instances, have less ambiguity than a 2D representation (i.e., have less ambiguity in form, shape and/or structure). Additional processing may, in some implementations, be entailed for 3D reconstruction which is above and beyond that of 2D reconstruction, because of the complexity of mapping between the input and reconstructed mesh elements (i.e., the input and reconstructed meshes may have different mesh element counts, and there may be a less clear mapping between mesh elements than there is for the mapping between pixels in 2D reconstruction). The technical improvements of 3D reconstruction error calculation include data precision improvement.

A 3D representation may be produced using a 3D scanner, such as an intraoral scanner, a computerized tomography (CT) scanner, ultrasound scanner, a magnetic resonance imaging (MRI) machine or a mobile device which is enabled to perform stereophotogrammetry. A 3D representation may describe the shape and/or structure of a subject. A 3D representation may include one or more 3D mesh, 3D point cloud, and/or a 3D voxelized representation, among others. A 3D mesh includes edges, vertices, or faces. Though interrelated in some instances, these three types of data are distinct. The vertices are the points in 3D space that define the boundaries of the mesh. These points would alternatively be described as a point cloud but for the additional information about how the points are connected to each other, as described by the edges. An edge is described by two points and can also be referred to as a line segment. A face is described by a number of edges and vertices. For instance, in the case of a triangle mesh, a face comprises three vertices, where the vertices are interconnected to form three contiguous edges. Some meshes may contain degenerate elements, such as non-manifold mesh elements, which may be removed, to the benefit of later processing. Other mesh e-processing operations are possible in accordance with aspects of this disclosure. 3D meshes are commonly formed using triangles, but may in other implementations be formed using quadrilaterals, pentagons, or some other n-sided polygon. In some implementations, a 3D mesh may be converted to one or more voxelized geometries (i.e., comprising voxels), such as in the case that sparse processing is performed. The techniques of this disclosure which operate on 3D meshes may receive as input one or more tooth meshes (e.g., arranged in one or more dental arches). Each of these meshes may undergo pre-processing before being input to the predictive architecture (e.g., including at least one of an encoder, decoder, pyramid encoder-decoder and U-Net). This pre-processing may include the conversion of the mesh into lists of mesh elements, such as vertices, edges, faces or in the case of sparse processing—voxels. For the chosen mesh element type or types, (e.g., vertices), feature vectors may be generated. In some examples, one feature vector is generated per vertex of the mesh. Each feature vector may contain a combination of spatial and/or structural features, as specified in the following table:

TABLE 1

| Element | Spatial Features | Structural Features |
|---|---|---|
| Edges | XYZ position of an edge | Edge curvature (depends on a |
| | midpoint, XYZ positions | connectivity neighborhood, |
| | of the edge vertices, | average curvature of two |
| | or the normal vector at | vertices), dihedral angles, edge |
| | an edge midpoint | length, density measure such as |
| | (average of the normal | a count of incident edges (i.e., a |
| | vectors of two vertices). | count of the other neighboring |
| | | edges which share the vertices |
| | | of that edge). |
| Faces | XYZ position of a face | Face curvature (average |
| | centroid, surface | curvature of the vertices of the |
| | normal vector. | face), face area, density measure |
| | | such as count of adjacent faces |
| | | (i.e., which share at least one |
| | | edge with the face). |
| Points | XYZ position | Density measure such as the |
| | | count of neighboring points |
| | | within a radius of the point |
| Vertices | XYZ position, normal | Vertex curvature, density |
| | vector (weighted | measure such as the count of |
| | average of the normal | vertices within a radius of the |
| | vectors of the | vertex, density measure such as |
| | connecting faces | the count of incident edges. |
| | for the vertex). | |
| Voxels | XYZ centroid. | Volume, [height × depth × |
| | | width] dimensions, density measure |
| | | such as a count of contained |
| | | vertices, density measure such as |
| | | count of intersected faces, |
| | | density measure such as count of |
| | | intersected edges. |

Table 1 discloses non-limiting examples of mesh element features. In some implementations, color (or other visual cues/identifiers) may be considered as a mesh element feature in addition to the spatial or structural mesh element features described in Table 1. As used herein (e.g., in Table 1), a point differs from a vertex in that a point is part of a 3D point cloud, whereas a vertex is part of a 3D mesh and may have incident faces or edges. A dihedral angle (which may be expressed in either radians or degrees) may be computed as the angle (e.g., a signed angle) between two connected faces (e.g., two faces which are connected along an edge). A sign on a dihedral angle may reveal information about the convexity or concavity of a mesh surface. For example, a positively signed angle may, in some implementations, indicate a convex surface. Furthermore, a negatively signed angle may, in some implementations, indicate a concave surface. To calculate the principal curvature of a mesh vertex, directional curvatures may first be calculated to each adjacent vertex around the vertex. These directional curvatures may be sorted in circular order (e.g., 0, 49, 127, 210, 305 degrees) in proximity to the vertex normal vector and may comprise a subsampled version of the complete curvature tensor. Circular order means: sorted in by angle around an axis. The sorted directional curvatures may contribute to a linear system of equations amenable to a closed form solution which may estimate the two principal curvatures and directions, which may characterize the complete curvature tensor. Consistent with Table 1, a voxel may also have features which are computed as the aggregates of the other mesh elements (e.g., vertices, edges and faces) which either intersect the voxel or, in some implementations, are predominantly or fully contained within the voxel. Rotating the mesh may not change structural features but may change spatial features. And, as described elsewhere in this disclosure, the term “mesh” should be considered in a non-limiting sense to be inclusive of 3D mesh, 3D point cloud and 3D voxelized representation. In some implementations, apart from mesh element features, there are alternative methods of describing the geometry of a mesh, such as 3D keypoints and 3D descriptors. Examples of such 3D keypoints and 3D descriptors are found in “TONIONI A, et al. in ‘Learning to detect good 3D keypoints.’, Int J Comput. Vis. 2018 Vol 0.126, pages 1-20.”. 3D keypoints and 3D descriptors may, in some implementations, describe extrema (either minima or maxima) of the surface of a 3D representation. In some implementations, one or more mesh element features may be computed, at least in part, via deep feature synthesis (DFS), e.g. as described in: J. M. Kanter and K. Veeramachaneni, “Deep feature synthesis: Towards automating data science endeavors,” 2015 IEEE International Conference on Data Science and Advanced Analytics (DSAA), 2015, pp. 1-10, doi: 10.1109/DSAA.2015.7344858.

Representation generation neural networks based on autoencoders, U-Nets, transformers, other types of encoder-decoder structures, convolution and/or pooling layers, or other models may benefit from the use of mesh element features. Mesh element features may convey aspects of a 3D representation's surface shape and/or structure to the neural network models of this disclosure. Each mesh element feature describes distinct information about the 3D representation that may not be redundantly present in other input data that are provided to the neural network. For example, a vertex curvature may quantify aspects of the concavity or convexity of the surface of a 3D representation which would not otherwise be understood by the network. Stated differently, mesh element features may provide a processed version of the structure and/or shape of the 3D representation; data that would not otherwise be available to the neural network. This processed information is often more accessible, or more amenable for encoding by the neural network. A system implementing the techniques disclosed herein has been utilized to run a number of experiments on 3D representations of teeth. For example, mesh element features have been provided to a representation generation neural network which is based on a U-Net model, and also to a representation generation model based on a variational autoencoder with continuous normalizing flows. Based on experiments, it was found that systems using a full complement of mesh element features (e.g., “XYZ” coordinates tuple, “Normal vector”, “Vertex Curvature”, Points-Pivoted, and Normals-Pivoted) were at least 3% more accurate than systems that did not. Points-Pivoted describes “XYZ” coordinates tuples that have local coordinate systems (e.g., at the centroid of the respective tooth). Normals-Pivoted describes “Normal Vectors” which have local coordinate systems (e.g., at the centroid of the respective tooth). Furthermore, training converges more quickly when the full complement of mesh element features are used. Stated another way, the machine learning models trained using the full complement of mesh element features tended to be more accurate more quickly (at earlier epochs) than systems which did not. For an existing system observed to have a historical accuracy rate of 91%, an improvement in accuracy of 3% reduces the actual error rate by more than 30%.

Predictive models which may operate on feature vectors of the aforementioned features include but are not limited to: GDL Setups, RL Setups, VAE Setups, Capsule Setups, MLP Setups, Diffusion Setups, PT Setups, Similarity Setups, Tooth Classification, Setups Classification, Setups Comparison, VAE Mesh Element Labeling, MAE Mesh In-filling, Mesh Reconstruction Autoencoder, Validation Using Autoencoders, Mesh Segmentation, Coordinate System Prediction, Mesh Cleanup, Restoration Design Generation, Appliance Component Generation and/or Placement, and Archform Prediction. Such feature vectors may be presented to the input of a predictive model. In some implementations, such feature vectors may be presented to one or more internal layers of a neural network which is part of one or more of those predictive models.

As described herein, tooth movements specify one or more tooth transformations that can be encoded in various ways to specify tooth positions and orientations within the setup and are applied to 3D representations of teeth. For instance, according to particular implementations, the tooth positions can be cartesian coordinates of a tooth's canonical origin location which is defined in some semantic context. Tooth orientations can be represented as rotation matrices, unit quaternions, or other 3D rotation representations such as Euler angles with respect to a frame of reference (either global or local). Dimensions are real valued 3D spatial extents and gaps can be binary presence indicators or real valued gap sizes between teeth especially in instances when certain teeth are missing. In some implementations, tooth rotations may be described by 3×3 matrices (or by matrices of other dimensions). Tooth position and rotation information may, in some implementations, be combined into the same transform matrix, for example, as a 4×4 matrix, which may reflect homogenous coordinates. In some instances, affine spatial transformation matrices may be used to describe tooth transformations, for example, the transformations which describe the maloccluded pose of a tooth, an intermediate pose of a tooth and/or a final setup pose of a tooth. Some implementations may use relative coordinates, where setup transformations are predicted relative to malocclusion coordinate systems (e.g., a malocclusion-to-setup transformation is predicted instead of a setup coordinate system directly). Other implementations may use absolute coordinates, where setup coordinate systems are predicted directly for each tooth. In the relative mode, transforms can be computed with respect to the centroid of each tooth mesh (vs the global origin), which is termed “relative local.” Some of the advantages of using relative local coordinates include eliminating the need for malocclusion coordinate systems (landmarking data) which may not be available for all patient case datasets. Some of the advantages of using absolute coordinates include simplifying the data preprocessing as mesh data are originally represented as relative to the global origin. These details about tooth position encoding and tooth orientation encoding may, in some implementations, also apply one or more of the neural networks models of the present disclosure, including but not limited to: GDL Setups, RL Setups, VAE Setups, Capsule Setups, MLP Setups, Diffusion Setups, PT Setups, Similarity Setups, FDG Setups, Setups Classification, Setups Comparison, VAE Mesh Element Labeling, MAE Mesh In-filling, Mesh Reconstruction VAE, and Validation Using Autoencoders.

According to particular implementations, convolution layers in the various 3D neural networks described herein may use edge data to perform mesh convolution. The use of edge information guarantees that the model is not sensitive to different input orders of 3D elements. In addition to or separate from using edge data, the convolution layers may use vertex data to perform mesh convolution. The use of vertex information is advantageous in that there are typically fewer vertices than edges or faces, so vertex-oriented processing may lead to a lower processing overhead and lower computational cost. In addition to or separate from using edge data or vertex data, the convolution layers may use face data to perform mesh convolution. Furthermore, in addition to or separate from using edge data, vertex data, or face data, the convolution layers may use voxel data to perform mesh convolution. The use of voxel information is advantageous in that, depending on the granularity chosen, there may be significantly fewer voxels to process compared to the vertices, edges or faces in the mesh. Sparse processing (with voxels) may lead to a lower processing overhead and lower computational cost (especially in terms of computer memory or RAM usage).

Examples of oral care metrics include Orthodontic Metrics (OM) and Restoration Design Metrics (RDM). RDM may describe the shape and/or form of one or more 3D representations of teeth for use in dental restoration. One use case example is in the creation of one or more dental restoration appliances. Another use case example is in the creation of one or more veneers (such as a zirconia veneer). Some RDM may quantify the shape and/or other characteristics of a tooth. Other RDM may quantify relationships (e.g., spatial relationships) between two or more teeth. RDM differ from restoration design parameters (RDP) in that restoration design metrics define a current state of a patient's dentition, whereas restoration design parameters serve as specifications to a machine learning or other optimization model to generate desired tooth shapes and/or forms. RDM describe the shapes of the teeth currently (e.g., in a starting or mal condition). Restoration design parameters specify how an oral care provider (such as a dentist or dental technician) intends for the teeth to look after the completion of restoration treatment. Either or both of RDM and RDP may be provided a neural network or other machine learning or optimization algorithm for the purpose of dental restoration. In some implementations, RDM may be computed on the pre-restoration dentition of the patient (i.e., the primary implementation). In other implementations, RDM may be computed on the post-restoration dentition of the patient. A restoration design may comprise one or more teeth and may be referred to as a restoration arch. Restoration design generation may involve the generation of an improved geometry and/or structure of one or more teeth in a restoration arch.

Aspects of RDM calculation are described below. In some implementations, RDM may be measured, for example, through locating landmarks in the teeth (or gums, hardware and/or other elements of the patient's dentition), and the measurements of distances between those landmarks, or otherwise made in relation to those landmarks. In some implementations, one or more neural networks or other machine learning models may be trained to identify or extract one or more RDM from one or more 3D representations of teeth (or gums, hardware and/or other elements of the patient's dentition). Techniques of this disclosure may use RDM in various ways. For instance, in some implementations, one or more neural networks or other machine learning models may be trained to classify or label one or more setups, arches, dentitions or other sets of teeth based at least in part on RDM. As such, in these examples, RDMs form a part of the training data used for training these models.

Aspects of a tooth mesh reconstruction autoencoder may be used in accordance with techniques of this disclosure are described below. An autoencoder for restoration design generation is disclosed in U.S. Provisional Application No. 63/366,514. This autoencoder (e.g., a variational autoencoder or VAE) takes as input a tooth mesh (or other 3D representation) that reflects a mal state (i.e., the pre-restoration tooth shape). The encoder component of the autoencoder encodes that tooth mesh to a latent form (e.g., a latent vector). Modifications may be applied to this latent vector (e.g., based on a mapping of the latent space through prior experiments), for the purpose of altering the geometry and/or structure of the eventual reconstructed mesh. Additional vectors may, in some implementations, be included with the latent vector (e.g., through concatenation), and the resulting concatenation of vectors may be reconstructed by way of the decoder component of the autoencoder into a reconstructed tooth mesh which is a facsimile of the input tooth mesh.

RDM and RDP may also be used as neural network inputs in the execution phase, in accordance with aspects of this disclosure. In some implementations, one or more RDM may be concatenated with the input to the encoder, for the purpose of telling the encoder specific information about the input 3D tooth representation. In some implementations, one or more RDM may be concatenated with the latent vector, before reconstruction, for the purpose of providing the decoder component with specific information about the input 3D tooth representation. Furthermore, in some implementations, one or more restoration design parameters (RDP) may be concatenated with the input to the encoder component, for the purpose of providing the encoder specific information about the input 3D tooth representation. Likewise, in some implementations, one or more restoration design parameters (RDP) may be concatenated with the latent vector, before reconstruction, for the purpose of providing the decoder specific information about the input 3D tooth representation.

In this way, either or both of RDM and RDP may be introduced to the functioning of an autoencoder (e.g., a tooth reconstruction autoencoder), and serve to influence the geometry and/or structure of the reconstructed restoration design (i.e., influence the shape of the tooth on the output of the autoencoder). In some implementations, the variational autoencoder of US Provisional Application No. U.S. 63/366,514 may be replaced by a capsule autoencoder (e.g., instead of encoding the tooth mesh to a latent vector, the tooth mesh is encoded into one or more latent capsules).

In some implementations, clustering or other unsupervised techniques may be performed on RDM to cluster one or more setups, arches, dentitions or other sets of teeth based on the restoration characteristics of the teeth. Such clusters may be useful in treatment planning, as the clusters provide insight into categories of patients with different treatment needs. This information may be instructive to clinicians as they learn about possible treatment options. In some instances, best practices may be identified (such as default RDP values) for patient cases that fall into one or another cluster (e.g., as determined by a similarity measure, as in k-NN). After a new case is classified into a particular cluster, information about the relevant best practices may be provided to the clinician who is responsible for processing the case. Such default values may, in some instances, undergo further tuning or modifications.

Case Assignment: Such clusters may be used to gain further insight into the kinds of patient cases which exist in a dataset. Analysis of such clusters may reveal that patient treatment cases with certain RDM values (or ranges of values) may take less time to treat (or alternatively more time to treat). Cases which take more time to treat (or are otherwise more difficult) may be assigned to experienced or senior technicians for processing. Cases which take less time to treat may be assigned to newer or less-experienced techniques for processing. Such an assignment may be further aided by finding correlations between RDM values for certain cases and the known processing durations associated with those cases.

The following RDM may be measured and used in the creation of either or both of dental restoration appliances and veneers {veneers are a type of dental restoration appliance}, with the objective of making the resulting teeth natural looking. Symmetry is generally a preferred facet. There may be differences between patients based on demographic differences. The generation of dental restoration appliances may benefit from some or all of the following RDM. Shade and translucency may pertain, in particular, to the creation of veneers, though some implementations of dental restoration appliances may also consider this information.

Examples of inter-tooth RDM are described as follows.

1) Bilateral Symmetry and/or Ratios: A measure of the symmetry between one or more teeth and one or more other teeth on opposite sides of the dental. For example, for a pair of corresponding teeth, a measure of the width of each tooth. In one instance, the one tooth is of normal width, and the other tooth is too narrow. In another instance, both teeth are of normal width. The following is a list of attributes that can be measured for a tooth, and compared to the corresponding measurement for one or more corresponding teeth: a) width—mesial to distal distance; b) length—gingival to incisal distance; c) diagonal—distance across the tooth, e.g., from the mesial gingival corner to the distal incisal corner (this measure is one of many that can be used to quantify the shape of teeth beyond length and width). Ratios between a and b may be computed, such as a/b or b/a. Such ratios can be indicative of whether spatial symmetry exists (e.g., by measuring the ratio ab on the left side and measuring the ratio ab on the right side, then compare the left and right ratios). In some implementations, where spatial symmetry is “off”, the length, width and/or ratios may not match. Such a ratio may, in some implementations, be computed relative to a standard. A number of esthetic standards are available in the dental literature. Examples include Golden Proportion and Recurring Esthetic Dental Proportion. In some implementations, spatial symmetry may be measured on a pair of teeth, where one tooth is on the right side of the arch, and the other tooth is on the left side of the arch.

2) Proportions of Adjacent Teeth: Measure the width proportions of adjacent teeth as measured as a projection along an arch onto a plane (e.g., a plane that is situated in front of the patient's face). The ideal proportions for use in the final restoration design can be, for example, the so-called golden proportions. The golden proportions relate adjacent teeth, such as central incisors and lateral incisors. This metric pertains to the measuring of these proportions as the proportions exist in the pre-restoration mal dentition. The ideal golden proportions are 1.6, 1, 0.6, for the central incisor, lateral incisor and cuspid, on a particular side (either left or right) for a particular arch (e.g., the upper arch). If one or more of these proportion values is off (e.g., in the case of “peg laterals”), the patient may wish for dental restoration treatment to correct the proportions.

3) Arch Discrepancies: A measure of any size discrepancies between the upper arch and lower arch, for example, pertaining to the widths of the teeth, for the purpose of dental restoration. For example, techniques of this disclosure may make adjacent tooth width proportion measurements in the upper arch and in the lower arch. In some implementations, Bolton analysis measurements may be made by measuring upper widths, lower widths, and proportions between those quantities. Arch discrepancies may be described in absolute measurements (e.g., in mm or other suitable units) or in terms of proportions or ratios, in various implementations.

4) Midline: A measure of the midline of the maxillary incisors, relative to the midline of the mandibular incisors. Techniques of this disclosure may measure the midline of the maxillary incisors, relative to the midline of the nose (if data about nose location is available).

5) Proximal Contacts: A measure of the size (area, volume, circumference, etc.) of the proximal contact between adjacent teeth. In the ideal circumstance, the teeth touch along the mesial/distal surfaces and the gums fill in gingivally to where the teeth touch. Black triangles may form if the gum tissue fails to fill the space below the proximal contact. In some instances, the size of the proximal contact may get progressively shorter for teeth located farther towards the posterior of the arch. In an ideal scenario, the proximal contact would be long enough so that there is an appropriately sized incisal embrasure and the gum tissue fills in the area below or gingival to the contact.

6) Embrasure: In some implementations, techniques of this disclosure may measure the size (area, volume, circumference, etc.) of an embrasure, the gap between teeth at either of the gingival or incisal edge. In some implementations, techniques of this disclosure may measure the symmetry between embrasures on opposite sides of the arch. An embrasure is based at least in part on the length of the length of the contact between teeth, and/or at least in part on the shape of the tooth. In some instances, the size of the embrasure may get progressively longer for teeth located farther towards the posterior of the arch.

Examples of Intra-tooth RDM are enumerated below, continuing with the numbering of other RDM listed above.

7) Length and/or Width: A measure of the length of a tooth relative to the width of that tooth. This metric may reveal, for example, that a patient has long central incisors. Width and length are defined as: a) width—mesial to distal distance; b) length—gingival to incisal distance; c) other dimensions of tooth body—the portions of tooth between the gingival region and the incisal edge. In some implementations, either or both of a length and a width may be measured for a tooth and compared to the length and/or width of one or more teeth.

8) Tooth Morphology: A measure of the primary anatomy of the tooth shape, such as line angles, buccal contours, and/or incisal angles and/or embrasures. The frequency and/or dimensions may be measured. In some implementations, the observed primary tooth shape aspects may be matched to one or more known styles. Techniques of this disclosure may measure secondary anatomy of the tooth shape, such as mamelon grooves. For instance, the frequency and/or dimensions may be measured. In some implementations, the observed secondary tooth shape aspects may be matched to one or more known styles. In some examples, techniques of this disclosure may measure tertiary anatomy of the tooth shape, such as perikymata or striations. For instance, the frequency and/or dimensions may be measured. In some implementations, the observed tertiary tooth shape aspects may be matched to one or more known styles.

9) Shade and/or Translucency: A measure of tooth shade and/or translucency. Tooth shade is often described by the Vita Classical or 3D Master shade guide. Tooth translucency is described by transmittance or a contrast ratio. Tooth shade and translucency may be evaluated (or measured) based on one or more of the following kinds of data pertaining to teeth: the incisal edge, incisal third, body and gingival third. The enamel layer translucency is general higher than the dentin or cementum layer. Shade and translucency may, in some implementations, be measured on a per-voxel (local) basis. Shade and translucency may, in some implementations, be measured on a per-area basis, such as an incisal area, tooth body area, etc. Tooth body may pertain to the portions of the tooth between the gingival region and the incisal edge.

10) Height of Contour: A measure of the contour of a tooth. When viewed from the proximal view, all teeth have a specific contour or shape, moving from the gingival aspect to the incisal. This is referred to as the facial contour of the tooth. In each tooth, there is a height of contour, where that shape is the most pronounced. This height of contour changes from the teeth in the anterior of the arch to the teeth in the posterior of the arch. In some implementations, this measurement may take the form of fitting against a template of known dimensions and/or known proportions. In some implementations, this measurement may quantify a degree of curvature along the facial tooth surface. In some implementations, measure the location along the contour of the tooth where the height of the curvature is most pronounced. This location may be measured as a distance away from the gingival margin or a distance away from the incisal edge, or a percentage along the length of the tooth.

Representation generation neural networks based on autoencoders, U-Nets, transformers, other types of encoder-decoder structures, convolution and/or pooling layers, or other models may benefit from the use of oral care arguments (e.g., oral care metrics or oral care parameters). For example, oral care metrics (e.g., orthodontic metrics or restoration design metrics) may convey aspects of the shape and/or structure of the patient's dentition (e.g., the shape and/or structure of an individual tooth, or the special relationships between two or more teeth) to the neural network models of this disclosure. Each oral care metric describes distinct information about the patient's dentition that may not be redundantly present in other input data that are provided to the neural network. For example, an “Overbite” metric may quantify the overlap between the upper and lower central incisors along the vertical Z-axis, information which may not otherwise, in some implementations, be readily ascertainable by a traditional neural network. Stated another way, the oral care metrics provide refined information about the patient's dentition that a traditional neural network (e.g., a representation generation neural network) may not be adequately trained or configured to extract. However, a neural network which is specifically trained to generate oral care metrics may overcome such a shortcoming, because, for example loss may be computed in such a way as to facilitate accurate oral care metrics prediction. Mesh oral care metrics may provide a processed version of the structure and/or shape of the patient's dentition, data which may not otherwise be available to the neural network. This processed information is often more accessible, or more amenable for encoding by the neural network. A system implementing the techniques disclosed herein has been utilized to run a number of experiments on 3D representations of teeth. For example, oral care metrics have been provided to a representation generation neural network which is based on a U-Net model. Based on experiments, it was found that systems using oral care metrics (e.g., “Overbite”, “Overjet” and “Canine Class Relationship” metrics) were at least 2.5% more accurate than systems that did not. Furthermore, training converges more quickly when the oral care metrics are used. Stated another way, the machine learning models trained using oral care metrics tended to be more accurate more quickly (at earlier epochs) than systems which did not. For an existing system observed to have a historical accuracy rate of 91%, an improvement in accuracy of 2.5% reduces the actual error rate by almost 30%.

PCT Application with Publication No. WO2020026117A1 is incorporated herein by reference in its entirety. WO2020026117A1 lists some examples of Orthodontic Metrics (OM). Further examples are disclosed herein. The orthodontic metrics may be used to quantify the physical arrangement of an arch of teeth for the purpose of orthodontic treatment (as opposed to restoration design metrics—which pertain to dentistry and describe the shape and/or form of one or more pre-restoration teeth, for the purpose of supporting dental restoration). These orthodontic metrics can measure how badly maloccluded the arch is, or conversely the metrics can measure how correctly arranged the teeth are. In some implementations, the GDL Setups model (or RL Setups, VAE Setups, Capsule Setups, MLP Setups, Diffusion Setups, PT Setups, Similarity Setups and FDG Setups) may incorporate one or more of these orthodontic metrics, or other similar or related orthodontic metrics. In some implementations, such orthodontic metrics may be incorporated into the feature vector for a mesh element, where these per-element feature vectors are provided to the setups prediction network as inputs. In some implementations, such orthodontic metrics may be directly consumed by a generator, an MLP, a transformer, or other neural network as direct inputs (such as presented in one or more input vectors of real numbers S, such as described elsewhere in this disclosure. The use of such orthodontic metrics in the training of the generator may improve the performance (i.e., correctness) of the resulting generator, resulting in predicted transforms which place teeth more nearly in the correct final setups poses than would otherwise be possible. Such orthodontic metrics may be consumed by an encoder structure or by a U-Net structure (in the case of GDL Setups). Such orthodontic metrics may be provided by an autoencoder, variational autoencoder, masked autoencoder or regularized autoencoder (in the case of the VAE Setups, VAE Mesh Element Labelling, MAE Mesh In-Filling). Such orthodontic metrics may be consumed by a neural network which generates action predictions as a part of a reinforcement learning RL Setups model. Such orthodontic metrics may be consumed by a classifier which applies a label to a setup arch (e.g., labels such as mal, staging or final setup). This description is non-limiting, as the orthodontic metrics may also be incorporated in other ways into the various techniques of this disclosure.

The various loss calculations of the present disclosure may, in some examples, incorporate one or more orthodontic metrics, with the advantage of improving the correctness of the resulting neural network. An orthodontic metric may be used to directly compare a predicted example to the corresponding ground truth example (such as is done with the metrics in the Setups Comparison description). In other examples, one or more orthodontic metrics may be taken from this section and incorporated into a loss computation. Such an orthodontic metric may be computed on the predicted example, and then the orthodontic metric would also be computed on the ground truth example. These two orthodontic metrics results would then be consumed by the loss computation, with the advantage of improving the performance of the resulting neural network. In some implementations, one or more orthodontic metrics pertaining to the alignment of two or more adjacent teeth may be computed and incorporated into a loss function, for example, to train, at least in part, a setups prediction neural network. In some implementations, such an orthodontic metric may influence the network to align the mesial surface of a tooth with the distal surface of an adjacent tooth. Backpropagation is an example algorithm by which a neural network may be trained using one or more loss values.

In some implementations, one or more orthodontic metrics may be used to evaluate the predicted output of a neural network, such as a setups prediction. Such a metric(s) may enable the training algorithm to determine how close the predicted output is to an acceptable output, for example, in a quantified sense. In some implementations, this use of an orthodontic metric may enable a loss value to be computed which does not depend entirely on a comparison to a ground truth. In some implementations, such a use of an orthodontic metric may enable loss calculation and network training to proceed without the need for a comparison against a ground truth example. The advantage of such an approach is that loss may be computed based on a general principle or specification for the predicted output (such as a setup) rather than tying loss calculation to a specific ground truth example (which may have been defined by a particular doctor, clinician, or technician, whose treatment philosophy may differ from that of other technicians or doctors). In some implementations, such an orthodontic metric may be defined based on a FID (Frechet Inception Distance) score.

The following is a description of some of the orthodontic metrics which are used to quantify the state of a set of teeth in an arch for the purpose of orthodontic treatment. These orthodontic metrics indicate the degree of malocclusion that the teeth are in at a given stage of clear tray aligner treatment.

An orthodontic metric that can be computed using tensors may be especially advantageous when training one of the neural networks of the present disclosure, because tensor operations may promote efficient computations. The more efficient (and faster) the computation, the faster the rate at which training can proceed.

In some examples, an error pattern may be identified in one or more predicted outputs of an ML model (e.g., a transformation matrix for a predicted tooth setup, a labelling of mesh elements for mesh cleanup, an addition of mesh elements to a mesh for the purpose of mesh in-filling, a classification label for a setup, a classification label for a tooth mesh, etc.). One or more orthodontic metrics may be selected to become an input to the next round of ML model training, to address any pattern of errors or deficiencies which may be identified in the one or more predicted outputs.

Some OM may be defined relative to an archfrom coordinate frame, the LDE coordinate system. In some implementations, a point may be described using an LDE coordinate frame relative to an archform, where L, D and E correspond to: 1) Length along the curve of the archform, 2) Distance away from the archform, and 3) distance in the direction perpendicular to the L and D axes (which may be termed Eminence), respectively.

Various of the OM and other techniques of the present disclosure may compute collisions between 3D representations (e.g., of oral care objects, such as teeth). Such collisions may be computed as at least one of: 1) penetration distance between 3D tooth representations, 2) count of overlapping mesh elements between 3D tooth representations, and 3) volume of overlap between 3D tooth representations. In some implementations, an OM may be defined to quantify the collision of two or more 3D representations of oral care structures, such as teeth. Some optimization algorithms, such as setups prediction techniques, may seek to minimize collisions between oral care structures (such as teeth). Between-arch orthodontic metrics are as follows.

Six (6) metrics for the comparison of two or more arches are listed below. Other suitable comparison orthodontic metrics are found elsewhere in this disclosure, such as in the section for the Setups Comparison technique.
1. Rotation geodesic distance (rotation between predicted example and ground truth setup example)
2. Translation distance (gap between predicted example and ground truth setup example)
3. Normalized translation distance
4. 3D alignment error that measures the distance between predicted mesh elements and ground truth mesh elements, in units of mm.
5. Normalized 3D alignment
6. Percent overlap (% overlap) by volume (alternatively % overlap by mesh elements) of predicted example and corresponding ground truth example

Within-arch orthodontic metrics are as follows.

Alignment—A 3D tooth orientation vector may be calculated using the tooth's mesial-distal axis. A 3D vector, which may be tangent vector to the archform at the position of the tooth may also be calculated. The XY components (i.e., which may be 2D vectors) may then be used to compare the orientation of the archform at the tooth's location to the tooth's orientation in XY space. Cosine similarity may be used to calculate the 2D orientation difference (angle) between the archform tangent and the tooth's mesial-distal axis.
Arch Symmetry—For each left-right pair of teeth (e.g., lower left lateral incisor and/or lower right lateral incisor) the absolute difference may be calculated between each tooth's X-coordinate and the global coordinate reference frame's X-axis. This delta may indicate the arch asymmetry for a given tooth pair. The result of such a calculation may be the mean X-axis delta of one or more tooth-pairs from the arch. This calculation may, in some implementations, be performed relative to the Y-axis with y-coordinates (and/or relative to the Z axis with Z-coordinates).
Archform D-axis Differences—May compute the D dimension difference (i.e., the positional difference in the facial-lingual direction) between two arch states, for one or more teeth. May, in some implementations, return a dictionary of the D-direction tooth movement for each tooth, with tooth UNS number as the key. May use the LDE coordinate system relative to an archform.
Archform (Lower) Length Ratio—May compute the ratio between the current lower arch length and the arch length as it was in the original maloccluded lower arch.
Archform (Upper) Length Ratio—May compute the ratio between the current upper arch length and the arch length as it was in the original maloccluded upper arch.
Archform Parallelism (Full arch)—For at least one local tooth coordinate system origin in the upper arch, the one or more nearest origins (e.g., tooth local coordinate system origins) in the lower arch. In some implementations, the two nearest origins may be used. May compute the straight line distance from the upper arch point to the line formed between the origins of the two teeth in the opposing (lower) arch. May return the standard deviation of the set of “point-to-line” distances mentioned above, where the set may be composed of the point-to-line distances for each tooth in the arch.
Archform Parallelism (Individual tooth)—This metric may share some computational elements with the archform_parallelism_global orthodontic metric, except that this metric may input the mean distance from a tooth origin to the line formed by the neighboring teeth in opposing arches (e.g., a tooth in the upper arch and the corresponding tooth in the lower arch). The mean distance may be computed for one or more such pairs of teeth. In some implementations, this may be computed for all pairs of teeth. Then the mean distance may be subtracted from the distance that is computed for each tooth pair. This OM may yield the deviation of a tooth from a “typical” tooth parallelism in the arch.
Buccolingual Inclination—For at least one molar or premolar, find the corresponding tooth on the opposite side of the same arch (i.e., for a tooth on the left side of the arch, find the same type of tooth on the right side and vice versa). This OM may compute an n-element list for each tooth (e.g. n may equal 2). This list may contain at least the tooth IDs of the teeth in each pair of teeth (e.g., LeftLowerFirstMolar and RightLowerFirstMolar in a list=[left_tooth_idx_1, right tooth idx_2]). Such an n-element vector may be computed for each molar and each premolar in the upper and lower arches. The buccal cusps maybe identified on the molars and premolars on each of the left and right sides of the arch. Draw a line between the buccal cusps of the left tooth and the buccal cusps on the right tooth. Make a plane using this line and the z-axis of the arch. The lingual cusps may be projected onto the plane (i.e., at this point the angle of inclination may be determined). By performing an additional projection, the approximate vertical distance between the lingual cusps and the buccal cusps may be computed. This distance may be used as the buccolingual inclination OM.
Canine Overbite—The upper and lower canines may be identified. The first premolar for the given side of the mouth may be identified. On a given side of the arch, a distance may be computed between the upper canine and the lower canine, and also between the upper pre-molar and the lower pre-molar. The average (or median, or mode or some other statistic) may be computed for the measured distances. The z-component of this result indicates the degree of overbite. Overbite may be computed between any tooth in one arch and the corresponding tooth in the other arch.
Canine Overjet Contact—May calculate the collisions (e.g., collision distances) between pairs of canines on opposing arches.
Canine Overjet Contact KDE—May take an orthodontic metric score for the current patient case as input, and may convert that score into to a log-likelihood using a previously trained kernel density estimation (KDE) model or distribution. This operation may yield information about where in the distribution of “typical” values this patient case lies.
Canine Overjet—This OM may share some computational steps with the canine overbite OM. In some implementations, average distances may be computed. In some implementations, the distance calculation may compute the Euclidean distance of the XY components of a tooth in the upper arch and a tooth in the lower arch, to yield overjet (i.e., as opposed to computing the difference in Z-components, as may be performed for canine overbite). Overjet may be computed between any tooth in one arch and the corresponding tooth in the other arch.
Canine Class Relationship (also applies to first, second and third molars)—This OM may, in some implementations comprise two functions (e.g., written in Python).
get_canine_landmarks( ): Get landmarks for each tooth which may be used to compute the class relationship, and then, in some implementations, map those landmarks onto the global coordinate space so that measurements may be made between teeth.
class_relationship_score_by_side( ): May compute the average position of at least one landmark on at least one tooth in the lower arch, and may compute the same for the upper arch. Then may compute the vector from the upper arch landmark position to the lower arch landmark position, and finally projects this vector onto the lower arch to yield a quantification (e.g., as a scalar) of the amount of delta in “arch1-axis” position there is. This OM may compute how far forward or behind the tooth is positioned on the 1-axis relative to the tooth or teeth of interest in the opposing arch.
   Crossbite—Fossa in at least one upper molar may be located by finding the halfway point between distal and mesial marginal ridge saddles of the tooth. A lower molar cusp may lie between the marginal ridges of the corresponding upper molar. This OM may compute a vector from the upper molar fossa midpoint to the lower molar cusp. This vector may be projected onto the d-axis of the archform, yielding a lateral measure of distance from the cusp to the fossa. This distance may define the crossbite magnitude.
   Edge Alignment—This OM may identify the leftmost and rightmost edges of a tooth, and may identify the same for that tooth's neighbor.
The OM may then draw a vector from the leftmost edge of the tooth to the leftmost edge of the tooth's neighbor.
The OM may then draw a vector from the rightmost edge of the tooth to the rightmost edge of the tooth's neighbor.
The OM may then calculates the linear fit error between the two vectors.
Such a calculation may involve making two vectors:

$$Vec\_tooth = right\_tooths \_leftside to left\_tooths \_leftside \\ Vec\_neighbor = right\_tooths \_rightside to left\_tooths \_leftside$$

And then may involve computing the dot-product of these two vectors and subtracting the result from 1. (i.e., EdgeAlignment score=1−abs(dot(Vec_tooth, Vec_neighbor))).
A score of 0 may indicate perfect alignment. A score of 1 may mean perpendicular alignment.
   Incisor Interarch Contact KDE—May identify the deviation of the IncisorInterarchContact from the mean of a modeled distribution of such statistics across a dataset of one or more other patient cases.
   Leveling—May compute a measure of leveling between a tooth and its neighbor. This OM may calculate the difference in height between two or more neighboring teeth. For molars, this OM may use the midpoint between the mesial and distal saddle ridges as the height of the molar. For non-molar teeth, this OM may use the length of the crown from gums to tip. In some implementations, the tip may be the origin of the local coordinate space of the tooth. Other implementations may place the origin in other locations. A simple subtraction between the heights of neighboring teeth may yield the leveling delta between the teeth (e.g., by comparing Z components).
   Midline—May compute the position of the midline for the upper incisors and/or the lower incisors, and then may compute the distance between them.
   Molar Interarch Contact KDE—May compute a molar interarch contact score (i.e., a collision depth or other type of collision), and then may identify where that score lies in a pre-defined KDE (distribution) built from representative cases.
   Occlusal Contacts—For a particular tooth from the arch, this OM may identify one or more landmarks (e.g., mesial cusp, or central cusp, etc.). Get the tooth transform for that tooth. For each cusp on the current tooth, the cusp may be scored according to how well the cusp contacts the neighboring (corresponding) tooth in the opposite arch. A vector may be found from the cusp of the tooth in question to the vertical intersection point in the corresponding tooth of the opposing arch. The distance and/or direction (i.e., up or down) to the opposing arch may be computed. A list may be returned that contains the resulting signed distances, one for each cusp on the tooth in question.
   Overbite—The upper and lower central incisors may be compared along the z-axis. The difference along the z-axis may be used as the overbite score.
   Overjet—The upper and lower central incisors may be compared along the y-axis. The difference along the y-axis may be used as the overjet score.
   Molar Interarch Contact—May calculate the contact score between molars and may use collision measurement(s) (such as collision depth).
   Root Movement d—The tooth transforms for an initial state and a next state may be received. The archform axes at a point L along the archform may be computed. This OM may return a distance moved along the d-axis. This may be accomplished by projecting the root pivot point onto the d-axis.
   Root Movement 1—The tooth transforms for an initial state and a next state may be received. The archform axes at a point L along the archform may be computed. This OM may return a distance moved along the 1-axis. This may be accomplished by projecting the root pivot point onto the 1-axis.
   Spacing—May compute the spacing between each tooth and its neighbor. The transforms and meshes for the arch may be received. The left and right edges of each tooth mesh may be computed. One or more points of interest may be transformed from local coordinates into the global arch coordinate frame. The spacing may be computed in a plane (e.g., the XY plane) between each tooth and its neighbor to the “left”. May return an array of one or more Euclidean distances (e.g., such as in the XY plane) which may represent the spacing between each tooth and its neighbor to the left.
   Torque—May compute torque (i.e., rotation around and axis, such as the x-axis). For one or more teeth, one or more rotations may be converted from Euler angles into one or more rotation matrices. A component (such as a x-component) of the rotations may be extracted and converted back into Euler angles. This x-component may be interpreted as the torque for a tooth. A list may be returned which contains the torque for one or more teeth and may be indexed by the UNS number of the tooth.

The neural networks of this disclosure may exploit one or more benefits of the operation of parameter tuning, whereby the inputs and parameters of a neural network are optimized to produce more data-precide results. One parameter which may be tuned is neural network learning rate (e.g., which may have values such as 0.1, 0.01, 0.001, etc.). Data augmentation schemes may also be tuned or optimized, such as schemes where “shiver” is added to the tooth meshes before being input to the neural network (i.e., small random rotations, translations and/or scaling may be applied to vary the dataset and make the neural network robust to variations in data).

A subset of the neural network model parameters available for tuning are as follows:
Learning rate (LR) decay rate (e.g., how much the LR decays during a training run)
Learning rate (LR). The floating-point value (e.g., 0.001) that is used by the optimizer.
LR schedule (e.g., cosine annealing, step, exponential)
Voxel size (for cases with sparse mesh processing operations)
Dropout % (e.g., dropout which may be performed in a linear encoder)
LR decay step size (e.g., decay every 10 or 20 or 30 epochs)
Model scaling, which may increase or decrease the count of layers and/or the count of parameters per layer.

Parameter tuning may be advantageously applied to the training of a neural network for the prediction of final setups or intermediate staging to provide data precision-oriented technical improvements. Parameter tuning may also be advantageously applied to the training of a neural network for mesh element labeling or a neural network for mesh in-filling. In some examples, parameter tuning may be advantageously applied to the training of a neural network for tooth reconstruction. In terms of classifier models of this disclosure, parameter tuning may be advantageously applied to a neural network for the classification of one or more setups (i.e., classification of one or more arrangements of teeth). The advantage of parameter tuning is to improve the data precision of the output of a predictive model or a classification model. Parameter tuning may, in some instances, provide the advantage of obtaining the last remaining few percentage points of validation accuracy out of a predictive or classification model.

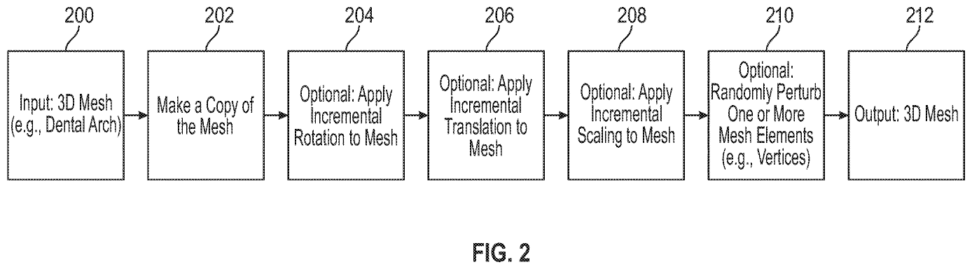

Various neural network models of this disclosure may draw benefits from data augmentation. Examples include models of this which are trained on 3D meshes, such as GDL Setups, RL Setups, VAE Setups, Capsule Setups, MLP Setups, Diffusion Setups, PT Setups, Similarity Setups, FDG Setups, Setups Classification, Setups Comparison, VAE Mesh Element Labeling, MAE Mesh In-filling, Mesh Reconstruction VAE, and Validation Using Autoencoders. FIG. 2 is a method illustrating a data augmentation method of this disclosure. Data augmentation, such as by way of the method shown in FIG. 2, may increase the size of the training dataset of dental arches. Data augmentation can provide additional training examples by adding random rotations, translations, and/or rescaling to copies of existing dental arches. In some implementations of the techniques of this disclosure, data augmentation may be carried out by perturbing or jittering the vertices of the mesh, in a manner similar to that described in (“Equidistant and Uniform Data Augmentation for 3D Objects”, IEEE Access, Digital Object Identifier 10.1109/ACCESS.2021.3138162). The position of a vertex may be perturbed through the addition of Gaussian noise, for example with zero mean, and 0.1 standard deviation. Other mean and standard deviation values are possible in accordance with the techniques of this disclosure.

FIG. 2 shows a data augmentation method that systems of this disclosure may apply to 3D oral care representations. A non-limiting example of a 3D oral care representation is a tooth mesh or a set of tooth meshes. Tooth data 200 (e.g., 3D meshes) are received at the input. The systems of this disclosure may generate copies of the tooth data 200 (202). In the example of FIG. 2, the systems of this disclosure may apply one or more stochastic rotations to the tooth data 200 (204). In the example of FIG. 2, the systems of this disclosure may apply stochastic translations to the tooth data 200 (206). The systems of this disclosure may apply stochastic scaling operations to the tooth data 200 (208). The systems of this disclosure may apply stochastic perturbations to one or more mesh elements of the tooth data 200 (210). The systems of this disclosure may output augmented tooth data 212 that are formed by way of the method of FIG. 2.

Some techniques of the present disclosure, for example the setups comparison technique, and the setups prediction techniques (e.g., such as GDL Setups, MLP Setups, VAE Setups and the like), may benefit from a processing step which may align (or register) arches of teeth (e.g., where a tooth may be represented by a 3D point cloud, or some other type of 3D representation described herein). Such a processing setup may, for example, be used to register a ground truth setup arch from a patient case with the maloccluded arch from that same case, before these mal and ground truth setup arches are used to train a setups prediction neural network model. Such a step may aid in loss calculation, because the predicted arch (e.g., an arch outputted by a generator) may be in better alignment with the ground truth setup arch, a condition which may facilitate the calculation of reconstruction loss, representation loss, L1 loss, L2 loss, MSE loss and/or other kinds of losses described herein. In some implementations, an iterative closest point (ICP) technique may be used for such registration. ICP may minimize the squared errors between corresponding entities, such as 3D representations. In some implementations, linear least squares calculations may be performed. In some implementations, non-linear least squares calculations may be performed. Various registration models may incorporate portions of the following algorithms, in whole or in part: Levenberg-Marquardt ICP, Least Square Rigid transformation, Robust Rigid transformation, random sample consensus (RANSAC) ICP, K-means based RANSAC ICP and Generalized ICP (GICP). Registration may, in some instances, help decrease the subjectivity and/or randomness that may, in some instances, occur in reference ground truth setup designs which have been designed by technicians (i.e., two technicians may produce different but valid final setups outputs for the same case) or by other optimization techniques.

Because generator networks of this disclosure can be implemented as one or more neural networks, the generator may contain an activation function. When executed, an activation function outputs a determination of whether or not a neuron in a neural network will fire (e.g., send output to the next layer). Some activation functions may include binary step functions, or linear activation functions. Other activation functions impart non-linear behavior to the network, including sigmoid/logistic activation functions, Tanh (hyperbolic tangent) functions, rectified linear units (ReLU), leaky ReLU functions, parametric ReLU functions, exponential linear units (ELU), softmax function, swish function, Gaussian error linear unit (GELU), or scaled exponential linear unit (SELU). A linear activation function may be well suited to some regression applications (among other applications), in an output layer. A sigmoid/logistic activation function may be well suited to some binary classification applications (among other applications), in an output layer. A softmax activation function may be well suited to some multiclass classification applications (among other applications), in an output layer. A sigmoid activation function may be well suited to some multilabel classification applications (among other applications), in an output layer. A ReLU activation function may be well suited in some convolutional neural network (CNN) applications (among other applications), in a hidden layer. A Tanh and/or sigmoid activation function may be well suited in some recurrent neural network (RNN) applications (among other applications), for example, in a hidden layer. There are multiple optimization algorithms which can be used in the training of the neural networks of this disclosure (such as in updating the neural network weights), including gradient descent (which determines a training gradient using first-order derivatives and is commonly used in the training of neural networks), Newton's method (which may make use of second derivatives in loss calculation to find better training directions than gradient descent, but may require calculations involving Hessian matrices), and conjugate gradient methods (which may yield faster convergence than gradient descent, but do not require the Hessian matrix calculations which may be required by Newton's method). In some implementations, additional methods may be employed to update weights, in addition to or in place of the techniques described above. These additional methods include the Levenberg-Marquardt method and/or simulated annealing. The backpropagation algorithm is used to assign the results of loss calculation back into the network so that network weights can be adjusted, and learning can progress.

Neural networks contribute to the functioning of many of the applications of the present disclosure, including but not limited to: GDL Setups, RL Setups, VAE Setups, Capsule Setups, MLP Setups, Diffusion Setups, PT Setups, Similarity Setups, Tooth Classification, Setups Classification, Setups Comparison, VAE Mesh Element Labeling, MAE Mesh In-filling, Mesh Reconstruction Autoencoder, Validation Using Autoencoders, imputation of oral care parameters, 3D mesh segmentation (3D representation segmentation), Coordinate System Prediction, Mesh Cleanup, Restoration Design Generation, Appliance Component Generation and/or Placement, or Archform Prediction. The neural networks of the present disclosure may embody part or all of a variety of different neural network models. Examples include the U-Net architecture, multi-later perceptron (MLP), transformer, pyramid architecture, recurrent neural network (RNN), autoencoder, variational autoencoder, regularized autoencoder, conditional autoencoder, capsule network, capsule autoencoder, stacked capsule autoencoder, denoising autoencoder, sparse autoencoder, conditional autoencoder, long/short term memory (LSTM), gated recurrent unit (GRU), deep belief network (DBN), deep convolutional network (DCN), deep convolutional inverse graphics network (DCIGN), liquid state machine (LSM), extreme learning machine (ELM), echo state network (ESN), deep residual network (DRN), Kohonen network (KN), neural Turing machine (NTM), or generative adversarial network (GAN). In some implementations, an encoder structure or a decoder structure may be used. Each of these models provides one or more of its own particular advantages. For example, a particular neural networks architecture may be especially well suited to a particular ML technique. For example, autoencoders are particularly suited to the classification of 3D oral care representations, due to the ability to encode the 3D oral care representation into a form which is more easily classifiable.

In some implementations, the neural networks of this disclosure can be adapted to operate on 3D point cloud data (alternatively on 3D meshes or 3D voxelized representation). Numerous neural network implementations may be applied to the processing of 3D representations and may be applied to training predictive and/or generative models for oral care applications, including: PointNet, PointNet++, SO-Net, spherical convolutions, Monte Carlo convolutions and dynamic graph networks, PointCNN, ResNet, MeshNet, DGCNN, VoxNet, 3D-ShapeNets, Kd-Net, Point GCN, Grid-GCN, KCNet, PD-Flow, PU-Flow, MeshCNN and DSG-Net. Oral care applications include, but are not limited to: setups prediction (e.g., using VAE, RL, MLP, GDL, Capsule, Diffusion, etc. which have been trained for setups prediction), 3D representation segmentation, 3D representation coordinate system prediction, element labeling for 3D representation clean-up (VAE for Mesh Element labeling), in-filling of missing elements in 3D representation (MAE for Mesh In-Filling), dental restoration design generation, setups classification, appliance component generation and/or placement, archform prediction, imputation of oral care parameters, setups validation, or other validation applications and tooth 3D representation classification.

Some implementations of the techniques of this disclosure incorporate the use of an autoencoder. Autoencoders that can be used in accordance with aspects of this disclosure include but are not limited to: AtlasNet, FoldingNet and 3D-PointCapsNet. Some autoencoders may be implemented based on PointNet.

Representation learning may be applied to setups prediction techniques of this disclosure by training a neural network to learn a representation of the teeth, and then using another neural network to generate transforms for the teeth. Some implementations may use a VAE or a Capsule Autoencoder to generate a representation of the reconstruction characteristics of the one or more meshes related to the oral care domain (including, in some instances, information about the structures of the tooth meshes). Then that representation (either a latent vector or a latent capsule) may be used as input to a module which generates the one or more transforms for the one or more teeth. These transforms may in some implementations place the teeth into final setups poses. These transforms may in some implementations place the teeth into intermediate staging poses. In some implementations, a transform may be described by a 9×1 transformation vector (e.g., that specifies a translation vector and a quaternion). In other implementations, a transform may be described by a transformation matrix (e.g., a 4×4 affine transformation matrix).

In some implementations, systems of this disclosure may implement a principal components analysis (PCA) on an oral care mesh and use the resulting principal components as at least a portion of the representation of the oral care mesh in subsequent machine learning and/or other predictive or generative processing.

An autoencoder may be trained to generate a latent form of a 3D oral care representation. An autoencoder may contain a 3D encoder (which encodes a 3D oral care representation into a latent form), and/or a 3D decoder (which reconstructs that latent from into a facsimile of the inputted 3D oral care representation). Although this disclosure refers to 3D encoders and 3D decoders, the term 3D should be interpreted in a non-limiting fashion to encompass multi-dimensional modes of operation. For example, systems of this disclosure may train multi-dimensional encoders and/or multi-dimensional decoders.

Systems of this disclosure may implement end-to-end training. Some of the end-to-end training-based techniques of this disclosure may involve two or more neural networks, where the two or more neural networks are trained together (i.e., the weights are updated concurrently during the processing of each batch of input oral care data). End-to-end training may, in some implementations, be applied to setups prediction by concurrently training a neural network which learns a representation of the teeth, along with a neural network which generates the tooth transforms.

According to some of the transfer learning-based implementations of this disclosure, a neural network (e.g., a U-Net) may be trained on a first task (e.g., such as coordinate system prediction). The neural network trained on the first task may be executed to provide one or more of the starting neural network weights for the training of another neural network that is trained to perform a second task (e.g., setups prediction). The first network may learn the low-level neural network features of oral care meshes and be shown to work well at the first task. The second network may exhibit faster training and/or improved performance by using the first network as a starting point in training. Certain layers may be trained to encode neural network features for the oral care meshes that were in the training dataset. These layers may thereafter be fixed (or be subjected to minor changes over the course of training) and be combined with other neural network components, such as additional layers, which are trained for one or more oral care tasks (such as setups prediction). In this manner, a portion of a neural network for one or more of the techniques of the present disclosure (e.g., setups prediction) may receive initial training on another task, which may yield important learning in the trained network layers. This encoded learning may then be built upon with further task-specific training of another network.

In accordance with this disclosure, transfer learning may be used for setups prediction, as well as for other oral care applications, such as mesh classification (e.g., tooth or setups classification), mesh element labeling, mesh element in-filling, procedure parameter imputation, mesh segmentation, coordinate system prediction, restoration design generation, mesh validation (for any of the applications disclosed herein). In some implementations, a neural network trained to output predictions based on oral care meshes may first be partially trained on one of the following publicly available datasets, before being further trained on oral care data: Google PartNet dataset, ShapeNet dataset, ShapeNetCore dataset, Princeton Shape Benchmark dataset, ModelNet dataset, ObjectNet3D dataset, Thingi10K dataset (which is especially relevant to 3D printed parts validation), ABC: A Big CAD Model Dataset For Geometric Deep Learning, ScanObjectNN, VOCASET, 3D-FUTURE, MCB: Mechanical Components Benchmark, PoseNet dataset, PointCNN dataset, MeshNet dataset, MeshCNN dataset, PointNet++ dataset, PointNet dataset, or PointCNN dataset.

In some implementations, a neural network which was previously trained on a first dataset (either oral care data or other data) may subsequently receive further training on oral care data and be applied to oral care applications (such as setups prediction). Transfer learning may be employed to further train any of the following networks: GCN (Graph Convolutional Networks), PointNet, ResNet or any of the other neural networks from the published literature which are listed above.

In some implementations, a first neural network may be trained to predict coordinate systems for teeth (such as by using the techniques described in WO2022123402A1 or US Provisional Application No. U.S. 63/366,492). A second neural network may be trained for setups prediction, according to any of the setups prediction techniques of the present disclosure (or a combination of any two or more of the techniques described herein). Transfer learning may assign at least a portion of the knowledge or capability of the first neural network to the second neural network. As such, transfer learning may provide the second neural network an accelerated training phase to reach convergence. In some implementations, the training of the second network may, after being augmented with the transferred learning, then be completed using one or more of the techniques of this disclosure.

Systems of this disclosure may train ML models with representation learning. The advantages of representation learning include that the generative network (e.g., neural network that predicts a transform for use in setups prediction) can be configured to receive input with a known size and/or standard format, as opposed to receiving input with a variable size or structure. Representation learning may produce improved performance over other techniques, because noise in the input data may be reduced (e.g., because the representation generation model extracts hierarchical neural network features and/or reconstruction characteristics of an inputted representation (e.g., a mesh or point cloud) through loss calculations or network architectures chosen for that purpose).

Reconstruction characteristics may comprise values in of a latent representation (e.g., a latent vector) that describe aspects of the shape and/or structure of the 3D representation that was provided to the representation generation module that generated the latent representation. The weights of the encoder module of a reconstruction autoencoder, for example, may be trained to encode a 3D representation (e.g., a 3D mesh, or others described herein) into a latent vector representation (e.g., a latent vector). Stated another way, the capability to encode a large set (e.g., hundreds, thousands or millions) of mesh elements into a latent vector (e.g., of hundreds or a thousand real values—e.g., 512, 1024, etc.) may be learned by the weights of the encoder. Each dimension of that latent vector may contain a real number which describes some aspect of the shape and/or structure of the original 3D representation. The weights of the decoder module of the reconstruction autoencoder may be trained to reconstruct the latent vector into a close facsimile of the original 3D representation. Stated another way, the capability to interpret the dimensions of the latent vector, and to decode the values within those dimensions, may be learned by the decoder. In summary, the encoder and decoder neural network modules are trained to perform the mapping of a 3D representation into a latent vector, which may then be mapped back (or otherwise reconstructed) into a 3D representation that is substantially similar to an original 3D representation for which the latent vector was generated.

Returning to loss calculation, examples of loss calculation may include KL-divergence loss, reconstruction loss or other losses disclosed herein. Representation learning may reduce the size of the dataset required for training a model, because the representation model learns the representation, enabling the generative network to focus on learning the generative task. The result may be improved model generalization because meaningful neural network features of the input data (e.g., local and/or global features) are made available to the generative network. Stated another way, a first network may learn the representation, and a second network may make the predictive decision. By training two networks to perform their own separate tasks, each of the networks may generate more accurate results for their respective tasks than with a single network which is trained to both learn a representation and make a decision. In some instances, transfer learning may first train a representation generation model. That representation generation model (in whole or in part) may then be used to pre-train a subsequent model, such as a generative model (e.g., that generates transform predictions). A representation generation model may benefit from taking mesh element features as input, to improve the capability of a second ML module to encode the structure and/or shape of the inputted 3D oral care representations in the training dataset.

One or more of the neural networks models of this disclosure may have attention gates integrated within. Attention gate integration provides the enhancement of enabling the associated neural network architecture to focus resources on one or more input values. In some implementations, an attention gate may be integrated with a U-Net architecture, with the advantage of enabling the U-Net to focus on certain inputs, such as input flags which correspond to teeth which are meant to be fixed (e.g., prevented from moving) during orthodontic treatment (or which require other special handling). An attention gate may also be integrated with an encoder or with an autoencoder (such as VAE or capsule autoencoder) to improve predictive accuracy, in accordance with aspects of this disclosure. For example, attention gates can be used to configure a machine learning model to give higher weight to aspects of the data which are more likely to be relevant to correctly generated outputs. As such, and because a machine learning model configured with these attention gates (or mechanisms) utilizes aspects of the data that are more likely to be relevant to correctly generated outputs, the ultimate predictive accuracy of those machine learning models is improved.

The quality and makeup of the training dataset for a neural network can impact the performance of the neural network in its execution phase. Dataset filtering and outlier removal can be advantageously applied to the training of the neural networks for the various techniques of the present disclosure (e.g., for the prediction of final setups or intermediate staging, for mesh element labeling or a neural network for mesh in-filling, for tooth reconstruction, for 3D mesh classification, etc.), because dataset filtering and outlier removal may remove noise from the dataset. And while the mechanism for realizing an improvement is different than using attention gates, that ultimate outcome is that this approach allows for the machine learning model to focus on relevant aspects of the dataset and may lead to improvements in accuracy similar to improvements in accuracy realized vis-à-vis attention gates.

In the case of a neural network configured to predict a final setup, a patient case may contain at least one of a set of segmented tooth meshes for that patient, a mal transform for each tooth, and/or a ground truth setup transform for each tooth. In the case of a neural network to predict a set of intermediate stage setups, a patient case may contain at least one of a set of segmented tooth meshes for that patient, a mal transform for each tooth, and/or a set of ground truth intermediate stage transforms for each tooth. In some implementations, a training dataset may exclude patient cases which contact passive stages (i.e., stages where the teeth of an arch do not move). In some implementations, the dataset may exclude cases where passive stages exist at the end of treatment. In some implementations, a dataset may exclude cases where overcrowding is present at the end of treatment (i.e., where the oral care provider, such as an orthodontist or dentist) has chosen a final setup where the tooth meshes overlap to some degree. In some implementations, the dataset may exclude cases of a certain level (or levels) of difficulty (e.g., easy, medium and hard).

In some implementations, the dataset may include cases with zero pinned teeth (or may include cases where at least one tooth is pinned). A pinned tooth may be designated by a technician as they design the treatment to stop the various tools from moving that particular tooth. In some implementations, a dataset may exclude cases without any fixed teeth (conversely, where at least one tooth is fixed). A fixed tooth may be defined as a tooth that shall not move in the course of treatment. In some implementations, a dataset may exclude cases without any pontic teeth (conversely, cases in which at least one tooth is pontic). A pontic tooth may be described as a “ghost” tooth that is represented in the digital model of the arch but is either not actually present in the patient's dentition or where there may be a small or partial tooth that may benefit from future work (such as the addition of composite material through a dental restoration appliance). The advantage of including a pontic tooth in a patient's case is to leave space in the arch as a part of a plan for the movements of other teeth, in the course of orthodontic treatment. In some instances, a pontic tooth may save space in the patient's dentition for future dental or orthodontic work, such as the installation of an implant or crown, or the application of a dental restoration appliance, such as to add composite material to an existing tooth that is too small or has an undesired shape.

In some implementations, the dataset may exclude cases where the patient does not meet an age requirement (e.g., younger than 12). In some implementations, the dataset may exclude cases with interproximal reduction (IPR) beyond a certain threshold amount (e.g., more than 1.0 mm). The dataset to train a neural network to predict setups for clear tray aligners (CTA) may exclude patient cases which are not related to CTA treatment. The dataset to train a neural network to predict setups for an indirect bonding tray product may exclude cases which are not related to indirect bonding tray treatment. In some implementations, the dataset may exclude cases where only certain teeth are treated. In such implementations, a dataset may comprise of only cases where at least one of the following are treated: anterior teeth, posterior teeth, bicuspids, molars, incisors, and/or cuspids.

The mesh comparison module may compare two or more meshes, for example for the computation of a loss function or for the computation of a reconstruction error. Some implementations may involve a comparison of the volume and/or area of the two meshes. Some implementations may involve the computation of a minimum distance between corresponding vertices/faces/edges/voxels of two meshes. For a point in one mesh (vertex point, mid-point on edge, or triangle center, for example) compute the minimum distance between that point and the corresponding point in the other mesh. In the case that the other mesh has a different number of elements or there is otherwise no clear mapping between corresponding points for the two meshes, different approaches can be considered. For example, the open-source software packages CloudCompare and MeshLab each have mesh comparison tools which may play a role in the mesh comparison module for the present disclosure. In some implementations, a Hausdorff Distance may be computed to quantify the difference in shape between two meshes. The open-source software tool Metro, developed by the Visual Computing Lab, can also play a role in quantifying the difference between two meshes. The following paper describes the approach taken by Metro, which may be adapted by the neural networks applications of the present disclosure for use in mesh comparison and difference quantification: “Metro: measuring error on simplified surfaces” by P. Cignoni, C. Rocchini and R. Scopigno, Computer Graphics Forum, Blackwell Publishers, vol. 17(2), June 1998, pp 167-174.

Some techniques of this disclosure may incorporate the operation of, for one or more points on the first mesh, projecting a ray normal to the mesh surface and calculating the distance before that ray is incident upon the second mesh. The lengths of the resulting line segments may be used to quantify the distance between the meshes. According to some techniques of this disclosure, the distance may be assigned a color based on the magnitude of that distance and that color may be applied to the first mesh, by way of visualization.

Oral care parameters may include one or more values that specify orthodontic procedure parameters, or restoration design parameters (RDP), as described herein. Oral care parameters may define one or more intended aspects of a 3D oral care representation and may be provided to an ML model to promote that ML model to generate output which may be used in the generation of oral care appliances that are suitable for the treatment of a patient. Other types of values include doctor preferences and restoration design preferences, as described herein. Doctor preferences and restoration design preferences may define the typical treatment choices or practices of a particular clinician. Restoration design preferences are subjective to a particular clinician, and so differ from restoration design parameters. In some implementations, doctor preferences or restoration design preferences may be computed by unsupervised means, such as clustering, which may determine the typical values that a clinician uses in patient treatment. Those typical values may be stored in a datastore and recalled to be provided to an automated ML model as default values (e.g., default values which may be modified before execution of the model).

For example, one clinician may prefer one value for a restoration design parameter (RDP), while another clinician may prefer a different value for that RDP, when faced with a similar diagnosis or treatment protocol. One example of such an RDP is dental restoration style. Procedure parameters and/or doctor preferences may, in some implementations, be provided to a setups prediction model for orthodontic treatment, for the purpose of improving the customization of the resulting orthodontic appliance. Restoration design parameters and doctor restoration preferences may in some implementations be used to design tooth geometry for use in the creation of a dental restoration appliance, for the purpose of improving the customization of that appliance. In addition to oral care parameters, doctor preferences, and doctor restoration preferences, some implementations of ML prediction models of this disclosure, in orthodontic treatment, may also take as input a setup (e.g., an arrangement of teeth). In some such implementations, an ML prediction model of this disclosure may take as input a final setup (i.e., final arrangement of teeth), such as in the case of a prediction model trained to generate intermediate stages. For simplicity, these preferences are referred to as doctor restoration preferences, but it is intended to be used in a non-limiting sense. Specifically, it should be appreciated that these preferences may be specified by any treating or otherwise appropriate medical professional and are not intended to be limited to doctor preferences per se (i.e., preferences from someone in possession of an M.D. or equivalent degree).

An oral care professional or clinician, such as a dentist or orthodontist, may specify information about patient treatment in the form of a patient-specific set of procedure parameters. In some instances, an oral care professional may specify a set of general preferences (aka doctor preferences) for use over a broad range of cases, to use as default values in the set of procedure parameters specification process. Oral care parameters may in some implementations be incorporated into the techniques described in this disclosure, such as one or more of GDL Setups, VAE Setups, RL Setups, Setups Comparison, Setups Classification, VAE Mesh Element Labelling, MAE Mesh In-Filling, Validation Using Autoencoders, Imputation of Missing Procedure Parameters Values, Metrics Visualization, or FDG Setups. One or more of these models may take as input one or more procedure parameters vector K and/or one or more doctor preference vectors L. In some implementations, one or more of these models may introduce to one or more of a neural network's hidden layers one or more procedure parameters vector K and/or one or more doctor preferences vectors L. In some implementations, one or more of these models may introduce either or both of K and L to a mathematical calculation, such as a force calculation, for the purpose of improving that calculation and the ultimate customization of the resulting appliance to the patient.

Some implementations of a neural network for predicting a setup (such as GDL Setup, VAE Setup or RL Setup) may incorporate information from an oral care professional (aka doctor). This information may influence the arrangement of teeth in the final setup, bringing the positions and orientations of the teeth into conformance with a specification set by the doctor, within tolerances. In some implementations of the GDL Setup model, oral care parameters may be provided directly to the generator network as a separate input alongside the mesh data. In some implementations of GDL Setups, oral care parameters may be incorporated into the feature vector which is computed for each mesh element before the mesh elements are input to the generator for processing. Some implementations of a VAE Setup model may incorporate oral care parameters into the setups predictions. In some implementations, the procedure parameters K and/or the doctor preference information L may be concatenated with the latent space vector C. A doctor's preferences (e.g., in an orthodontic context) and/or doctor's restoration preferences may be indicated in a treatment form, or they could be based upon characteristics in treatment plans such as final setup characteristics (e.g., amount of bite correction or midline correction in planned final setups), intermediate staging characteristics (e.g., treatment duration, tooth movement protocols, or overcorrection strategies), or outcomes (e.g., number of revisions/refinements).

The restoration treatment of the patient may involve the specification of one or more of the following: restoration guidelines, restoration design parameters, and/or restoration rules for modifying one or more aspects of a patient's dentition. One or more of many possible factors may be considered in designing a 3D restoration, whether from an esthetic standpoint and/or from a technical standpoint. For instance, from an esthetic standpoint, the dental and facial midlines and angulation may provide overall guidance, as does the amount of tooth seen by others when the lips are at rest and/or when smiling. After those criteria are considered, a set of “golden proportions” may also inform the esthetic design of overall tooth sizes. Tooth-to-tooth proportion may be configured to reflect these “golden proportions,” which are: 1.618:1.0:0.618 for the central incisor, lateral incisor and the canine, respectively. In some implementations, a real value may be specified for one or more of the RDP and be received at the input of a dental restoration design prediction model (e.g., a machine learning model to predict the final tooth shape at the completion of restoration design). In some implementations, one or more RDP may be defined which correspond to one or more restoration design metrics (RDM).

Limitations based on tooth position (i.e., malocclusion) and orientation (i.e., rotation and inclination) are balanced against trying to achieve proper symmetry, tooth proportion, and tooth-to-tooth proportion. After these parameters are established, a variety of tooth shapes may be leveraged to match the overall esthetic of the patient's face and smile. For example, tooth shapes may be generally rectangular with squared edges, or they may be generally ovoid with rounded edges. Additionally, tooth-to-tooth proportions may be manipulated to invoke a different overall esthetic. 3D Dental CAD programs often provide libraries of different tooth “styles” to choose from and offer designers the ability to tune the result to best match the esthetic and medical requirements of the doctor and patient. In some examples, symmetry may be observed in that the left side should mirror the right side, and symmetry may thereby be measured.

Tooth length, width, and width-to-length esthetic relationships may be specified for one or more teeth. In one example, the length for a maxillary central incisor may be set to 11 mm, and the width-to-length esthetic relationship may be set to either 70% or 80%. In some examples, the lateral incisors may be between 1.0 mm and 2.5 mm shorter than the central incisors. Canine teeth may, in some instances, be between 0.5 mm and 1.0 mm shorter than the central incisors. Other proportions and measurements are possible for various teeth.

From a technical standpoint, there are other considerations that may be taken into account. For instance, restorations produced from a given material must be of sufficient thickness to have the necessary mechanical strength required for long term use. Additionally, the tooth width and shape must be designed to provide a suitable contact with the adjacent teeth.

The example Style options in the list below are from the LVI standards, from the Las Vegas Institute (LVI) of Advanced Dental Studies. Other style guides are commercially or freely available.

In these and/or other examples, a neural network engine of this disclosure may incorporate as an input one or more of accepted “golden proportion” guidelines for the size of teeth, accepted “ideal” tooth shapes, patient preferences, practitioner preferences, etc.

Restoration design parameters (RDP) may be used to encode aspects of smile design guidelines described herein, such as parameters which pertain to the intended dimensions of a restored tooth. Restoration design parameters are intended as instructions and/or specifications which describe the shape and/or form that one or more teeth should assume after the completion of dental restoration treatment. One or more RDP may be received by a neural network or other machine learning or optimization algorithm for dental restoration design, with the advantage of providing guidance to that optimization algorithm. Some neural networks may be trained for dental restoration design generation, such as some examples of a GAN or an autoencoder. In some instances, a dental restoration design may be used to define the target shapes of one or more teeth for the generation of a dental restoration appliance. In some instances, a dental restoration design may be used to define the target teeth shapes for the generation of one or more veneers.

A partial list of tooth dimensions may include length, width, height, circumference, diameter, diagonal measure, volume-any of which dimensions may be normalized in comparison to another tooth or teeth. In some implementations, one or more restoration design parameters may be defined which pertain to a gap between two or more teeth, and the amount, if any, of the gap which the patient wishes to remain after treatment (e.g., such as when a patient wishes to retain a small gap between the upper central incisors).

Additional restoration design parameters may include the parameters specified in the following table. In the event that one of these parameters contradicts another, the following order may determine precedence (i.e., let the first parameter in the following list be considered authoritative). If a parameter value is not specified, then that parameter may be ignored. In some implementations, default values may be introduced for one or more parameters. Such default values may be determined, for example, through clustering of prior patient cases. A golden proportion guideline may specify one or more numbers pertaining to the widths of adjacent teeth, such as: {1.6, 1, 0.6}.

TABLE 2

| | Parameter Value of |
|---|---|
| Parameter Name | Unit of Measure |
| Golden proportion guideline | {guideline01, guideline02, |
| | guideline03, guideline04, etc.} |
| Tooth width at base (mesial | [millimeters] |
| to distal distance) | |
| Tooth width at incisal edge | [millimeters] |
| (mesial to distal distance) | |
| Tooth height (gingival to | [millimeters] |
| incisal distance) | |
| Width-to-length esthetic | [percentage] |
| relationship | |
| Tooth-to-tooth proportion - | [real number] |
| upper right central incisor width | |
| to upper lateral incisor width | |
| Tooth-to-tooth proportion - | [real number] |
| upper right lateral incisor width | |
| to upper cuspid width | |
| Tooth-to-tooth proportion - | [real number] |
| lower right central incisor width | |
| to lower lateral incisor width | |
| Tooth-to-tooth proportion - | [real number] |
| lower right lateral incisor width | |
| to lower cuspid width | |
| Tooth-to-tooth proportion - | [real number] |
| upper left central incisor width | |
| to upper lateral incisor width | |
| Tooth-to-tooth proportion - | [real number] |
| upper left lateral incisor width | |
| to upper cuspid width | |
| Tooth-to-tooth proportion - | [real number] |
| lower left central incisor width | |
| to lower lateral incisor width | |
| Tooth-to-tooth proportion - | [real number] |
| lower left lateral incisor width | |
| to lower cuspid width | |
| Overall tooth shape | {rectangular or ovoid, squared edges |
| (more than one may apply) | or rounded edges} |
| Amount of tooth display when | [millimeters] |
| the lips are at rest | |
| Dental restoration style | {youth, geriatric, natural, oval, etc.} |
| Style | {dominant, aggressive, focused, enhanced, |
| | functional, natural, Hollywood, mature, |
| | vigorous, youthful, oval, softened, or other |
| | examples from the LVI style guide, etc.} |
| Tooth morphology - shape style | [triangular, oval and rectangular, etc.] |
| guide | |
| Tooth morphology - angle lines | [lines_style01, lines_style02, lines_style03, |
| | etc.] |
| Tooth morphology - incisal angles | [angles_style01, angles_style02, angles_style03, |
| and embrasures | etc.] |
| Tooth morphology - buccal contour | [contour_style01, contour_style02, |
| | contour_style03, etc.] |
| Tooth morphology - Mamelon grooves | [mamelon_style01, mamelon_style02, |
| | mamelon_style03, etc.] |
| Tooth morphology - perikymata | [perikymata_style01, perikymata_style02, |
| | perikymata_style03, etc.] |

Tooth-to-tooth proportions may also be defined between other pairs of teeth as well. Proportions may be made relative to tooth widths, heights, diagonals, etc. Angle lines, incisal angles and buccal contours may describe primary aspects of tooth macro shape. Mamelon grooves may be vertical macro textures on the front of a tooth, and sometimes may take a V-shape. Striations or perikymata may be a horizontal micro texture on the teeth. Symmetry may be generally desired. There may be differences between male and female patients.

Parameters may be defined to encode doctor restoration design preferences (DRDP), as pertains to various use case scenarios. These use case scenarios, may reflect information about the treatment preferences of one or more doctors, and directly affect the characteristics of one or more teeth in a dental restoration design or a veneer. In addition, DRDP may describe preferred or habitually involved values or ranges of values of RDP for a doctor or other treating medical professional. In some instances, such value or ranges of values may be derived from historical patient cases that were treated by that doctor or medical professional. In some instances, a DRDP may be defined which is derived from a RDP (e.g., such as Width-to-length esthetic relationship) or from a RDM. Non-limiting examples of RDP are described in Table 2.

Machine learning models, such as those described herein, may be trained to generated designs for tooth crowns or tooth roots (or both). A dental restoration design may describe a tooth shape which is intended at the end of dental restoration. A neural network (such as a generative neural network) may be trained to generate a dental restoration design which is to be used to produce either a veneer (e.g., a zirconia veneer) or a dental restoration appliance. Such a model make take as input data from past patient cases, including pre-restoration tooth meshes and corresponding ground truth examples of completed restorations (e.g., tooth meshes with restored shapes and/or structures). Such a model may be trained, at least in part through the calculation of a loss function, which may quantify the difference between a generated crown restoration design and a ground truth crown restoration design. The resulting loss may be used to update the weights of the generative neural network model (e.g., a transformer), thereby training the model (at least in part). A reconstruction loss may be computed to compare a predicted tooth mesh to a ground truth tooth mesh, or to compare pre-restoration tooth mesh to a completed restoration design tooth mesh. Reconstruction loss may be computed as the sum of pairwise distances between corresponding mesh elements and may be computed to quantify the difference between two tooth crown designs.

Other losses disclosed herein may also be in the training. A transformer may improve data precision and may be especially suited to the generation of a restoration design for a crown, due to the large number of mesh elements such a mesh may contain. Transformers have been shown to be adept at processing long sequences of data, and other large datasets. A veneer may be created using the generated restoration design. Such as veneer may be 3D printed.

Machine learning models, such as those described herein, may be trained to generate components for use in creating a dental restoration appliance. Such a dental restoration appliance may be used to shape dental composite in the patient's mouth while that composite is cured (e.g., using a curing light), to ultimately produce veneers on one or more of the patient's teeth. The 3M® FiltekT^{M} Matrix is an example of such a product. In some instances, a machine learning model for generating an appliance component may take inputs which are usable to customize the shape and/or structure of the appliance component, including inputs such as oral care parameters. One or more oral care parameters may, in some instances, be defined based on oral care metrics. An oral care metric (e.g., an orthodontic metric or a restoration design metric) may describe physical and/or spatial relationships between two or more teeth or may describe physical and/or dimensional characteristics of an individual tooth. An oral care parameter may be defined which is intended to provide a machine learning model with guidance on generating a 3D oral care representation with a particular physical characteristic (e.g., pertaining to shape and/or structure). For example, physical characteristics may be measured with an oral care metric to which that oral care parameter corresponds. Such oral care parameters may be defined to customize the generation of mold parting surfaces, gingival trim meshes or other generated appliance components, to tailor those appliance components to the patient's dental anatomy.

In some implementations, the 3D representation generation techniques described herein (e.g., transformer-based techniques) may be trained to generate the custom appliance components by determining the characteristics of the custom appliance components, such as a size, shape, position, and/or orientation of the custom appliance components. Examples of custom appliance components include a mold parting surface, a gingival trim surface, a shell, a facial ribbon, a lingual shelf (also referred to as a “stiffening rib”), a door, a window an incisal ridge, a case frame sparing, or a diastema matrix wrapping, among others.

A mold parting surface refers to a 3D mesh that bisects two sides of one or more teeth (e.g., by separating the facial side of one or more teeth from the lingual side of the one or more teeth). A gingival trim surface refers to a 3D mesh that trims an encompassing shell along the gingival margin. A shell refers to a body of nominal thickness. In some examples, an inner surface of the shell matches the surface of the dental arch and an outer surface of the shell is a nominal offset of the inner surface.

The facial ribbon refers to a stiffening rib of nominal thickness that is offset facially from the shell. A window refers to an aperture that provides access to the tooth surface so that dental composite can be placed on the tooth. A door refers to a structure that covers the window. An incisal ridge provides reinforcement at the incisal edge of a dental restoration appliance and may be derived from the archform. The case frame sparing refers to connective material that couples parts of a dental restoration appliance (e.g., the lingual portion of a dental restoration appliance, the facial portion of a dental restoration appliance, and subcomponents thereof) to the manufacturing case frame. In this way, the case frame sparing may tie the parts of a dental restoration appliance to the case frame during manufacturing, protect the various parts from damage or loss, and/or reduce the risk of mixing up parts.

Additional 3D oral care representations which may be generated by a transformer (such as transformers trained as described herein) including tooth interproximal surfaces and tooth roots.

In some instances, the transformers described herein may be trained to perform 3D mesh element labeling (e.g., labeling vertices, edges, faces, voxels, or points) in 3D oral care representations. Those labeled mesh elements may be used for mesh cleanup or mesh segmentation. In the case of mesh cleanup, the labeled aspects of a scanned tooth mesh may be used for appliance erasure (removal+replacement) or be used to modify (e.g., by smoothing) one or more aspects of the tooth to remove aspects of attached hardware (or other aspects of the mesh which may be unwanted for certain processing and appliance creation—such as extraneous material). Mesh element features, such as those described herein, may be computed for one or more mesh elements in a 3D oral care representation. A vector of such mesh element features may be computed for each mesh element and then be received by a transformer which has been trained to label mesh elements in a 3D oral care representation for the purpose of either mesh segmentation or mesh cleanup. Such mesh element features may confer valuable information about the shape and/or structure of the input mesh to the labeling transformer.

Some implementations of the transformer-based mesh cleanup techniques described herein may train the transformers to remove (or modify) generic triangle mesh defects, such as: degenerate triangle with zero surface area; redundant triangle that covers the same surface area as another triangle; non-manifold edge with more than two adjacent triangles, also referred to as a “fin”; non-manifold vertex with more than one adjacent sequence of connected triangles (triangle fans); intersecting triangles—where two triangles penetrate each other; spikes—sharp features composed of multiple triangles, often conical, caused by one or more vertices being displaced from the actual surface; folds—sharp features composed of multiple triangles, often Z-shaped with a small undercut area, caused by one or more vertices being displaced from the actual surface; islands/small components—disconnected objects in a scan which should only contain a single object (e.g., typically the smaller objects are deleted); small holes in the mesh surface, either from the original scan or from deletions due to the previous defects (e.g., the hole may be removed by filling the hold, for example by adding one or more mesh elements); rough boundary—a smooth boundary is desirable for extending the gingiva surface, creating a model base.

Some implementations of the transformer-based mesh cleanup techniques described herein may train the transformer models to remove (or modify) aspects of meshes which are unwanted under certain circumstances and/or domain-specific defects, such as: extraneous material—portions of the intraoral scan outside the anatomical area of interest, e.g., non-tooth surfaces that are not within some distance of tooth surfaces, or scan artifacts that do not represent actual anatomy; divots—concave depressions in surfaces (e.g., may be scan artifacts, which should be fixed, or anatomical features, which are generally left intact); undercuts—sides of a tooth of lower radius than the crown, such that physical impressions or aligners may become difficult to remove or emplace. Undercuts may be a natural feature or due to damage such as an abfraction. Abfractions are associated with the erosion of a tooth near the gumline, causing or exacerbating an undercut. Appliances that the transformer-based models of this disclosure may process include orthodontic hardware such as attachments, brackets, wires, buttons, lingual bars, Carriere appliances, or the like, and may be present in intraoral scans. Digital removal and replacement with synthetic tooth/gingiva surfaces may, in some circumstances, be beneficial if performed before appliance creation steps proceed.

Neural networks of this disclosure trained for the placement and/or generation of oral care appliance components (e.g., such as dental restoration appliance {DRA}components) may, in some implementations, operate on a patient's post-restoration dentition. In other implementations, such a neural network may operate on a pre-restoration dentition of the patient.

Aspects of mesh element features are described below. In some implementations, either or both of a neural network to generate a first component for a dental restoration appliance (DRA) and/or a neural network to place a second component for a DRA may input one or more mesh element features, with the advantage of improving the neural network's processing precision with respect to the inputted 3D representation(s) (i.e., such as teeth, gums, hardware, and/or third components). Mesh element features are described elsewhere in this disclosure. Mesh element features may also be used in the placement of brackets and/or attachments. Mesh element features may also be used in the generation of 3D representations of veneers and/or crowns.

Aspects of this disclosure use representation learning (RL) for DRA component placement. The RL-based techniques of this disclosure may use any of a VAE, Capsule Autoencoder, or U-Net to create the representations of the teeth and library component. In some implementations, the RL-based techniques of this disclosure may use a downstream encoder, transformer, or multi-layer perceptron (MLP) network to generate the transformation to place the DRA component (or bracket or attachment) relative to one or more teeth.

Various aspects of this disclosure may employ the use of generative models as well. In some implementations, systems of this disclosure may use an autoencoder to generate a component for a DRA. The inputted 3D representations of the teeth may be encoded into a latent form A (either latent vector or latent capsule), and may apply modifications to A. These modifications may be made according to prior experiments which mapped out the latent space. In some implementations, the reconstructed output of such a model may be a new DRA component. In some implementations, where an existing DRA component was provided to the autoencoder alongside the teeth, the reconstructed output may comprise a modified DRA component (e.g., with improved shape and fit relative to the teeth). Generative models may also be used in the generation of 3D representations of veneers and/or crowns, among other appliances.

Some of the neural networks of this disclosure may take, as inputs, restoration design metrics (RDM) may be taken as inputs to a neural network to place a DRA component. Likewise, RDM may be taken as inputs to a neural network to generate a DRA component. Neural networks of this disclosure that use RDM data as input is that the neural network may obtain, by way of the RDM, knowledge about the geometry and/or structure of the patient's dentition.

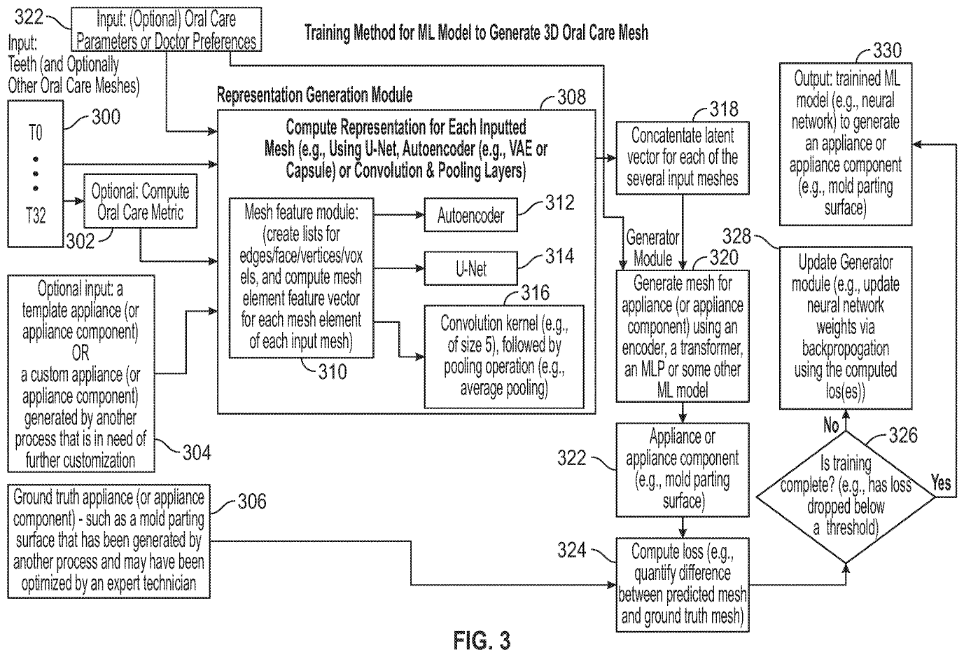

FIG. 3 describes a method to train a machine learning model to modify or generate a predicted 3D oral care representation (e.g., training a neural network to generate or modify an oral care appliance component, a trimline, an archform, a tooth crown restoration design, or the like) in accordance with aspects of this disclosure. Oral care meshes 300 (e.g., representing the patient's teeth) may be received at the input. Optional oral care metrics may be computed (310), including orthodontic metrics which may describe physical relationships between teeth (e.g., pertaining to positions and orientations of teeth relative to other teeth or to the gums) and/or restoration design metrics which may describe physical aspects within a tooth. The tooth meshes 300, any optional oral care metrics 302 computed on those teeth, and other optional inputs may be provided to the representation generation module 308. Optional oral care parameters or doctor preferences 332 may be provided to representation generation module 308 or to generator module 320, to customize the outputs of those modules. Other optional inputs 304 may include a template oral care mesh (e.g., an appliance or appliance component, such as a parting surface) or a customized oral care mesh (e.g., such as a mold parting surface generated according to the techniques of either WO2021240290A1 or WO2020240351A1) which may require further customization. Ground truth 3D oral care representations 306 may be received at the input to the method, and used in loss calculation (e.g., according to the loss calculation techniques described herein). In the case of appliance component generation, the inputs may comprise the patient's teeth 300, and optional template appliance component 304 (which may help influence the generator 320 in generating a predicted appliance component) and a ground truth appliance component. Such an appliance component may comprise any of a mold parting surface, gingival trim surface, a shell, a facial ribbon, a lingual shelf (also referred to as a “stiffening rib”), a door, a window, an incisal ridge, a case frame sparing, a diastema matrix wrapping, among others. Optional mesh element features may be computed for each mesh element in 300, 302 and/or 304, after which representations may be generated for these oral care meshes. A representation may be generated using an autoencoder 312, which yields a latent vector or latent capsule. A representation may be generated using a U-Net 314, which yields an embedding vector. A representation may also be generated using a pyramid encoder-decoder or by using an MLP comprising convolution and pooling layers 316 (e.g., with convolution kernel size 5 and average pooling). Other representations are possible in accordance with aspects of this disclosure. The representations may be concatenated (318) and received by the generator module 320, which may generate one or more predicted 3D oral care representations (e.g., using any of the following used in combination with mesh element feature vectors: a transformer, an autoencoder, PolyGen or a neural network trained off of PolyGen via transfer learning). Loss may be computed (324) between the generated oral care mesh 322 and the corresponding ground truth oral care mesh 306. Training may be considered complete after loss falls below a threshold (326). In the course of training the generator, loss may be fed back to update the weights of the generator (328), for example, using backpropagation. The output of the method 330 may include a trained machine learning model (e.g., a neural network) for generating predicted oral care meshes.

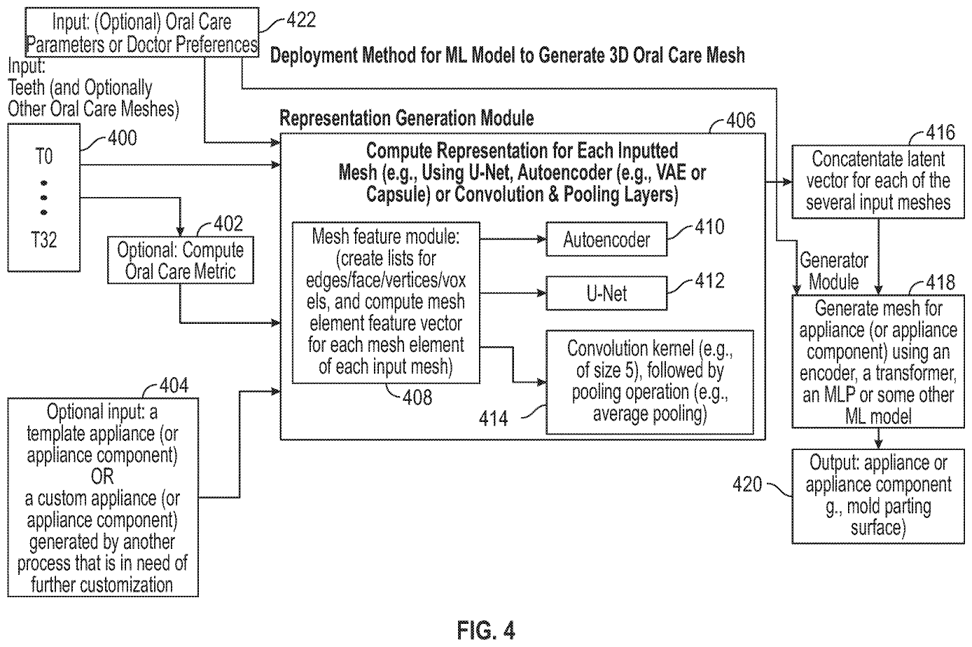

FIG. 4 describes a method for a deployed machine learning model to generate a predicted 3D oral care representation (e.g., to generate an oral care appliance component). Oral care meshes 400 (e.g., the patient's teeth) may be received at the input. Optional oral care metrics may be computed (402). The tooth meshes 400, any optional oral care metrics 402 computed on those teeth, and other optional inputs may be received by the representation generation module 406. Other optional inputs 404 may include a template oral care mesh or a customized oral care mesh which may require further customization. Optional oral care parameters or doctor preferences 422 may be provided to representation generation module 406 or generator module 418, to customize the outputs of those modules. Optional mesh element features may be computed (408) for each mesh element in 400, 402 and/or 404, after which representations may be generated for these oral care meshes. A representation may be generated using an autoencoder 410, which yields a latent vector or a latent capsule. A representation may be generated using a U-Net 412, which yields an embedding vector. A representation may also be generated using a pyramid encoder-decoder or by using an MLP comprising convolution and pooling layers 414 (e.g., with convolution kernel size 5 and average pooling). Other representations are possible in accordance with aspects of this disclosure. The representations may be concatenated (416) and received by the generator module 418, which may generate one or more predicted 3D oral care representations (e.g., using an autoregressive generative neural network model, such as PolyGen or a neural network trained off of PolyGen via transfer learning). The output 420 may include one or more predicted 3D oral care representations (e.g., a mesh describing a mold parting surface).

In some implementations, the method in FIG. 3 may be trained to modify one or more inputted 3D oral care representations, such as inputted appliance components, inputted tooth designs (e.g., pre-restoration or in-progress restoration designs), trimlines, archforms, or other types of 3D oral care representations. In some instances, a transformer, such as a generative autoregressive transformer (e.g., PolyGen) may be trained to modify the 3D oral care representations. Such modification may entail operations such as: adding or imputing one or more mesh elements, removing one or more mesh elements, point cloud completion, transforming one or more mesh elements (e.g., modifying the position and/or orientation of one or more mesh elements), and/or the like. One or more of the neural networks of FIG. 3 (such as a transformer from the generator modules) may in some implementations be trained, at least in part, by transfer learning. One or more of the neural networks which is trained in FIG. 3 may subsequently be used to train, at least in part, another neural network (such as a neural network for some aspect of digital oral care automation) according to transfer learning paradigms. Mesh element feature vectors may be computed for one or more mesh elements of one or more inputs to FIGS. 3 and 4, which may enable improved understanding of those input meshes or point clouds.

FIG. 3 shows a training method, and FIG. 4 shows a deployment method. These methods may pertain to the use of neural networks to generate oral care appliances or oral care appliance components. In some optional scenarios, the neural networks of this disclosure may further customize or improve upon existing appliances or appliance components, in which case an appliance or appliance component which has an initial configuration may be received as input data to the neural network. In some implementations, the training method in FIG. 3 may be executed to generate a component for a dental restoration appliance (e.g., such as a mold parting surface, gingival trim surface, a shell, a facial ribbon, a lingual shelf (also referred to as a “stiffening rib”), a door, a window, an incisal ridge, a case frame sparing, a diastema matrix wrapping, among others). A spline refers to a curve that passes through a plurality of points or vertices, such as a piecewise polynomial parametric curve. A mold parting surface refers to a 3D mesh that bisects two sides of one or more teeth (e.g., separates the facial side of one or more teeth from the lingual side of the one or more teeth). A gingival trim surface refers to a 3D mesh that trims an encompassing shell along the gingival margin. A shell refers to a body of nominal thickness. In some examples, an inner surface of the shell matches the surface of the dental arch and an outer surface of the shell is a nominal offset of the inner surface. The facial ribbon refers to a stiffening rib of nominal thickness that is offset facially from the shell. A window refers to an aperture that provides access to the tooth surface so that dental composite can be placed on the tooth. A door refers to a structure that covers the window. An incisal ridge provides reinforcement at the incisal edge of dental appliance and may be derived from the archform. The case frame sparing refers to connective material that couples parts of a dental appliance (e.g., the lingual portion of a dental appliance, the facial portion of a dental appliance, and subcomponents thereof) to the manufacturing case frame. In this way, the case frame sparing may tie the parts of a dental appliance to the case frame during manufacturing, protect the various parts from damage or loss, and/or reduce the risk of mixing-up parts. These appliance components and others are described in PCT patent applications WO2020240351A1 and WO2021240290A1, both of which are incorporated herein by reference in their entirety.

3D representations (such as 3D meshes) of the patient's teeth may be received at the input of the training method in FIG. 3, along with an associated ground truth appliance or appliance component (e.g., such as a ground truth mold parting surface) that may have been generated by an automation model (e.g., such as the techniques of WO2020240351A1 or WO2021240290A1) and may be modified or revised by an expert technician or other healthcare practitioner or clinician. In some implementations, the training method of FIG. 3 may be enhanced by the calculation of one or more oral care metrics on the received teeth. Oral care metrics include orthodontic metrics (OM), which may describe relationships between two or more teeth, and dental restoration metrics (DRM), which may describe aspects of the shape and/or structure of an individual tooth (and in some instances may describe relationships between two or more teeth). These oral care metrics (which are described elsewhere) may help the representation module to create representations of the teeth. A representation of a tooth may reduce the size or quantity of data required to describe the tooth's shape and/or structure, while retaining much of the information about that tooth's shape and/or structure, thereby providing a computing resource usage reduction-based technical improvement of this disclosure. The representation of the tooth may be more easily consumed by a machine learning model (such as the generator module) in this reduced-size and compact form.

Neural networks of this disclosure may generate a representation of a tooth or other oral care mesh, such as a received appliance or appliance component. The tooth mesh may be reconfigured into one or more lists of mesh elements (e.g., vertices, faces, edges, or voxels). For each mesh element, an optional mesh element feature vector may be computed, according to the mesh element feature descriptions provided elsewhere in this disclosure. This mesh element features may help a neural network in the encoding of a tooth mesh into a reduced-size representation. An autoencoder, such as a variational autoencoder or capsule autoencoder, may be trained to reconstruct the oral care mesh (e.g., such as a tooth or an appliance component) in accordance with aspects of this disclosure. A trained reconstruction autoencoder may use the 3D encoder stage to encode the mesh into a latent vector (or latent capsule). This latent vector (or latent capsule) may be used as the representation of an oral care mesh.

In accordance with various techniques of this disclosure, alternatives to autoencoders include U-Net neural network structures. A U-Net may encode an oral care mesh into an embedding vector, which may then be used as the tooth representation. In some implementations, one or more layers comprising convolution kernels and pooling operations may be trained to perform the encoding task. For example, a convolution kernel of size five (5) may be combined with an average pooling operation to effect the encoding of an oral care mesh into a representation that is suitable to be received by the generator module.

The generator module may receive representations of the teeth, appliance components, and/or any other oral care meshes. These representations may, in some implementations, be concatenated before being received by the generator module. The generator module may comprise a neural network or some other machine learning model. In some implementations, a multilayer perceptron (MLP) may be trained to receive the concatenated oral care representations and output a mesh corresponding to an appliance or appliance component. The output layers of the encoder may be designed to output an appliance component, such as a mold parting surface. A mold parting surface may be described using a 3D mesh and may comprise a significant quantity of mesh elements. The output layers of the generator module may accommodate an output of dozens or even hundreds of generated mesh elements, a circumstance for which a transformer-based model may be especially well suited. In some implementations, a transformer may be used in place of the MLP. Other implementations may replace the MLP or transformer with a 3D encoder. In some implementations, the generator module may include a transformer configured to generate or modify at least some aspect of a received representation (e.g., such as a reformatted representation that is output by the representation generation module). That transformer may, in some instances be followed by one or more other neural networks (e.g., a set of fully connected layers) which may further reformat or rearrange the transformer outputs, such as to rearrange the outputs into one or more 3D meshes or point clouds, which may then be outputted by the generator module.

The output of the generator module may comprise a mesh, such as a mold parting surface (or other appliance or appliance component or other type of oral care representation), which may be received by a loss calculation module. The loss calculation module may also receive a ground truth mold parting surface (or other appliance or appliance component) and proceed to quantify the difference between the predicted and ground truth meshes. This loss may be used to train, at least in part, the generator module. The loss may be used to modify the weights of the generator module (in the case that the generator module is a neural network) via a backpropagation algorithm. The training method is considered complete after loss drops below a designated threshold (perhaps as shown over a number of successive iteration). The generator may be trained using at least one of KL divergence loss and reconstruction loss (e.g., which may involve a vertex-to-vertex distance computation). Additional losses which may be used include normalized L1 and L2 distances, chamfer loss and MSE loss (e.g., normalized MSE loss). In some instances, the training method may be considered complete once the generator's accuracy rises above a designated threshold. Accuracy may be computed using an ADD score. The ADD score may measure the percentage of patient cases which are closer than a threshold distance from each other (e.g., measured using L2 distance, or another of the distance measurement techniques described herein). The threshold distance may, in some implementations, be determined adaptively based on the size of the ground truth mesh. After the completion of training (e.g., upon achieving a stable state or convergence), the trained model may be outputted.

The generator module (GM) may comprise an autoregressive machine learning model which is trained to predict custom 3D oral care representations for a new patient based on examples of 3D oral care representations from past patients. In some implementations, a U-Net may be trained to autoregressively generate oral care meshes. In other instances, a transformer (e.g., PolyGen) may be trained to autoregressively generate oral care meshes. Such a transformer may be trained to model a distribution over examples of a particular type of oral care mesh, examples of which are described elsewhere in this disclosure. Such a transformer may comprise one or more neural networks, each of which is trained to model a distribution over a particular mesh element. For example, a first neural network may be trained to model an unconditional distribution over mesh vertices, and a second neural network model may be trained to conditionally model a distribution over mesh faces (or alternatively, mesh edges, or in case of sparse processing, voxels). First the vertices of the predicted oral care mesh are estimated, and then the faces (or alternatively, edges) which connect those vertices are estimated based on the received set of estimated vertices. The first model (which estimates vertices) may receive tooth meshes as inputs, with meshes for hardware attached to the teeth and/or meshes for appliances or appliance components.

Such appliances or appliance components may represent templates which serve as the starting point for oral care mesh generation. In other instances, such appliances or appliance components may be previously customized and are subject to further modifications and/or further improvements from the transformer. The first model (for estimating vertices) may comprise a transformer decoder, such as is described by (Vaswani, Ashish & Shazeer, Noam & Parmar, Niki & Uszkoreit, Jakob & Jones, Llion & Gomez, Aidan & Kaiser, Lukasz & Polosukhin, Illia, “Attention is all you need”, 2017.). The second model may model a conditional distribution over faces and may assemble the vertices estimated by the first model into true 3D meshes.

The GM may be trained to generate different kinds of oral care meshes, such as appliance components (e.g., the model parting surface), a clear tray aligner trimline (e.g., such as those implemented using a polyline or a mesh), or an archform (e.g., such as those implemented using a mesh or a set of control points with an associated spline curve through those control points), a tooth restoration design (e.g., a target tooth shape and/or structure which is intended at the completion of dental restoration treatment, which may be used in the creation of a dental restoration appliance), a crown design, or a veneer design (e.g., a zirconia veneer). To generate a given kind of 3D oral care representation, numerous examples of training data of that (or a related) kind of 3D oral care representation may be introduced into the training and validation datasets. A holdout test set may be used to evaluate the accuracy of predicted representations.

In terms of archform prediction, a generative machine learning model, such as a neural network, may be defined to predict an archform. Archforms are defined elsewhere in this disclosure. In some implementations, an archform may be defined, at least in part, by one or more of a polyline and a set of control points (where such control points describe one or more splines or other curves which may be fitted to the control points). In some implementations, an ML model may be trained, at least in part, through the use of a loss function which quantifies the difference between a predicted archform and a reference archform (such as a ground truth archform which was previously configured by an expert or by an optimization algorithm). Such an ML model for archform prediction may be trained, at least in part, on data from past patient cases, where a patient case contains at least one of a set of segmented tooth meshes for the patient, a mal transform for one or more teeth (e.g., for each tooth), a setup transform for one or more teeth (e.g., for each tooth), or a ground truth archform (which may in some instances exhibit ideal archform characteristics). A predicted archform may be used in patient treatment, such as to be provided to the neural networks of the present disclosure. A neural network for predicting an archform may be based, at least in part, on at least one aspect of at least one of the neural networks or neural network features disclosed elsewhere in this disclosure.

An archform may describe the contours of a dental arch and may, in some instances, describe a smoothed, averaged, or idealized arrangement of teeth in an arch. An archform may, in some instances, align (at least approximately) with a target arrangement of teeth for use in orthodontic treatment, and may in some instances be received as an input to a setups prediction machine learning model, such as a setups prediction neural network for final setups or intermediate staging prediction. An archform may, in some instances, line up with at least one of the incisal edges of the teeth, the gingival margins of the arch, or one or more coordinate systems of one or more teeth.

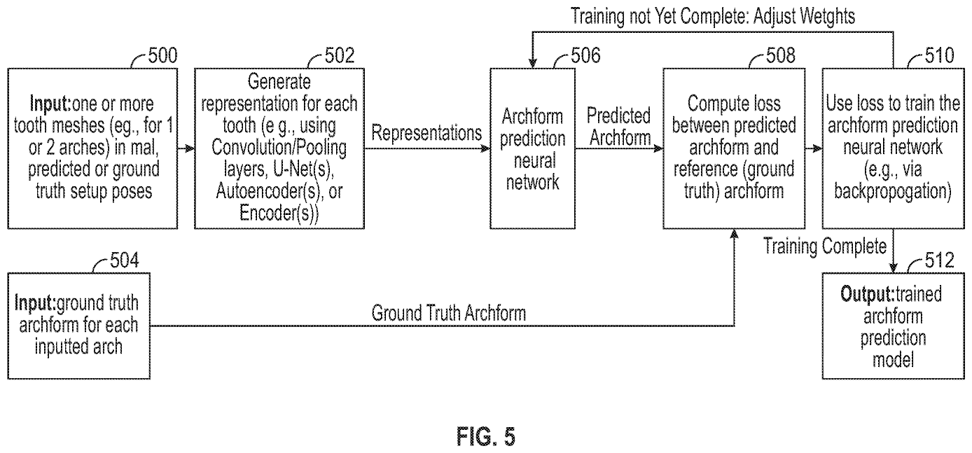

Some of the systems of this disclosure use RL models for archform prediction functionalities. In some implementations, an archform prediction machine learning model (e.g., comprising one or more neural networks) is trained using RL. A representation learning model may comprise a first module, which may be trained to generate a representation of the received 3D oral care representations (e.g., teeth and/or gums), and a second module, which may be trained to receive those representations and generate one or more archforms. FIG. 5 shows an example method for such an implementation. That is, FIG. 5 illustrates an implementation of an archform prediction machine learning model of this disclosure. The tooth meshes of the patient's arches 500 may be provided to a representation generation module 502, which may provide latent representations of the patient's teeth to the archform prediction ML module 506 (e.g., a transformer decoder followed by an MLP, or other structures described herein). The archform prediction ML module 506 may generate one or more predicted archforms, which may be provided to a loss calculation module 508. Loss may be computed which compares the predicted archform to a corresponding ground truth archform 504. The computed loss may be used to train (510) the archform prediction ML module 506. The trained model 512 is outputted.

In some instances, that the generator module (e.g., which may contain at least a transformer, such as a generative autoregressive transformer) of FIG. 3 may be trained to generate a new tooth restoration design or be trained to modify the shape and/or structure of an existing tooth restoration design. Either or both of the representation generation module and/or the generator module may take as inputs oral care parameters, such as restoration design parameters (RDP) and/or doctor restoration design preferences (DRDP). Such oral care parameters may enable the generator module to incorporate clinical instructions from a doctor/dentist/healthcare practitioner, to improve the customization of the resulting restoration design. Either or both of the representation generation module and/or the generator module may take as inputs one or more tooth meshes (crowns and/or roots) from past patient cohort cases.

One or more mesh element features may be computed for one or more mesh elements of one or more of the teeth. Such mesh element features may improve the ability of either or both of the representation and/or generator modules to understand the shape and/or structure of the inputted meshes (e.g., pre-restoration tooth meshes received at the input). A generative autoregressive transformer may be trained to generate various aspects of the shape and/or structure of tooth anatomy based on the distributions of those aspects of tooth anatomy which are found in the training dataset of cohort patient cases. In some instances, a transformer may be trained to impart aspects of the following five levels of tooth design to a generated restoration design (e.g., crown or root). The advantage provided by this technique is to generate a tooth restoration design which reflects one or more of the following physical characteristics: 0—tooth silhouette, e.g., as projected onto a plane in front of the face; 1—main tooth shape (primary anatomy); 2—surface vertical and horizontal macro textures or striations or mamelon grooves (secondary anatomy); 3—surface horizontal micro texture, e.g., perikymata (tertiary anatomy); 4—volumetric representation of the tooth's interior structure (dentine, enamel, etc.).

Transformers may, in some implementations, enable large model capacity and/or enable an attention mechanism (e.g., the capability to focus resources on and respond to certain inputs). Transformers may consume large training datasets and have the advantage in that transformers may continue to grow in model capacity as the training dataset size increases. In contrast, various prior neural networks models may plateau in model capacity as training dataset size grows.

Convolution-based neural networks may, in some implementations, enable fast model convergence during training and improved model generalization. In some implementations for the generation or modification of 3D oral care representations, transformers may be combined with convolution-based neural networks, such as by vertically stacking convolution layers and attention layers. Such stacking may improve efficiency, model capacity and/or model generalization. CoAtNet is an example of a network architecture which combines convolutional and attention-based elements and may be applied to the processing of oral care data. In some instances, a network for the modification or generation of 3D oral care representations may be trained, at least in part, from CoAtNet (or another model that combines convolution and self-attention/transformers) using transfer learning.

Table 3 describes the input data and generated data for several non-limiting examples of the generative techniques described herein. Encoder-decoder structures such as autoencoders or transformers may be trained to generate (or modify) point clouds as described herein. In some implementations, such models may be trained for the generation (or modification) of the input data in Table 3, yielding the generated data in Table 3.

Techniques of this disclosure may be trained to generate (or modify) point clouds (e.g., where a point may be described as a 1D vector—such as (x, y, z)), polylines (points connected in order by edges), meshes (points connected via edges to form faces), splines (which may be computed through a set of generated control points), sparse voxelized representations (which may be described as a set of points corresponding to the centroid of each voxel or to some other landmark of the voxel—such as the boundary of the voxel), a transform (which may take the form of one or more 1D vectors or one or more 2D matrices—such as a 4×4 matrix) or the like. In some implementations, a voxelized representation may be computed from a 3D point cloud or a 3D mesh. In some implementations, a 3D point cloud may be computed from a voxelized representation. In some implementations, a 3D mesh may be computed from a 3D point cloud.

TABLE 3

| Digital oral | | |
|---|---|---|
| care technique | Input data | Generated data |
| Segmentation | 3D representation of patient's | One or more mesh element labels for |
| | pre-segmentation dentition (e.g., | one or more aspects of the patient's |
| | a mesh including teeth and gums) | dentition. A label may indicate which |
| | | mesh elements should be included in |
| | | which segmented portion of the |
| | | patient's dentition. |
| Coordinate | One or more (segmented) teeth | Coordinate system for one or more |
| system | | teeth (or an arch). May comprise |
| | | one or more transforms. |
| Mesh cleanup | 3D representation of patient's | One or more mesh element labels for |
| | dentition (e.g., a mesh including | one or more aspects of the patient's |
| | teeth and gums) | dentition. A label may flag a mesh |
| | | element for removal or modification. |
| Restoration | 3D representation of patient's | 3D representation of patient's |
| design | pre-restoration tooth | intended post-restoration tooth |
| generation | | |
| CTA trimline | 3D representation of patient's | 3D representation of trimline (e.g., |
| | dentition (e.g., a mesh including | 3D mesh or 3D polyline) |
| | teeth and gums) | |
| CTA setups | Two or more tooth meshes and/or | Transforms for one or more teeth |
| (for final setups | tooth transforms. Tooth meshes | |
| or intermediate | may be in their maloccluded poses. | |
| staging) | Transforms may correspond to | |
| | maloccluded poses. | |
| Hardware (e.g., | One or more (segmented) teeth | Transform for placement of hardware |
| bracket/ | | relative to the one or more teeth |
| attachment) | | |
| placement | | |
| Archform | 3D representation of patient's | 3D polyline or a 3D mesh or surface, |
| generation | dentition (e.g., a mesh including | that describes the contours or layout |
| | teeth and gums). May comprise | of an arch of teeth |
| | one or more segmented teeth. | |
| Generated oral | 3D representation of patient's | One or more oral care appliance |
| care appliance | dentition (e.g., a mesh including | components with shape and/or structure |
| component | teeth and gums). May comprise | that is customized to aspects of the |
| (e.g., for | one or more segmented teeth. | patient's dentition |
| dental | | |
| restoration) | | |
| Placed oral | 3D representation of patient's | One or more transforms which place a |
| care appliance | dentition (e.g., a mesh including | library component relative to aspects |
| component | teeth and gums). May comprise | of the patient's dentition |
| (e.g., for | one or more segmented teeth. | |
| dental | | |
| restoration) | | |

The first module may be trained to generate 3D representations for the one or more teeth which are suitable to be consumed by the second module, where the second module is trained to output one or more predicted archforms. In some implementations, one or more layers comprising convolution kernels (e.g., with kernel size 5 or some other size) and pooling operations (e.g., average pooling, max pooling or some other pooling method) may be trained to create representations for one or more teeth in the first module. In some implementations, one or more U-Nets may be trained to generate representations for one or more teeth in the first module. In some implementations, one or more autoencoders may be trained to create representations for one or more teeth (e.g., where the 3D encoder of the autoencoder is trained to encode one or more tooth 3D representations into one or more latent representations, such as latent vectors or latent capsules, where such a latent representation may be reconstructed via the autoencoder's 3D decoder into a facsimile of the input tooth mesh or meshes) in the first module. In some implementations, one or more 3D encoder structures may be trained to create representations for the one or more teeth in the first module. Other techniques of encoding representations are also possible in accordance with the aspects of this disclosure.

The representations of the one or more teeth may be provided to the second module, such as an encoder structure, a multilayer perceptron (MLP), a transformer, an autoencoder (e.g., variational autoencoder or capsule autoencoder), which has been trained to output one or more archforms. In some implementations, an archform may comprise n control points (e.g., n==6), such as those embodied by a 3×6 array (containing the XYZ coordinates for the 6 control points). The model may fit a spline to the n control points, to enhance the description of the archform. In some implementations, an archform may comprise a polyline, a 3D mesh, or a 3D surface. The second module may be trained, at least in part, through the calculation of one or more loss values, such L1 loss, L2 loss, MSE loss, reconstruction loss or one or more of the other loss calculation methods found elsewhere in this disclosure. Such a loss function may quantify the difference between one or more generated representations of archforms and or more reference representation of archforms (e.g., ground truth archforms which are known to be of good function).

Either or both of the first and second modules may, in some implementations, take optional inputs, including one or more of tooth position and/or orientation information O, orthodontic procedure parameters K, orthodontic doctor preferences L, tooth type information, orthodontic metrics S, IPR information U, labels for one or more teeth pertaining to medical condition or medical diagnosis information, etc. The advantage of incorporating one or more of the above-listed inputs is to improve the customization and or function or the archform and improve the suitability of the archfom for use in creating an oral care appliance, such as clear tray aligners or indirect boding trays for orthodontic treatment, thereby yielding a technical improvement in terms of data precision.

Some implementations of a setups prediction neural network may take an archform as input, with the advantage of improving the customization and/or function of the resulting predicted setups (e.g., final setups or intermediate stages). Other setups prediction methods may also benefit from the use of archforms, such as other ML-based setups prediction methods, or non-ML based setups prediction methods. Some implementations of landmark-based setups may benefit from the use of archforms, in the prediction of setups.

An archform may, in some instances, line up with at least one of the incisal edges of the teeth, the gingival margins of the arch, or one or more coordinate systems of one or more teeth. An archform may, in some instances, describe (at least approximately) a target arrangement of teeth. An archform may, in some instances, describe an averaged or smoothed arrangement of teeth in an arch.

An archform may be described by a set of control points, where each control point corresponds to some aspect of a tooth (e.g., to the centroid of the tooth, to the origin of the tooth's local coordinate system, to a landmark located within the anatomy of the tooth, etc.). Spline(s) may be fit through (e.g., a b-spline or a NURBS surface) the set of control points, which may approximate at least some aspect of the contour of the patient's dental arch. In some instances, aspects of the set of control points may be combined with aspects of the local coordinate axes of one or more teeth. For example, in the instance where the control points correspond to the teeth of the mandibular arch, the Z axis of a local tooth coordinate system may point downward relative to the patient's mouth (e.g., pointing in the gingival direction). A line segment may be defined between a point from the plurality of control points and a point which lies along the negative Z axis of a tooth, such that a first end point of the line segment lies in proximity to the control point, and the second end point of the line segment lies in proximity to the tooth's root. A 3D triangular mesh may be defined from the first and second end points of these line segments. A tooth may be modelled as being capable to slide along the upper and lower boundaries of that archfrom mesh (e.g., which may define a surface and/or a volume), as if the tooth was attached to a set of rails and constrained by the rails defined by the archform surface/volume. Additionally, the teeth are further constrained on this “rail” by requiring that the mesio-distal axis of each tooth's local coordinate system must be a tangent to the spline/occlusal surface of the rail at all times, thus ensuring the tooth is completely constrained on the rail/archform surface in all degrees of freedom. A tooth may move in a mesial direction or a distal direction, as long as the tooth runs along the rails. Movement in a tooth may effectuate or necessitate the movement of one or more other teeth along the rails (e.g., the neighbors of the moving tooth). In some instances, stochastic perturbations may be applied to the teeth along the rail, to adjust their positions and orientations, as long as the teeth remain attached to the rail. Machine learning models may be trained to move the teeth along the rail, such as neural networks that conform to architectures described herein.

FIG. 21 shows two archform meshes, one for each arch. As shown, FIG. 21 includes depictions of an upper archform mesh 2100 and a lower archform mesh 2102. Each mesh describes the “archform” of the arch, which may describe aspects of the arch (e.g., such as shape, structure and/or curves). Each control point 2108 may correspond to a point associated with a tooth. Each control point 2108 has one or more associated line segments 2106, which lies along the Z axis of the respective tooth local coordinate system. At the end of each of these line segments is a Z axis point 2104. For each of the upper 2100 and lower 2102 arches, a 3D triangle mesh is formed out of the control points 2108 and Z axis points 2104. In some instances, one or more points 2110 may be defined (one example of which is shown in FIG. 21), which may lie along a spline which is fitted through the control points 2108. In some instances, one or more points 2112 may be defined (one example of which is shown in FIG. 21), which may lie along a spline which is fitted through the Z axis points 2104 (which may be interpreted as an additional set of control points). In some implementations, the points shown in FIG. 21 may comprise a 3D triangle mesh, or other 3D representation.

In some instances, a machine learning model, such as a neural network, may be trained to adjust aspects of one or both of the rails (or some other aspect of the 3D archform mesh), such as the shape of one or more rails. These adjustments may be directed to produce favorable orthodontic treatment outcomes, such as aligning the teeth into poses which are suitable for a setup (e.g., an intermediate stage or a final setup). In some instances, a transform, such as those described herein, may be trained to effectuate changes to the shape of the archform mesh. In some instances, an autoencoder (such as a reconstruction autoencoder—an example of which is a variational autoencoder optionally utilizing normalizing flows) may be trained to effectuate changes to the shape of the archform mesh. The reconstruction autoencoder may be trained on a dataset of cohort patient cases, where each patient case contains at least one of: a set of tooth meshes, a mal transform for each tooth (to place the tooth in a maloccluded pose), an intermediate transform and an approved final setup transform. An archform mesh may be constructed in relation to a maloccluded arch, referred to herein as a mal archform mesh (or mal archform). An archform mesh may be constructed in relation to an approved final setup arch, referred to herein as a final setup archform mesh (or setup archform). An archform mesh may be constructed in relation to an intermediate stage arch, referred to herein as an intermediate stage archform mesh (or staging archform).

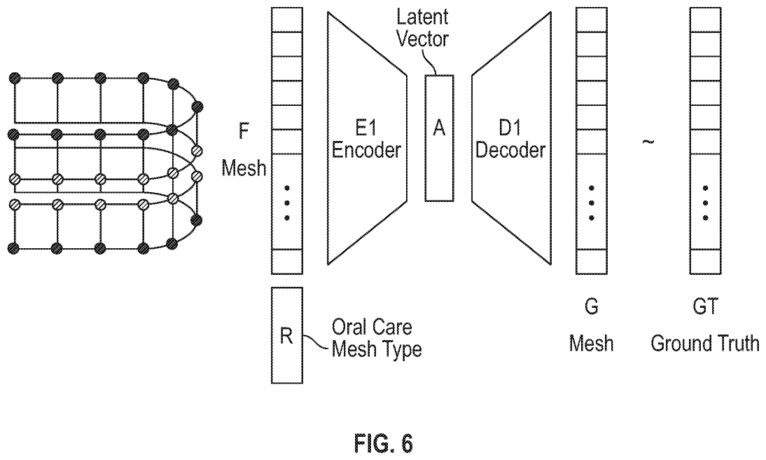

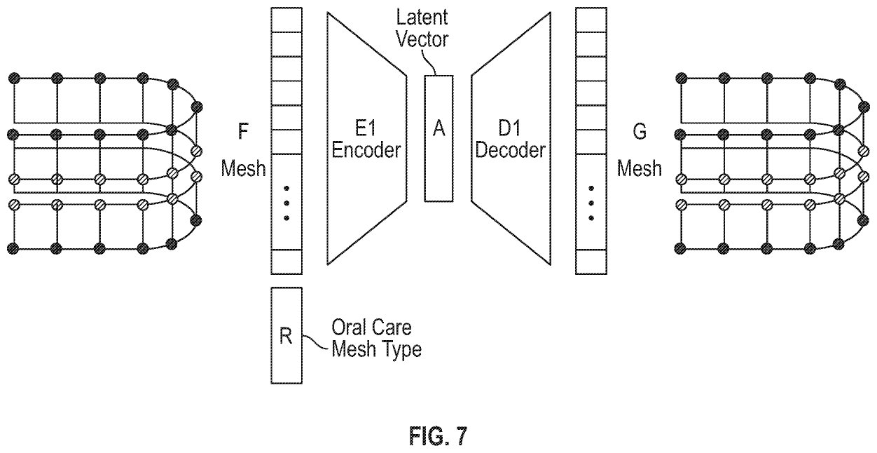

An autoencoder (such as that shown in FIG. 6 or FIG. 7) may be trained to reconstruct an archform mesh, such as a mal archform mesh. In some instances, the reconstruction autoencoder may encode an input archform mesh into a latent form using a multidimensional (e.g., 3D) encoder. This latent form of the archform mesh, which may reflect a reduced dimensionality form of the input archform mesh, may then be reconstructed into a facsimile of the input archform mesh. A reconstruction error may be computed, according to the descriptions herein, to quantify the difference in aspects of the input archform mesh and the reconstructed archform mesh (e.g., aspects such as shape). A low reconstruction error indicates that the 3D decoder performed effectively in reconstructing the input archform mesh. In some instances, the latent form of the archform mesh may undergo controlled modification (e.g., one or more numerical values of a latent vector may be adjusted). This adjustment may be performed in accordance with an understanding of the latent space, such as may be determined by a series of experiments which are designed to map out the latent space. In some instances, the latent space occupied by the latent form of the archform mesh may be mapped, so that a machine learning automation algorithm may determine the effect that a change in the latent form may have on the reconstructed archform mesh. Alternatively, the latent representation of the archform may be used as input to another machine learning/AI model to aid that model in efficiently learning to execute a different task. One or more controlled changes may be applied to the latent form, for the purpose of improving aspects of the reconstructed archform mesh (e.g., making the reconstructed archform mesh more nearly suitable for use in creating an oral care appliance—such as a clear tray aligner). The reconstructed archform mesh may be used to arrange teeth in a final setup (or alternatively, in an intermediate stage), for use in orthodontic treatment.

An autoencoder, such as a variational autoencoder (VAE), may be trained to encode 3D oral care representations (e.g., archform control points, 3D archform meshes, appliance components, polyline CTA trimlines, tooth restoration designs, and the like) into a latent space vector A, which may exist in an information-rich low-dimensional latent space. This latent space vector A may be particularly suitable for later processing by digital oral care applications, such as the modification of 3D oral care representations, because A enables complex oral care mesh data to be efficiently manipulated. Such an autoencoder may be trained to reconstruct the latent space vector A back into a facsimile of the inputted 3D oral care representation (e.g., a mesh of an archform). In some implementations, the latent space vector A may be strategically modified, so as to result in changes to the reconstructed mesh. In some instances, the reconstructed mesh may be a 3D oral care representation (e.g., an archform) with an altered and/or improved shape, such as would be suitable for use in the design of an oral care appliance, such as dental restoration appliance, such as a 3M® Filtek™ Matrix, a veneer or a clear tray aligner. The term “mesh” should be considered in a non-limiting sense to be inclusive of a 3D mesh, 3D point cloud, or a 3D voxelized representation.

A 3D oral care representation reconstruction VAE may advantageously make use of loss functions, nonlinearities (aka neural network activation functions) and/or solvers. Examples of loss functions may include one or more of: mean absolute error (MAE), mean squared error (MSE), L1-loss, L2-loss, KL-divergence, entropy, and/or reconstruction loss. Such loss functions enable each generated prediction to be compared against the corresponding ground truth value in a quantified manner, leading to one or more loss values which can be used to train, at least in part, one or more of the neural networks. Examples of solvers may include one or more of: dopri5, bdf, rk4, midpoint, adams, explicit adams, and/or fixed_adams. The solvers may enable the neural networks to solve systems of equations and corresponding unknown variables. Examples of nonlinearities may include one or more of: tanh, relu, softplus, elu, swish, square, and/or identity. The activation functions may be used to introduce nonlinear behavior to the neural networks in a manner that enables the neural networks to better represent the training data. Losses may be computed through the process of training the neural networks via backpropagation. Neural network layers such as one or more of the following may be used: ignore, concat, concat_v2, squash, concatsquash, scale, and/or concatscale.

In some implementations, an archform mesh reconstruction VAE model may be trained on ground truth archform mesh examples from cohort patient cases. In some implementations, an appliance component mesh (e.g., a parting surface or gingival trim mesh) reconstruction VAE model may be trained on ground truth appliance component examples from cohort patient cases. FIG. 6 shows a method of training such a VAE for the reconstruction of 3D oral care meshes. According to the training aspects shown in FIG. 6, a loss may be computed between the output G and ground truth GT, using the VAE loss calculation methods described herein. Backpropagation may be used to train E1 and D1 with such loss.

FIG. 7 shows the trained reconstruction VAE for 3D oral care meshes in deployment. According to the in-deployment method of FIG. 7, an oral care mesh reconstruction VAE is shown reconstructing a 3D archform mesh in deployment, where one or more aspects of the latent vector A have been altered, to effectuate improvements in aspects of the reconstructed oral care mesh (e.g., improvements to the shape and/or structure of a reconstructed archform mesh(es)).

Aspects of model training according to the techniques of this disclosure are described below. The 3D oral care representation reconstruction autoencoder (e.g., of which a variational autoencoder (VAE) and capsule autoencoder are non-limiting examples), may be trained to encode a tooth as a reduced-dimensionality form, called a latent space vector. The reconstruction VAE may be trained on example meshes of the particular 3D oral care mesh of interest (e.g., a 3D archform mesh). The input mesh may be received by the VAE, deconstructed into a latent space vector using a 3D encoder and then reconstructed into a facsimile of the input mesh using a 3D decoder. One advantage of this method is that the encoder E1 may become trained to encode an oral care mesh (e.g., an archform mesh, mesh of a dental appliance, tooth, gums, or other part of anatomy) into a reduced-dimension form that can be used in the training and deployment of an ML model for oral care mesh modification. This reduced-dimensionality form of the oral care mesh may be modified, and subsequently reconstructed into a reconstructed mesh with one or more aspects which have been altered to improve performance (e.g., the shape of an archform mesh may be altered so make the archform mesh more suitable for use in appliance creation). The shape of the oral care mesh may be altered to obtain technical improvements in terms of data precision.

The reconstructed oral care mesh may be compared to the input oral care mesh, for example using a reconstruction error, which may quantify the differences between the meshes. This reconstruction error may be computed using Euclidean distances between corresponding mesh elements between the two meshes. There are other methods of computing this error too which may be derived from material described elsewhere in this disclosure.

In some implementations, the mesh or meshes which are provided to the mesh reconstruction VAE my first be converted to vertex lists (or point clouds) before being provided to the encoder E1. This manner of handling the input to E1 may be conducive to either a single mesh input (such as in a tooth mesh classification task) or a set of multiple teeth (such as in the setups classification task). The input meshes need not be connected. Mesh element feature vectors may be computed for one or more mesh elements of the inputted 3D oral care representation. Such mesh element feature vectors may provide valuable information about the shape and/or structure of the input mesh to the reconstruction autoencoder (e.g., a variational autoencoder optionally utilising normalizing flows).

Aspects of the architecture of the models of this disclosure are described below. The encoder E1 may be trained to encode an oral care mesh into a latent space vector A (or “3D oral care representation vector”). In the course of the 3D oral care representation modification task, encoder E1 may arrange an input oral care mesh into a mesh element vector F, which may be encoded into a latent space vector A. This latent space vector A may be a reduced dimensionality representation of F that describes the important geometrical attributes of F. Latent space vector A may be provided to the decoder D1 to be restored to full resolution or near full resolution, along with the desired geometrical changes. The restored full resolution or near-full resolution mesh may be described by G, which may then be arranged into the reconstructed output mesh.

The performance of the mesh reconstruction VAE can be measured using reconstruction error calculations. In some examples, reconstruction error may be computed as element-to-element distances between two meshes, for example using Euclidean distances. Other distance measures are possible in accordance with various implementations of the techniques of this disclosure, such as Cosine distance, Manhattan distance, Minkowski distance, Chebyshev distance, Jaccard distance (e.g. intersection over union of meshes), Haversine distance (e.g., distance across a surface), and Sorensen-Dice distance.

The performance of a mesh reconstruction VAE may, in some implementations, be verified via reconstruction error plots and/or other key performance indicators. The latent space vectors for one or more input oral care meshes may be plotted (e.g., in 2D) using UMAP or t-SNE dimensionality reduction techniques and compared, to select the best available separability between classes of oral care mesh, indicating that the model has an awareness of the strong geometric variation between classes, and a strong similarity within a class. This would be illustrated by clear, non-overlapping clusters in the resulting UMAP/t-SNE plots.

In some instances, the latent vector corresponding to an oral care mesh may be used as a part of a classifier to classify that mesh (e.g., to identify a tooth type or to detect errors in the mesh or an arrangement of meshes, such as in a validation operation). The latent vector and/or computed mesh features (such as spatial and/or structural mesh features described herein) may be provided to a supervised machine learning model to classify the mesh. A non-limiting list of possible supervised ML models is found elsewhere in this disclosure.

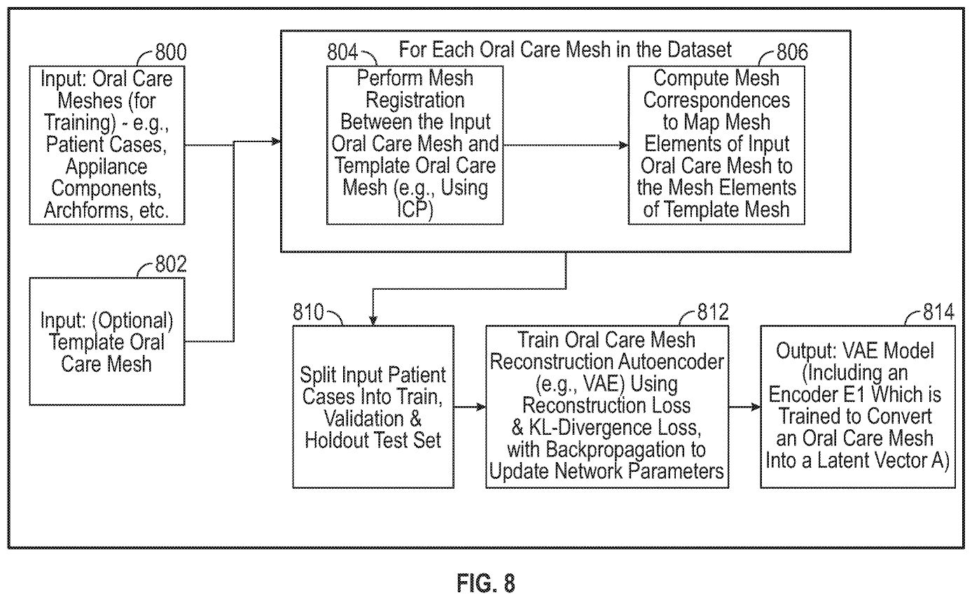

FIG. 6 shows a method that systems of this disclosure may implement to train an autoencoder for reconstructing an oral care mesh. The particular example of FIG. 7 illustrates training of a variational autoencoder (VAE) for reconstructing a 3D archform mesh. FIG. 8 describes additional steps in the training of a reconstruction autoencoder, according to techniques of this disclosure. An oral care mesh 800 (e.g., a 3D archform mesh) may be provided to the input of the method. The systems of this disclosure may perform a registration step (804) to align an oral care mesh with a template example 802 of that type of oral care mesh (e.g., using the iterative closest point technique), with the technical enhancement of improving the accuracy and data precision of the oral care mesh correspondence computation at 806. The systems of this disclosure may compute correspondences between an oral care mesh and the corresponding template oral care mesh, with the technical improvement of conditioning the oral care mesh to be ready to be provided to the reconstruction autoencoder. The dataset of prepared oral care meshes are split into train, validation and holdout test sets (810), which are then used to train a reconstruction autoencoder (812), described herein as an oral care mesh VAE, oral care mesh reconstruction VAE or more generally as a reconstruction autoencoder. The oral care mesh reconstruction VAE may comprise a 3D encoder which encodes an oral care mesh into a latent form (e.g., a latent vector A), and a subsequent 3D decoder which reconstructs that oral care mesh into a facsimile of the inputted oral care mesh. The oral care mesh reconstruction VAE of this disclosure may be trained using a combination of reconstruction loss and KL-Divergence loss, and optimally other of the loss functions described herein. The output of this method is a trained oral care mesh reconstruction VAE 814.

One of the steps which may take place in the VAE training data pre-processing is the calculation of mesh correspondences. Correspondences may be computed between the mesh elements of the input mesh and the mesh elements of a reference or template mesh with known structure. The goal of mesh correspondence calculation may be to find matching points between the surfaces of an input mesh and of a template (reference) mesh. Mesh correspondence may generate point to point correspondences between input and template meshes by mapping each vertex from the input mesh to at least one vertex in the template mesh. Correspondences may be computed between the mesh elements of the input mesh and the mesh elements of a reference or template mesh with known structure. In an example where an archform mesh is reconstructed, a range of entries in the vector may correspond to the portion of the archform proximate to the upper left first molar; another range of elements may correspond to the lower right central incisor; and so on. In some implementations, an input vector may be provided to the autoencoder (e.g., a vector of flags) which may define or otherwise influence the autoencoder as to which type of oral care mesh may have been received by the autoencoder as input. A data precision improvement of this approach of using mesh correspondences in mesh reconstruction is to reduce sampling error, improve alignment, and improve mesh generation quality. Further details on the use of mesh correspondences with the autoencoder models of this disclosure are found elsewhere in this disclosure.

In some implementations, an iterative closest point (ICP) algorithm may be run between the input oral care mesh and a template oral care mesh, during the computation of mesh correspondences. The correspondences may be computed to establish vertex-to-vertex relationships (between the input oral care mesh and the reconstructed oral care mesh), for use in computing reconstruction error.

Aspects of the training data for the models of this disclosure are described below. According to particular implementations, training data (e.g., for an archform) may be generalized to an arch or larger oral care representation or may be more specific to particular teeth within the larger oral care representation. In situations in which more specific training data is leveraged, the specific training data can be presented as an oral care mesh template. For instance, an oral care mesh template may be specific to one or more oral care mesh types. In some implementations, an oral care mesh template may be generated which is an average of many examples of a certain type of oral care mesh. In some implementations, an oral care mesh template may be generated which is an average of many examples of more than one oral care mesh type.

In some implementations, the pre-processing procedure may involve one or more of the following steps: registration to align the oral care mesh with a template oral care mesh (e.g., using ICP), and the computation of mesh correspondences (i.e., to generate mesh element-to-mesh element correspondences between the input oral care mesh and a template oral care mesh).

The encoder component E1 of in a fully trained mesh reconstruction autoencoder (e.g., for 3D archform meshes) may generate a latent vector A. The latent vector A may be a reduced-dimensionality representation of the input mesh (e.g., a 3D archform mesh). In some implementations, the latent vector A may be a vector of 128 real numbers (or in other examples consistent with this disclosure, some other size, such as 256 real numbers, 512 real numbers, etc.). The decoder D1 of the fully trained mesh reconstruction autoencoder may be capable to take the latent vector A as input and reconstruct a close facsimile of the input oral care mesh, with low reconstruction error. In some implementations, modifications may be made to the latent vector A, so as to effect changes in the shape of the reconstructed oral care mesh that is outputted from the decoder D1. Such modifications may be made after first mapping-out the latent space, to gain insight into the effects of making particular change. There may be a variety of loss functions which may be used in the training of E1 and D1, which may involve terms related to reconstruction error and/or KL-Divergence between distributions (e.g., in some instances to minimize the distance between the latent space distribution and a multidimensional Gaussian distribution). One purpose of the reconstruction error term is to compare the predicted reconstructed oral care mesh to the corresponding ground truth reconstructed oral care mesh. One purpose of the KL-divergence term may be to make the latent space more Gaussian, and therefore improve the quality of reconstructed meshes (i.e., especially in the case where the latent space vector may be modified, to change the shape of the outputted mesh, for example to modify an archform, modify an appliance component, modify a CTA trimline, modify a tooth restoration design, and the like).

In some implementations, the fully trained mesh reconstruction autoencoder may modify the latent vector A in a way that changes one or more characteristics of the reconstructed mesh. If the loss L is computed using only reconstruction error, and changes are made to the latent vector A, then in some use case scenarios, the reconstructed mesh may reflect the expected form of output (e.g., by being a recognizable archform mesh). In other use case scenarios, however, the output of the reconstructed mesh may not conform to the expected form of output (e.g., may not be a recognizable archform mesh).

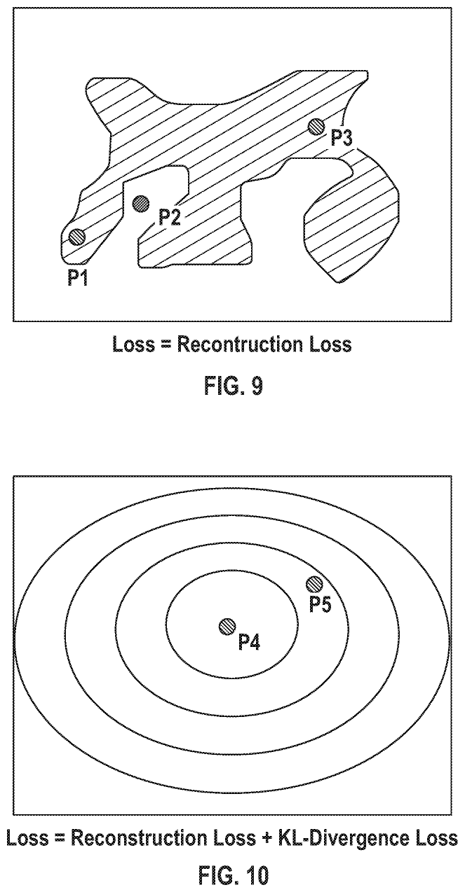

In FIG. 9, point P1 corresponds to the original form of a latent space vector A. The latent space of FIG. 9 is an example in which the loss incorporates reconstruction loss but does not incorporate KL-Divergence loss. Point P2 corresponds to a different location in the latent space, which may be sampled as a result of making modifications to the latent vector A, but where the oral care mesh which is reconstructed from P2 produces low quality output (e.g., output that does not look like a recognizable or otherwise suitable archform mesh). Point P3 corresponds to still a different location in the latent space, which may be sampled as a result of making a different set of modifications to the latent vector A, but where the oral care mesh which is reconstructed from P3 provides good output (e.g., having the appearance of an archform mesh design which is suitable for use in creating a final setup—such as with a setups prediction neural network). In the case where the loss L involves only reconstruction error, the subset of the latent space which can be sampled to produce a latent space vector P3 which yielding a valid reconstructed oral care mesh may be irregular and hard to predict.

A loss calculation may, in some implementations, incorporate normalizing flows, for example, by the incorporation of a KL-divergence term. FIG. 10 illustrates a latent space in which the loss includes both reconstruction loss and KL-divergence loss. If the loss is improved by incorporating a KL-divergence term, the quality of the latent space may improve significantly. The latent space may become more Gaussian under this new scenario (as shown in FIG. 10), where a latent supervector A corresponds to point P4 near the center of a multidimensional Gaussian curve. Changes can be made to the latent supervector A, yielding point P5 that is positioned nearby to P4, where the resulting reconstructed mesh is highly likely to reflect desired attributes (e.g., is highly likely to be a valid archform mesh). The introduction of the KL-divergence term to loss may improve the reliability of the process of modifying the latent space vector A and obtaining a valid reconstructed oral care mesh. In some implementations, as with a capsule autoencoder, the trained model of this disclosure may use a latent capsule instead of the latent vector and may modify and reconstruct the latent capsule for mesh reconstruction according to the aspects of this disclosure.

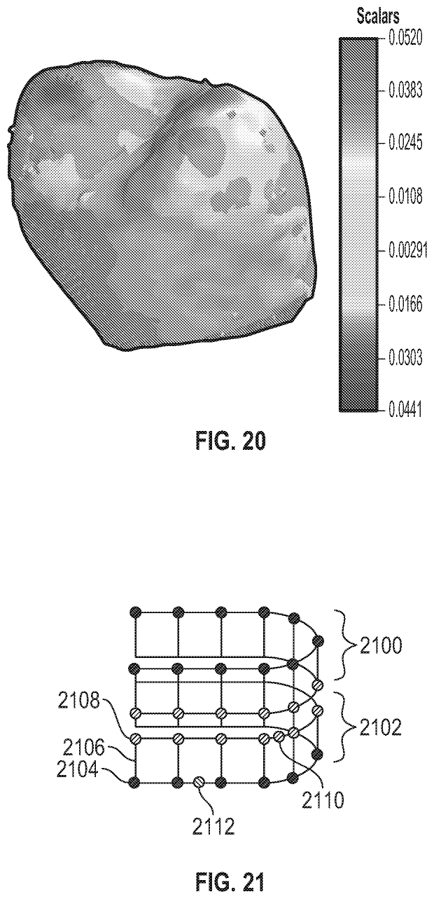

FIG. 20 depicts the reconstruction error for a tooth which has been reconstructed by a tooth reconstruction autoencoder (e.g., which has been trained, at least in part, using the mesh reconstruction autoencoder loss calculation methods described herein). FIG. 20 shows a “reconstruction error plot” with units in millimeters (mm). Notice that the reconstruction error is less than 50 microns at the cusp tips, and much less than 50 microns over most of the tooth surface. Compared to a typical tooth with a size of 1.0 cm, an error rate of 50 microns (or less) means that the tooth surface was reconstructed with an error rate of less than 0.5%.

For a given domain (e.g., oral care mesh modification), the latent space can be mapped out, so that changes to the latent space vector A may lead to relatively well-reconstructed meshes. The latent space may be systematically mapped by generating latent vectors with preselected or predetermined variations in value (e.g., by experimenting with different combinations of 128 values in an example latent vector). In some instances, a grid search of values may be performed, providing the advantage of efficiently exploring the latent space. With the latent space mapped out, the shape of an oral care mesh may be modified by incrementally moving (or “nudging”) the values in one or more elements of the latent vector values towards the portion of the mapped out latent space which has been found to correspond to the desired oral care mesh characteristics. The use of KL-divergence in the loss calculation increases the likelihood that the modified latent vector is reconstructed into a valid example of the inputted 3D representation (e.g., 3D archform mesh).

The modifications to a latent vector may, in some implementations, be carried out via an ML model, such as one of the neural network models or other ML models disclosed elsewhere in this disclosure. In some implementations, a neural network may be trained to operate within the latent space representation comprising such vectors A of oral care meshes. The mapping of the latent space of A may represent a previously generated mapping based on applying controlled adjustments to trial latent vectors and observing the resulting changes to the resulting reconstructed oral care meshes (e.g., after the modified A has been reconstructed back into a full mesh or meshes). The mapping of the latent space may, in some instances, follow methodical search patterns, such as in the case of implementing a grid search.

In some implementations, an oral care reconstruction VAE of this disclosure may take a single input of oral care mesh name/type/designation R. R may function to influence the VAE to output an oral care mesh of the designated type (e.g., an archform mesh or an appliance component, such as a parting surface). This can be accomplished by generating a latent vector A′ for use in reconstructing a suitable oral care mesh. In some implementations, this latent vector A′ may be sampled or generated “on the fly”, out of an existing or prior mapping of the latent vector space. Such a mapping may be performed to provide an understanding of which portions of the latent vector space correspond to different shapes, structures and/or geometries of an oral care mesh. For example, out of the 128 real values in an example of latent vector A′ (while it will be appreciated that other sizes are possible in accordance with this disclosure), certain elements, and in some cases, certain ranges of values, for those vector elements may be determined to correspond to a certain type/name/designation of oral care mesh and/or an oral care mesh with a certain shape or other intended characteristics. This model for oral care mesh generation may also apply to the generation of oral care hardware, appliances, and/or appliance components (such as to be used for orthodontic treatment). This model may also be trained for the generation of dental anatomy, such as tooth crowns and/or roots. This model may also be trained for the generation of other types on non-oral care meshes as well.

Systems of this disclosure may compute reconstruction error as a combination of L1 loss and MSE loss, as shown in the following line of pseudocode: reconstruction_error=0.5*L1(all_points_target,all_points_predicted)+0.5*MSE(all_points_target,all_points_predicted). In the above example, all_points_target may comprise a point cloud corresponding to a ground truth tooth restoration design (or a ground truth example of some other 3D oral care representation). The “all_points_predicted” variable may comprise a point cloud corresponding to a generated example of a tooth restoration design (or a generated example of some other kind of 3D oral care representation).

The following is one possible experiment to gain insight into link between changes to the latent vector and the resulting effects on the reconstructed tooth mesh.

For each dimension of the latent space (e.g., for each cell in the latent vector of, for example, 128 cells (although 64-cell, 512-cell, 1024-cell, and other differently sized latent vectors are possible), take five (5) datapoints (0,1,2,3,4) with point 2 centered on the mean of that dimension (i.e., a value of 0, in the center of the Gaussian distribution). Execute a trained encoder-decoder structure (e.g., a transformer, or a VAE or a Capsule Autoencoder which has been trained for tooth reconstruction) to reconstruct a tooth mesh using a latent vector of all zeros. This is the “default” or “average” tooth of this experiment. Then generate 2 samples at each side of this dimension's distribution (datapoints 0,1,3,4 from above). These may, for example, be located at 1, 1.5, or 2 standard deviations from the center of this dimension's distribution at each side (positive and negative), enabling the experimenter to gain insight into which aspects of the reconstructed tooth correspond to this dimension of the latent vector. Any one of these dimensions may impact multiple aspects of the reconstructed tooth. In some instances, linear algebra may be used to isolate independent features, by identifying the vector dimensions which most greatly impact a particular aspect of the reconstructed tooth shape and/or structure.

In some instances, the trained encoder-decoder structures (e.g., reconstruction autoencoders or transformers) of this disclosure may cluster reconstructed teeth to gain and/or provide insight into their relationships, and to help in understanding the link between changes to the latent vector and the characteristics of the reconstructed tooth. If the mesh (A) is the input to the reconstruction autoencoder (or transformer), and mesh (B) is the reconstructed tooth that is generated by the tooth reconstruction autoencoder (or transformer). By generating latent codes for both A and B, a vector may be computed that moves from A into B (e.g., B-A). A large set of such mapping vectors may be compiled and used to identify the dominant sub-vector(s) responsible for pushing a point in latent vector space towards “generalized” restored tooth characteristics. Such a mapping vector may then be added to the latent vector for a tooth to generate a reconstructed tooth mesh with the intended shape and/or structural characteristics. In some instances, a viewer of this disclosure may provide a view of three (3) or five (5) meshes at once, and store images of these teeth at various orientations for offline analysis and comparisons. Although this experiment uses five (5) data points, any other number of data points may be used in other experiments.

Although the preceding example method discusses modifications to the latent vector of a tooth, it should be understood, without the loss of generality, that the method may also apply to other 3D representations within the scope of this disclosure. For example, the latent vectors of any of the following may be mapped-out: tooth restoration design, other aspects of the patient's dentition, fixture model or fixture model component, appliance, oral care appliance component, or the like. In some implementations, a latent vector (or other latent representation) may undergo these modifications as a part of a Latent Representation Modification Module (LRMM). Oral care arguments may be provided to the LRMM to influence that module to perform modifications to the latent vector, to influence the resulting generated (or modified) 3D representation.

An autoregressive generative machine learning model of this disclosure may be trained to impute one or more missing or incomplete aspects of a 3D oral care representation. For example, the model may be trained to add one or more mesh elements to a 3D oral care representation to fill in holes, fill in rough edges or boundaries, or compete missing portions of the shape or structure of a 3D oral care representation). A transformer of this disclosure may be trained for this autoregressive behavior, as well. For example, a transformer may be trained to fill in one or more missing mesh elements (e.g., vertices, points, edges, faces or voxels) in a 3D representation (e.g., mesh) of a tooth crown, which may have holes or other missing aspects as a result of the intraoral scanning process (e.g., a portion of the tooth crown may have been blocked by an adjacent tooth or occluded by hardware in the mouth). This mesh completion (or mesh fill-in or mesh element imputation) technique may be integrated with an appliance generation method, for example, to clean up tooth crown (or root) meshes after segmentation and before further appliance generation steps (e.g., coordinate system prediction, appliance component generator and/or placement, setups prediction, restoration design generation, or the like). A generative machine learning model (e.g., such as a transformer trained for mesh element in-filling) may estimate joint distributions over mesh elements from a training dataset of past cohort patient case data (e.g., tooth mesh data or some other type of 3D oral care representation) in accordance with aspects of this disclosure.

Techniques described herein may be trained to generate 3D oral care representations (e.g., tooth restoration designs, appliance components, and other examples of 3D oral care representations described herein). Such 3D representations may comprise point clouds, polylines, meshes, voxels and the like. Such 3D oral care representation may be generated according to the requirements of the oral care arguments which may, in some implementations, be supplied to the generative model. Oral care arguments may include oral care parameters as disclosed herein, or other real-valued, text-based or categorical inputs which specify intended aspects of the one or more 3D oral care representations which are to be generated. In some instances, oral care arguments may include oral care metrics, which may describe intended aspects of the one or more 3D oral care representations which are to be generated. Oral care arguments are specifically adapted to the implementations described herein. For example, the oral care arguments may specify the intended the designs (e.g., including shape and/or structure) of 3D oral care representations which may be generated (or modified) according to techniques described herein. In short, implementations using the specific oral care arguments disclosed herein generate more accurate 3D oral care representations than implementations that do not use the specific oral care arguments. In some instances, a text encoder may encode a set of natural language instructions from the clinician (e.g., generate a text embedding). A text string may comprise tokens. An encoder for generating text embeddings may, in some implementations, apply either mean-pooling or max-pooling between the token vectors. In some instances, a transformer (e.g., BERT or Siamese BERT) may be trained to extract embeddings of text for use in digital oral care (e.g., by training the transformer on examples of clinical text, such as those given below). In some instances, such a model for generating text embeddings may be trained using transfer learning (e.g., initially trained on another corpus of text, and then receive further training on text related to digital oral care). Some text embeddings may encode text at the word level. Some text embeddings may encode text at the token level. A transformer for generating a text embedding may, in some implementations, be trained, at least in part, with a loss calculation which compares predicted outputs to ground truth outputs (e.g., softmax loss, multiple negatives ranking loss, MSE margin loss, cross-entropy loss or the like). In some instances, the non-text arguments, such as real values or categorical values, may be converted to text, and subsequently embedded using the techniques described herein. The following are examples of natural language instructions that may be issued by a clinician to the generative models described herein: “Generate a restoration design (alternatively a veneer design) which closes the diastema between tooth #8-9 by evenly adding width onto the mesial of both teeth”, “Generate a restoration design (alternatively a veneer design) which ensures that the incisal edges of #6-11 form an even semicircle with the incisal edges of the posterior teeth (when looking from the incisal view)”, or “Generate a customized crown for an upper left central incisor, to be implanted. The crown shape should take into consideration the shape of adjacent teeth and should have no more than x mm (e.g.: 0.1 mm) space between the adjacent teeth.”

Techniques of this disclosure may, in some implementations, use PointNet, PointNet++, or derivative neural networks (e.g., networks trained via transfer learning using either PointNet or PointNet++ as a basis for training) to extract local or global neural network features from a 3D point cloud or other 3D representation (e.g., a 3D point cloud describing aspects of the patient's dentition—such as teeth or gums). Techniques of this disclosure may, in some implementations, use U-Nets to extract local or global neural network features from a 3D point cloud or other 3D representation.

3D oral care representations are described herein as such because 3-dimensional representations are currently state of the art. Nevertheless, 3D oral care representations are intended to be used in a non-limiting fashion to encompass any representations of 3-dimensions or higher orders of dimensionality (e.g., 4D, 5D, etc.), and it should be appreciated that machine learning models can be trained using the techniques disclosed herein to operate on representations of higher orders of dimensionality.

In some instances, input data may comprise 3D mesh data, 3D point cloud data, 3D surface data, 3D polyline data, 3D voxel data, or data pertaining to a spline (e.g., control points). An encoder-decoder structure may comprise one or more encoders, or one or more decoders. In some implementations, the encoder may take as input mesh element feature vectors for one or more of the inputted mesh elements. By processing mesh element feature vectors, the encoder is trained in a manner to generate more accurate representations of the input data. For example, the mesh element feature vectors may provide the encoder with more information about the shape and/or structure of the mesh, and therefore the additional information provided allows the encoder to make better-informed decisions and/or generate more-accurate latent representations of the mesh. Examples of encoder-decoder structures include U-Nets, autoencoders or transformers (among others). A representation generation module may comprise one or more encoder-decoder structures (or portions of encoders-decoder structures—such as individual encoders or individual decoders). A representation generation module may generate an information-rich (optionally reduced-dimensionality) representation of the input data, which may be more easily consumed by other generative or discriminative machine learning models.

A U-Net may comprise an encoder, followed by a decoder. The architecture of a U-Net may resemble a U shape. The encoder may extract one or more global neural network features from the input 3D representation, zero or more intermediate-level neural network features, or one or more local neural network features (at the most local level as contrasted with the most global level). The output from each level of the encoder may be passed along to the input of corresponding levels of a decoder (e.g., by way of skip connections). Like the encoder, the decoder may operate on multiple levels of global-to-local neural network features. For instance, the decoder may output a representation of the input data which may contain global, intermediate or local information about the input data. The U-Net may, in some implementations, generate an information-rich (optionally reduced-dimensionality) representation of the input data, which may be more easily consumed by other generative or discriminative machine learning models.

An autoencoder may be configured to encode the input data into a latent form. An autoencoder may train an encoder to reformat the input data into a reduced-dimensionality latent form in between the encoder and the decoder, and then train a decoder to reconstruct the input data from that latent form of the data. A reconstruction error may be computed to quantify the extent to which the reconstructed form of the data differs from the input data. The latent form may, in some implementations, be used as an information-rich reduced-dimensionality representation of the input data which may be more easily consumed by other generative or discriminative machine learning models. In most scenarios, an autoencoder may be trained to input a 3D representation, encode that 3D representation into a latent form (e.g., a latent embedding), and then reconstruct a close facsimile of that input 3D representation as the output.

A transformer may be trained to use self-attention to generate, at least in part, representations of its input. A transformer may encode long-range dependencies (e.g., encode relationships between a large number of inputs). A transformer may comprise an encoder or a decoder. Such an encoder may, in some implementations, operate in a bi-directional fashion or may operate a self-attention mechanism. Such a decoder may, in some implementations, may operate a masked self-attention mechanism, may operate a cross-attention mechanism, or may operate in an auto-regressive manner. The self-attention operations of the transformers described herein may, in some implementations, relate different positions or aspects of an individual 3D oral care representation in order to compute a reduced-dimensionality representation of that 3D oral care representation. The cross-attention operations of the transformers described herein may, in some implementations, mix or combine aspects of two (or more) different 3D oral care representations. The auto-regressive operations of the transformers described herein may, in some implementations, consume previously generated aspects of 3D oral care representations (e.g., previously generated points, point clouds, transforms, etc.) as additional input when generating a new or modified 3D oral care representation. The transformer may, in some implementations, generate a latent form of the input data, which may be used as an information-rich reduced-dimensionality representation of the input data, which may be more easily consumed by other generative or discriminative machine learning models.

In some implementations, an encoder-decoder structure may first be trained as an autoencoder. In deployment, one or more modifications may be made to the latent form of the input data. This modified latent form may then proceed to be reconstructed by the decoder, yielding a reconstructed form of the input data which differs from the input data in one or more intended aspects. Oral care arguments, such as oral care parameters or oral care metrics may be supplied to the encoder, the decoder, or may be used in the modification of the latent form, to influence the encoder-decoder structure in generating a reconstructed form that has desired characteristics (e.g., characteristics which may differ from that of the input data).

Techniques of this disclosure may, in some instances, be trained using federated learning. Federated learning may enable multiple remote clinicians to iteratively improve a machine learning model (e.g., validation of 3D oral care representations, mesh segmentation, mesh cleanup, other techniques which involve labeling mesh elements, coordinate system prediction, non-organic object placement on teeth, appliance component generation, tooth restoration design generation, techniques for placing 3D oral care representations, setups prediction, generation or modification of 3D oral care representations using autoencoders, generation or modification of 3D oral care representations using transformers, generation or modification of 3D oral care representations using diffusion models, 3D oral care representation classification, imputation of missing values), while protecting data privacy (e.g., the clinical data may not need to be sent “over the wire” to a third party). Data privacy is particularly important to clinical data, which is protected by applicable laws. A clinician may receive a copy of a machine learning model, use a local machine learning program to further train that ML model using locally available data from the local clinic, and then send the updated ML model back to the central hub or third party. The central hub or third party may integrate the updated ML models from multiple clinicians into a single updated ML model which benefits from the learnings of recently collected patient data at the various clinical sites. In this way, a new ML model may be trained which benefits from additional and updated patient data (possibly from multiple clinical sites), while those patient data are never actually sent to the 3rd party. Training on a local in-clinic device may, in some instances, be performed when the device is idle or otherwise be performed during off-hours (e.g., when patients are not being treated in the clinic). Devices in the clinical environment for the collection of data and/or the training of ML models for techniques described herein may include intra-oral scanners, CT scanners, X-ray machines, laptop computers, servers, desktop computers or handheld devices (such as smart phones with image collection capability). In addition to federated learning techniques, in some implementations, contrastive learning may be used to train, at least in part, the ML models described herein. Contrastive learning may, in some instances, augment samples in a training dataset to accentuate the differences in samples from difference classes and/or increase the similarity of samples of the same class.

In some instances, a local coordinate system for a 3D oral care representation, such as a tooth, may be described by one or more transforms (e.g., an affine transformation matrix, translation vector or quaternion). Systems of this disclosure may be trained for coordinate system prediction using past cohort patient case data. The past patient data may include at least: one or more tooth meshes or one or more ground truth tooth coordinate systems. Machine learning models such as: U-Nets, encoders, autoencoders, pyramid encoder-decoders, transformers, or convolution and/or pooling layers, may be trained for coordinate system prediction. Representation learning may determine a representation of a tooth (e.g., encoding a mesh or point cloud into a latent representation, for example, using a U-Net, encoder, transformer, convolution and/or pooling layers or the like), and then predict a transform for that representation (e.g., using a trained multilayer perceptron, transformer, encoder, transformer, or the like) that defines a local coordinate system for that representation (e.g., comprising one or more coordinate axes). In the instance where the coordinate system is predicted for a tooth mesh, the mesh convolutional techniques described herein can leverage invariance to rotations, translations, and/or scaling of that tooth mesh to generate predications that techniques that are not invariant to the rotations, translations, and/or scaling of that tooth mesh cannot generate. Pose transfer techniques may be trained for coordinate system prediction, in the form of predicting a transform for a tooth. Reinforcement learning techniques may be trained for coordinate system prediction, in the form of predicting a transform for a tooth.

Machine learning models such as: U-Nets, encoders, autoencoders, pyramid encoder-decoders, transformers, or convolution and/or pooling layers, may be trained as a part of a method for hardware (or appliance component) placement. Representation learning may train a first module to determine an embedded representation of a 3D oral care representation (e.g., encoding a mesh or point cloud into a latent form using an autoencoder, or using a U-Net, encoder, transformer, block of convolution and/or pooling layers or the like). That representation may comprise a reduced dimensionality form and/or information-rich version of the inputted 3D oral care representation. In some implementations, the generation of a representation may be aided by the calculation of a mesh element feature vector for one or more mesh elements (e.g., each mesh element). In some implementations, a representation may be computed for a hardware element (or appliance component). Such representations are suitable to be provided to a second module, which may perform a generative task, such as transform prediction (e.g., a transform to place a 3D oral care representation relative to another 3D oral care representation, such as to place a hardware element or appliance component relative to one or more teeth) or 3D point cloud generation. Such a transform may comprise an affine transformation matrix, translation vector or quaternion or the like. Machine learning models which may be trained to predict a transform to place a hardware element (or appliance component) relative to elements of patient dentition include: MLP, transformer, encoder, or the like. Systems of this disclosure may be trained for 3D oral care appliance placement using past cohort patient case data. The past patient data may include at least: one or more ground truth transforms and one or more 3D oral care representations (such as tooth meshes, or other elements of patient dentition). In the instance where a U-Net (among other neural networks) is trained to generate the representations of tooth meshes, the mesh convolution and/or mesh pooling techniques described herein leverage invariance to rotations, translations, and/or scaling of that tooth mesh to generate predications that techniques that are not invariant to the rotations, translations, and/or scaling of that tooth mesh cannot generate. Pose transfer techniques may be trained for hardware or appliance component placement. Reinforcement learning techniques may be trained for hardware or appliance component placement.

Techniques of this disclosure may be trained to generate point clouds (e.g., where a point may be described as a 1D vector—such as (x, y, z)), polylines (points connected in order by edges), meshes (points connected via edges to form faces), splines (which may be computed through a set of generated control points), sparse voxelized representations (which may be described as a set of points corresponding to the centroid of each voxel or to some other landmark of the voxel—such as the boundary of the voxel), a transform (which may take the form of one or more 1D vectors or one or more 2D matrices—such as a 4×4 matrix) or the like. In some implementations, a voxelized representation may be computed from a 3D point cloud or a 3D mesh. In some implementations, a 3D point cloud may be computed from a voxelized representation. In some implementations, a 3D mesh may be computed from a 3D point cloud.

Disclosed herein is a recursive inference (RI) model for 3D oral care representation generation (or modification). The RI model may be trained on a training dataset of a particular type of 3D oral care representation, examples of which are described herein. The RI model may then be used in deployment to generate (or modify) examples of that particular types of 3D oral care representation). 3D oral care representations which may be generated or modified (based on respective training data) include 1) tooth restoration designs, 2) fixture model designs (e.g., comprising one or more fixture model components or one or more aspects of the patient's dentition), 3) oral care appliance components (e.g., generated components), 4) setups transforms for teeth, 5) transforms for pre-fab library components for oral care appliances, 6) archforms, 7) clear tray aligner trimlines (e.g., for trimming an aligner tray from a printed fixture model), 8) a set of mesh element labels for use in segmentation or mesh cleanup, or other of the 3D oral care representations described herein. For example, an RI model to generate (or modify) tooth restoration designs may be trained on tooth restoration design data. Furthermore, an RI model to generate (or modify) a digital fixture model design may be trained on tooth restoration design data (e.g., that contains aspects of dentition, or one or more fixture model components—as described herein). In yet another example, an RI model to generate (or modify) tooth transforms for setups prediction may be trained on tooth mesh and transform data (e.g., mal transforms for the teeth, along with ground truth reference transforms for final setups or intermediate stages—to be used in loss calculation).

Archforms may be described by control points (with splines) or polylines. Trimlines may be described by polylines (or control points and/or splines). Tooth restoration designs, fixture models or generated appliance components may be described by 3D meshes, 3D point clouds, 3D voxelized representations, or the like. Transforms may be described by transformation matrices or vectors, translation vectors, quaternions or other data structures described herein.

Techniques of this disclosure offer technical improvements over existing systems and techniques. For instance, techniques of this disclosure offer technical improvements to the technical problem of generating (or modifying) 3D oral care representations for use in generating oral care appliances, particularly to the introduction of mesh element features, oral care metrics or oral care parameters.

Furthermore, in some implementations, techniques of this disclosure may be trained to generate other kinds of data representations which are not contemplated by existing systems and techniques, including transforms, coordinate system axes (e.g., for teeth or other aspects of the patient's dentition), or mesh element labels to name two examples. Techniques of this disclosure may be trained on oral care data (e.g., meshes, point clouds or voxelized representations which describe dental anatomy or appliance components, transforms which place teeth or appliance components into poses which are suitable for clinical treatment, mesh element labels which may be defined for use with segmentation or mesh cleanup operations; or other examples of 3D oral care representations described herein) to generate 3D oral care representations which are suited to the generation of oral care appliances.

During either training or deployment, the techniques of this disclosure may take as input a 3D oral care representation, which may be encoded to a latent form or latent representation by a first ML module (e.g., an encoder). The first ML module may, in some implementations, be trained, at least in part, by the calculation of a reconstruction loss (e.g., a cross-entropy loss), which may compare a generated output to a ground truth reference. When mesh, voxel or point cloud data (or data describing other 3D representations) are processed by the techniques of this disclosure, a technical improvement is realized by the calculation of mesh element feature vectors for one or more mesh elements. Such mesh element feature vectors may be computed by a mesh element feature module 1102. Such mesh element feature vectors may be provided to the first ML module and may improve the accuracy or fidelity of the latent form which is generated by that first ML module. The improved accuracy of the latent form may enable the latter generative steps to output an improved generated (or modified) result (e.g., a 3D representation of a tooth for restoration, a set of transforms for use in orthodontic setups generation, one or more coordinate system axes, or mesh element labels for use in segmentation or in mesh cleanup).

Further technical improvements are realized by techniques of this disclosure by the use of oral care parameters (e.g., which may specify customized characteristics of an intended 3D oral care representation which is to be generated or modified) or oral care metrics (e.g., which may quantify or measure physical aspects of one or more teeth; which may quantify the shape and/or structure of an individual tooth or appliance component; or may quantify the poses and/or physical arrangements between two or more teeth or appliance components), collectively referred to as oral care arguments. Oral care arguments are specifically adapted to the implementations described herein. For example, the oral care arguments may specify the intended the designs (e.g., including shape and/or structure) of 3D oral care representations which may be generated (or modified) according to techniques described herein. In short, implementations using the specific oral care arguments disclosed herein generate more accurate 3D oral care representations than implementations that do not use the specific oral care arguments. Oral care metrics 1108 may be computed for a training example (e.g., the set of the patient's teeth) and be provided to either of the model training or model deployments methods of the RI model, which generates the augmented feature grid AFGₜ. Such oral care metrics improve the augmented feature grid AFGₜ by quantifying specific key aspects of the inputted 3D oral care representation 1100 (e.g., by quantifying the shapes of one or more teeth or appliance components, or by measuring the special relationships between one or more teeth or appliance components) that the Generator module 1124 may use to generate customized output which is suitable for use in clinical treatment (e.g., generating customized tooth restoration designs, customized appliance components, customized orthodontic setups transforms, coordinate axes, or the like). The oral care metrics 1108 may be provided to the generator module 1124 at training time, to teach the generator module 1124 about the shape and/or structure of the provided input data. After deployment, values which are of the same format as the oral care metrics may be provided to the generator module 1124 to influence the generator module 1124 to generate the intended shape and/or structure of the 3D oral care representation which is intended to appear at the output of the generator 1124 (e.g., a new geometry or a modified version of an inputted geometry). The generator module 1124 may be trained, at least in part, by a loss which compares predicted to ground truth reference outputs.

A 3D representation (e.g., a 3D point cloud) may be generated (or modified), at least in part, using one or more neural networks (e.g., including one or more transformers or including one or more autoencoders) which have been trained for 3D point cloud generation (or modification). Such a transformer may, in some implementations, be used in a recursive inference procedure, which may recursively refine the shape and/or structure of a 3D mesh (or other 3D representation, such as a voxelized representation). In some implementations, the techniques may be used in a recursive inference procedure, which may recursively refine the shape of a 3D point cloud. Such a point cloud may be based on an existing 3D oral care representation which requires modification, or such a point cloud may correspond to a newly generated or newly initialized example. Such an architecture may also generate (or modify) other types of data structures, such as transforms (e.g., a matrix to define rotation, translation, or scaling) or vectors (e.g., a coordinate tuple which may define a point or the like). Such an RI model may be trained on cohort patient case data, which may comprise one or more 3D oral care representations, as defined herein. Such a model may be trained to learn the distribution of such training data and generate new examples of 3D oral care representations which are suitable for clinical use (e.g., suitable for use in generating a tooth restoration design, an appliance component, a trimline, an archform, a transform, or the like).

Techniques described herein may, in some implementations, be trained to generate transforms. Such transforms may comprise transformation matrices or vectors, quaternions, or other data structures disclosed herein. Such transforms may place teeth or appliance components relative to elements of the patient's dentition (e.g., such as in appliance design or setups prediction). In some implementations, the techniques may be trained to generate (or modify) mesh element labels (e.g., for use in labelling mesh elements as a part of segmentation or mesh cleanup operations). In some implementations, the techniques may be trained to generate (or modify) tooth coordinate systems (e.g., which may be used by setups prediction models in the placement of teeth).

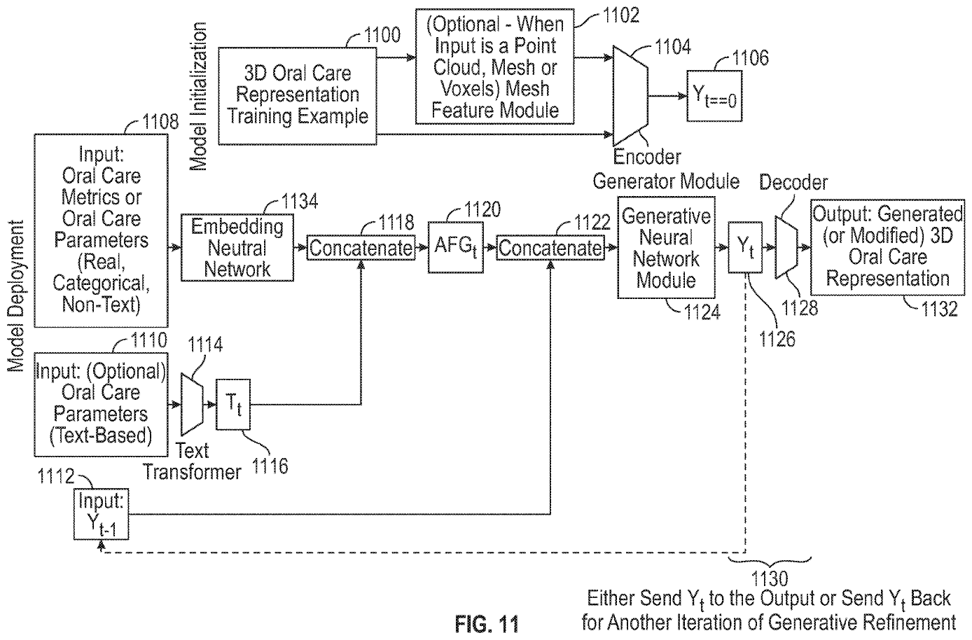

As seen in the deployment method of FIG. 11, an example 1100 from the training dataset may be provided to an optional mesh element feature module 1102, which may compute mesh element feature vectors. The training example 1100 (with optional mesh element feature vectors for its mesh elements) may be provided to encoder 1104, which may generate an initial latent representation Yₜ₌₌₀ 1106. The latent representation Yₜ₌₌₀ 1106 may be used as the initial value of the input representation Yₜ₋₁ 1112 (aka latent representation from the prior iteration of the recursive inference model). Yₜ₋₁ 1112 may be concatenated 1122 with AFGₜ 1120 and provided to generator module 1124. Oral care arguments 1108 may be concatenated 1118 with text representation Tₜ 1116, forming an augmented feature grid AFGₜ 1120. The text representation Tₜ 1116 may be generated by providing text-based oral care arguments 1110 to text transformer encoder 1114. In some implementations, an augmented feature grid AFGₜ 1120 may incorporate at least some aspect of an oral care argument 1108, as described herein, which may enable customization of a 3D representation which is generated (or modified) using techniques of this disclosure (e.g., a transformer which implements a recursive inference procedure). In some implementations, an augmented feature grid AFGₜ may incorporate at least some aspects of an oral care metric 1108 or an oral care parameter 1108. Such an oral care metric or an oral care parameter may indicate an intended outcome of 3D representation generation (or modification) using the techniques of this disclosure. An example of an oral care parameter is “Alignment,” which may influence the techniques of this disclosure in the generation of orthodontic setups transforms for setups prediction (e.g., final setups or intermediate staging). A further example of an oral parameters is “Height of Contour,” which may influence the techniques of this disclosure in the generation of a tooth restoration design (e.g., for use in generating a crown, veneer or a tooth restoration appliance).

In some implementations, an augmented feature grid AFGₜ 1120 may incorporate at least some aspect of a text input 1110. Text input 1110 may contain instructions in natural language pertaining to the intended design or other intended aspects of a 3D oral care representation which is to be generated (or modified). Text input 1110 may use a text transformer encoder (e.g., which may contain one or more BERT modules—Bidirectional Encoder Representations from Transformers) 1114 to encode a textual description into reorganized or latent form Tₜ 1116. In some implementations, Tₜ may be combined or concatenated 1118 with one or more oral care arguments (or with a latent representation of one or more oral care arguments). For example, latent for T and one or more oral care arguments may be combined using matrix concatenation. In some implementations, such oral care arguments may first undergo encoding as a latent embedding, for example, using an encoder which has been trained for that purpose. Latent form Tₜ 1116 (in combination with any oral care arguments or latent-encoded oral care arguments) may be projected into an augmented feature grid AFGₜ 1120. A probability distribution of an augmented latent feature codes Yₜ 1126 may be defined, and iteratively refined through the training process. A current augmented feature grid AFGₜ 1120 of a training example may be concatenated 1122 (or otherwise combined) with a augmented feature grid distribution from the prior timestep of training Yₜ₋₁ 1112 (e.g., from a prior iteration of training). Stated another way, the recursive inference model may iterate on Yₜ₋₁ 1112, making improvements to the shape and/or structure of the generated representation Yₜ 1126. The generated probability distribution of an augmented latent feature codes Yₜ 1126 may be sampled to generate (or modify) 3D oral care representations described herein.

During training or deployment, a 3D oral care representation 1100 (e.g., a tooth mesh, an appliance or appliance component mesh, a fixture model mesh, one or more transforms, a set of mesh element labels, a coordinate axis, or others disclosed herein) may be provided to the initialization method and be encoded into a latent embedding or latent form. Such inputs 1100 may alternatively be described by 3D point clouds, 3D polylines, 3D voxelized representations, or by other data structures described herein. In some implementations, Yₜ₋₁ may be initialized (e.g., at t==0) 1106 using one or more neural networks 1104 which have been trained to generate latent feature embeddings, such as a transformer encoder or an autoencoder encoder (e.g., P-VQ-VAE), as seen in the initialization method of FIG. 11. Such an encoder may be trained as a part of a reconstruction autoencoder, may be trained independently, or may be trained end-to-end with other neural networks. In some implementations, such a neural network (e.g., an autoencoder or transformer) may take as input one or more mesh element features (e.g., comprising a mesh element feature vector associated with each mesh element in the inputted 3D mesh, 3D point cloud or voxelized representation). Such mesh element features may be generated by a mesh element feature module 1102. Such mesh element features may improve the ability of the one or more neural networks to encode aspects of the shape and/or structure of the inputted 3D representation. The result of this initialization operation is an augmented latent feature code Yₜ₌₌₀ (e.g., augmented by the addition of the mesh element features).

The concatenated AFGₜ 1120 and Yₜ₋₁ 1112 may subsequently be provided to a generative neural network module (e.g., which may contain one or more transformers, one or more autoencoders, or one or more fully connected layers), which may output an updated augmented feature grid distribution Yₜ 1126 for the current timestep of training. The generative neural network module may, in some implementations, contain one or more residual connections between neural network layers, which may assist in model convergence during training. Yₜ 1126 may encapsule at least some aspects of the shape and/or structure of the training dataset. The training process may output Yₜ 1126 for use in a deployment system.

In the deployment method of FIG. 11, aspects of a 3D oral care representation may be sampled from the trained probability distribution of augmented latent feature codes Yₜ 1126 (e.g., values corresponding to a transform may be sampled, values corresponding to a vector may be sampled, points of a point cloud corresponding to that 3D oral care representation may be sampled or the like). In deployment, one or more textual inputs or one or more oral care arguments may be encoded into latent form by a text transformer (e.g., BERT). Such inputs may be projected or reorganized into an augmented feature grid AFG_{I} 1120. AFG_{I} 1120 may be concatenated (or otherwise combined) with Yₜ₌₌₀ 1106, an initial version of the 3D oral care representation which is to be generated (or modified). The concatenated AFG_{I}+Y₀ may pass through the generator module 1124, yielding Y₁, the most-up-to-date version of the 3D oral care representation which is to be generated (or modified). This process may repeat in a recursive fashion or in an iterative fashion, continually refining the augmented latent feature code distribution Yₜ 1126. After this process of refining the augmented latent feature code distribution is complete, a 3D oral care representation may be generated by sampling mesh elements (e.g., points) from Yₜ 1126. The generated (or modified) 3D oral care representation may be outputted for use in clinical treatment (e.g., for the generation of an oral care appliance).

In some implementations, the second ML module may contain one or more generative transformer models. In some implementations, a generative transformer model may be trained to generate transforms for 3D oral care representations such as 3D representations of teeth, appliances, appliance components and the like. In some implementations, a generative transformer model may be trained to generate (or modify) the geometries of 3D oral care representations such as 3D representations of teeth (or other aspects of dentition), appliances, appliance components or the like. A generative transformer model may include one or more transformers, or portions of transformers (e.g., individual transformer encoders or individual transformer decoders). A generative transformer model may include one or more hierarchical feature extraction modules (e.g., modules which extract global, intermediate or local neural network features from a 3D representation—such as a point cloud). Examples of hierarchical neural network feature extraction modules (HNNFEM) include 3D SWIN Transformer architectures, U-Nets or pyramid encoder-decoders, among others. A 3D SWIN Transformer may extract hierarchical neural network features from a 3D representation through a series of consecutive stages of decreasing resolution. The input 3D representation may first undergo (optional) voxelization, and then be encoded in a latent representation. The latent representation may be provided to one or more Swin3D blocks, which may extract hierarchical features from the latent representation. At the top-level (stage 1), the hierarchical features are local features. The latent representation may be provided to stage 2, which may downsample the latent representation, and provide the downsampled latent representation to one or more Swin3D blocks. The resulting hierarchical features are now slightly more global than the features that were extracted in stage 1. Execution flows through stages 3, 4, 5, etc., until the global-most hierarchical neural network features are extracted. The 3D SWIN transformer structure then outputs the accumulated hierarchical neural network features from the several stages.

A HNNFEM may be trained to generate multi-scale voxel (or point) embeddings of a 3D representation (or multi-scale embeddings of other mesh elements described herein). For example, a HNNFEM of one or more layers (or levels) may be trained on 3D representations of patient dentitions to generate neural network feature embeddings which encompass global, intermediate or local aspects of the 3D representation of the patient's dentition.

In some implementations, such embeddings may then be passed to a decoder block, which may be trained to generate transforms for 3D representations of teeth or 3D representations of appliance components (e.g., transforms to place teeth into setups poses, or to place appliances, appliance components, fixture model components or other geometries relative to aspects of the patient's dentition). Stated another way, a HNNFEM may be trained (on 3D representations of patient dentitions or 3D representations of appliances, appliance components or fixture model components) to operate as a multiscale feature embedding network. The decoder block may, in some implementations, unite the multi-scale features before the transforms are predicted (e.g., by concatenation). This consideration of multi-scale neural network features may enable small interactions between aspects of the patient's dentition (e.g., local features) to be considered during the setups prediction, during 3D representation generation or during 3D representation modification. For example, during setups prediction, collisions between teeth may be considered by the setups prediction model, and the model may be trained to minimize such collisions (e.g., by learning the distribution of a training dataset of orthodontic setups that contains few or no collisions). This consideration of multi-scale neural network features may further enable the whole tooth shape (e.g., global features) to be considered during final setups transform prediction. A HNNFEM may, in some implementations, contain ‘skip connections’, as are found in some U-NETS. In some implementations, neural network weights for the techniques of this disclosure may be pre-trained on other datasets, such as 3D indoor room segmentation datasets. Such pre-trained weights may be used via transfer learning, to fine-tune a HNNFEM which has been trained to extract local/intermediate/global neural network features from 3D representations of patient dentitions. A HNNFEM (e.g., which has been trained on 3D representations of patient dentitions, appliance components, or fixture model components) may entail an important technical improvement over other techniques, in that the HNNFEM may enable memory-efficient self-attention operations to be computed on sparse voxels. Such an operation is very important when the 3D representations which are provided at the input contain large quantities of mesh elements (e.g., large quantities of points, voxels, vertices/face/edges).

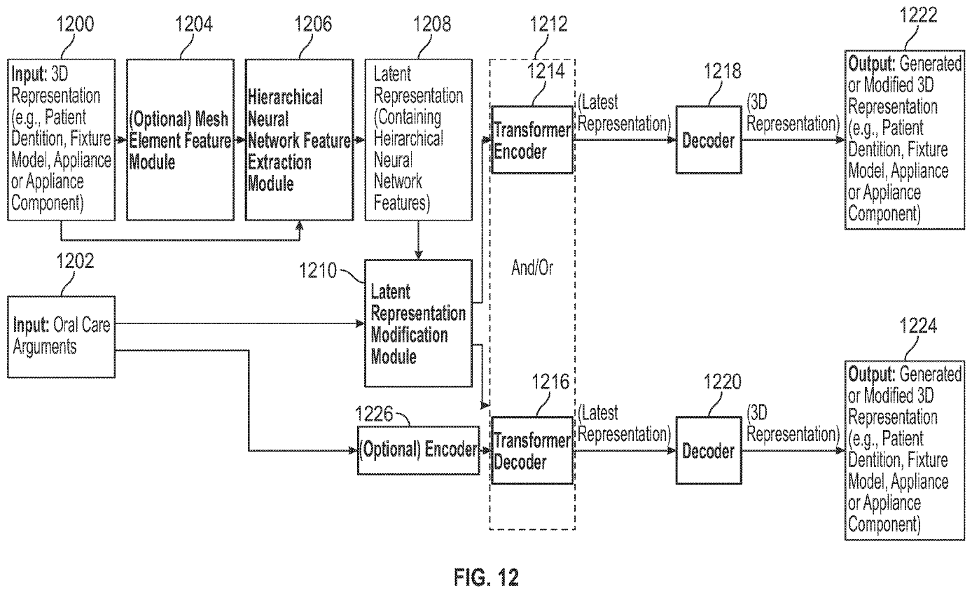

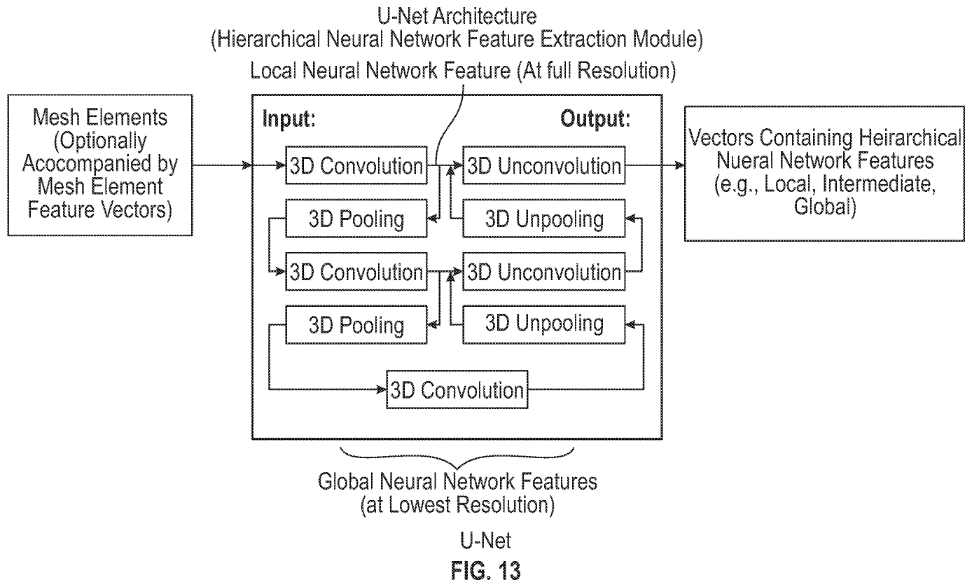

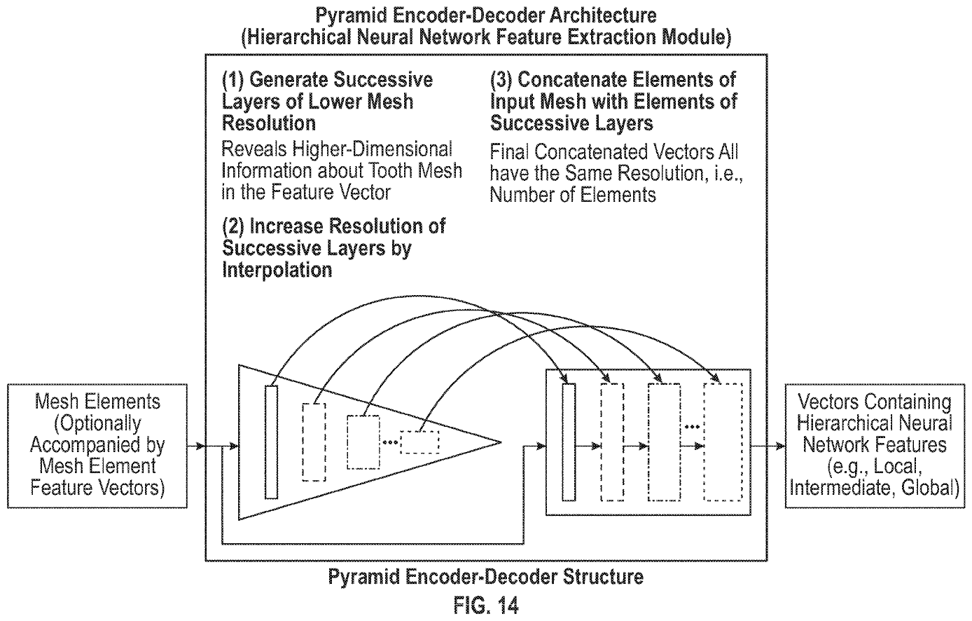

In some implementations, a HNNFEM may be trained for 3D representation generation (e.g., to generate voxels or point clouds which describe aspects of patient dentitions or oral care appliance or oral care appliance components) or for 3D representation modification. For example, a point cloud (or 3D mesh or 3D voxelized representation) generation model may include one or more HNNFEM. The HNNFEM (e.g., which may, in some implementations, function as a type of encoder) may be trained to generate a latent representation (or latent vector or latent embedding) of a 3D representation of the patient's dentition (or of an appliance component or fixture model component). The HNNFEM may be trained to generate hierarchical neural network features (e.g., local, intermediate or global neural network features) of the 3D representation of the patient's dentition (or of an appliance or appliance component). In other implementations, either a U-Net (shown in FIG. 13) or a pyramid encoder-decoder structure (shown in FIG. 14) may be trained to extract hierarchical neural network features. In some implementations, the latent representation may contain one or more of such local, intermediate, or global neural network features. Such a point cloud generation model may, in some implementations, contain a decoder (or ‘upscaling’ block) which may reconstruct the input 3D representation from that latent representation. A HNNFEM 1206 (e.g., as shown in FIG. 12) may have a symmetrical/mirrored arrangement, as may also appear in a UNET. The transformer decoder (or transformer encoder) may be trained to encode sequential or mutually-dependent aspects of the patient's dentition (e.g., set of teeth and gums). Stated another way, the pose of one tooth may be dependent on the pose of surrounding teeth. For example, the generative transformer model may learn dependencies between teeth or may be trained to minimize collisions (e.g., through the use of training by backpropagation as influenced by loss calculation, such as L1, L2, mean squared error (MSE), or cross entropy loss, among others). It may be beneficial for an ML model to account for the sequential or mutually-dependent aspects of the patient's dentition during setups prediction, tooth restoration design generation, fixture model generation, appliance component generation (or placement), to name a few examples. In some implementations, the output of the transformer decoder (or transformer encoder) may be reconstructed into a 3D representation (e.g., a 3D point cloud or 3D voxelized geometry). In some implementations, the latent space output of the transformer decoder (or transformer encoder) may be sampled, to generate points (or voxels). The latent representation which is generated by the transformer decoder (or transformer encoder) may be provided to a decoder. This latter decoder may perform one or more of a deconvolution operation, an upscaling operation, a decompression operation, or a reconstruction operation, among others.

A 3D representation 1200 (e.g., a 3D representation of the patient's dentition, a fixture model component which is to be modified, an appliance component which is to be modified, or the like) may be provided to an optional mesh element feature module 1204, which may provide its output to a HNNFEM 1206. The latent representation 1208 which is generated by the HNNFEM may contain local, global or intermediate neural network features of the 3D representation 1200, among other data. The latent representation 1208 and/or oral care argument 1202 may be provided to latent representation modification module (LRMM) 1210. Positional information (or order information) may, in some implementation, be concatenated with the latent representation that is generated by LRMM 1210. The output of the concatenation may be provided to a transformer module 1212. The output of the concatenation may be provided to either the transformer decoder 1216 or the transformer encoder 1214, allowing the transformer structures to learn positional relationships associated with aspects of the 3D representation (e.g., the order of teeth in an arch, or the order of numerical elements in a latent vector). Oral care arguments 1202 may undergo optional encoding (1226), and subsequently be provided to the transformer decoder 1216. The transformer decoder 1216 may have multi-headed attention. The transformer decoder 1216 may generate a latent representation, which may be reconstructed (1220) into a 3D representation, which may be sent to the output (1224). Likewise, the transformer encoder 1214 may generate a latent representation, which may be reconstructed (1218) into a 3D representation, which may be sent to the output (1222). The transformer decoder may include one or more feed-forward layers. Some non-limiting implementations of the transformer decoder may be 500 MB-2 GB in size. Positional information may be concatenated (or otherwise combined) with the latent representation of the received input data. This positional information may improve the accuracy of processing an arch of teeth, each of which may occupy a well-defined sequential position in the arch. For example, positional information can ensure that teeth appear in an order in the arch which is anatomically plausible. Stated another way, predictions that are anatomically unlikely are avoided by such a model.

The transformer decoders (or transformer encoders) of this disclosure may enable multi-headed attention, meaning that the transformers ‘attend jointly’ to different portions of the input data (e.g., multiple teeth in an orthodontic arch, or multiple cliques of mesh elements in a 3D representation). Stated another way, multi-headed attention may enable the transformer to simultaneously process multiple aspects of the 3D oral care representation which is undergoing processing or analysis. Because the presence of multiple heads (e.g., neural network modules) may enable for multiple attention computations, the transformer may capture and successfully account for complex dependencies between teeth (e.g., in an orthodontic setup prediction) or between mesh elements (e.g., during 3D representation generation or modification). These multiple attention heads enable the transformer to learn long and short-range information from any portion of the received 3D oral care representation, to any other portion of the received 3D oral care representation that was provided to the input of the transformer. Furthermore, during model training, using multiple attention heads may enable the transformer model to extract or encoder different neural network features (or dependencies) into the weights (or bias) of each attention head.

The decoders 1220 or 1218 may use one or more deconvolutional layers (aka inverse convolution) to reconstruct a latent representation into a 3D representation (e.g., point cloud, mesh, voxels, etc.). The decoder may include one or more convolution layers. The decoder may include one or more sparse convolution/deconvolution layers (e.g., as enabled by the Minkowski framework). The decoder may function in manner which is agnostic of sequence (e.g., the order of teeth in an arch or the order of numerical elements in a latent vector). Some non-limiting implementations of the decoder may be 100 MB-200 MB in size.

The generative transformer model may be trained to perform a reparameterization trick in conjunction with the latent representation, such as may also be performed by a variational autoencoder (VAE). Such an architecture may enable modifications to be made to the latent representation (e.g., based on the instructions contained within oral care arguments) to generate a 3D oral care representation (e.g., a tooth restoration design, a fixture model, an appliance component or others disclosed herein) which meets the clinical treatment needs of the patient. Such a generated 3D oral care representation may then be used in the generation of an oral care appliance (e.g., such as in a clinical context where the patient waits in the doctor's office in between intra-oral scanning and 3D printing of an appliance).

A HNNFEM may, in some implementations, be trained to segment a 3D representation (e.g., a point cloud, 3D mesh or voxelized representation) of the patient's dentition. A HNNFEM may generate one or more mesh element labels for one or more mesh elements of such a 3D representation. The mesh element labels may be used to segment aspects of a 3D representation of the patient's dentition (e.g., segmenting the individual teeth or the gums) or to perform mesh cleanup operations on 3D representations of the patient's dentition. Encoder-decoder structures (e.g., transformers, U-Nets or autoencoders) may down-sample the input 3D representation according to a voxel expansion scaling ratio, to reveal increasingly local neural network features. Examples of such voxel expansion scaling ratios may include, for example, non-linear scalings (e.g., 16,8,4,4,2) or linear scalings (e.g., 32,16,8,4,2), among others. The complimentary up-sampling scaling ratio would then be used by the decoder (e.g., 2,4,4,8,16, or 2,4,8,16,32, and the like). Although this example describes the scaling of voxels, other mesh elements (e.g., points, vertices, edges or faces) may also be down-sampled/up-sampled, in other implementations.

An ML model to generate (or modify) a fixture model may generate (or modify) fixture model components. Such an ML model is called a fixture model generation or modification (FMGM) ML model.

In some implementations, a generative transformer model (as described in relation to FIG. 12) may be trained on a dataset including fixture model component data, to generate (or modify) digital fixture model components or digital fixture models. Oral care arguments may be provided to a generative transformer model, to enable customization of the generated (or modified) fixture models. For example, a generative transformer model may be trained to generate (or modify) a 3D oral care representation, such as a fixture model component (e.g., a digital pontic tooth or others digital representations described herein). In some examples, training dataset of cohort patient case data may contain patient cases with gaps (e.g., gaps or spaces between teeth) which call for the use of digital pontic teeth to fill those gaps. Such a patient case 1200 may have an associated ground truth or reference digital pontic tooth available for each gap (e.g., to use in loss calculation). Optional mesh element features for the patient case 1200 using a mesh element feature module 1204. The patient case data 1200 or the optional associated mesh element features may be received by a hierarchical neural network feature extraction module (HNNFEM) 1206, which may compute a latent representation 1208 of the case data 1200. Such a latent representation 1208 may contain global, intermediate, or local neural network features. Optional oral care arguments 1202 or the latent representation 1208 may be received by a latent representation modification module (LRMM) 1210, which may modify the latent representations 1208 of the patient's dentition in a fashion which is intended to bring about a desired shape and/or structure to the generated (or modified) fixture model component (e.g., a digital pontic tooth). In some implementations, the output of LRMM 1210 is provided to a transformer encoder 1214, which may output a latent representation which may be reconstructed by decoder 1218, yielding one or more generated (or modified) digital pontic teeth 1222. In some implementations, the optional oral care arguments 1202 may be embedded into a latent form by encoder 1226. The optional latent representation of the oral care arguments may be provided to transformer decoder 1216. LRMM 1210 may, in some implementations, modify the latent representation 1208 to manifest a desired shape and/or structure in the generated (or modified) 3D representations 1222 or 1224. LRMM may have been trained to modify latent representations (e.g., latent vectors), based at least in part on oral care arguments which are provided to the LRMM. For example, LRMM may modify a latent representation of a template (or reference) digital pontic tooth design (e.g., which comes from a pre-fab library, and which may be intended to undergo customization to fill a gap in the patient's dentition) based on one or more oral care arguments (e.g., a restoration design metric value, such as “Bilateral Symmetry and/or Ratios”) that are provided to the LRMM. The template (or reference) digital pontic tooth design may be provided to the method as a part of the one or more 3D representations 1200. The one or more oral care arguments may describe some aspects of the intended shape and/or structure of the post-generation digital pontic design. Other aspects of the post-generation digital pontic design may be determined by the transformer decoder 1216 or transformer encoder 1214, based on the patient's dentition 1200. The output of LRMM 1210 may be provided to transformer decoder 1216, which may generate a latent representation. The generated latent representation may be reconstructed using decoder 1220 into a 3D oral care representation which is suitable for use in oral care appliance generation. For example, the generated latent representation may be reconstructed using decoder 1220 into one or more generated (or modified) digital pontic teeth 1224. Either or both of the generated (or modified) digital pontic teeth 1222 or 1224 may be compared to corresponding ground truth or reference digital pontic teeth, to compute one or more loss values. Losses include L1, L2, cross-entropy or other losses described herein. Such loss (or losses) may be used to train (e.g., using backpropagation), at least in part, one or more of HNNFEM 1206, transformer encoder 1214, transformer decoder 1216, decoder 1218 or decoder 1220. In some implementations, a discriminator may be used to train, at least in part, one or more of these modules. Some implementations may execute one of the modules within module 1212 (e.g., transformer encoder 1214 or transformer decoder 1216). Some implementations may execute both of the modules within module 1212.

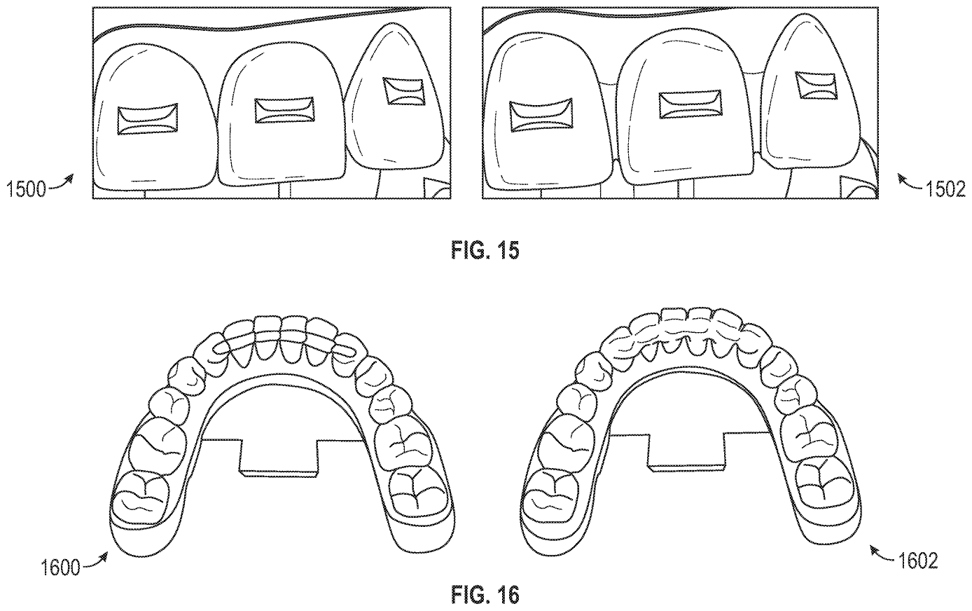

In some implementations, a recursive inference (RI) model (as described in relation to FIG. 11) may be trained on a dataset including fixture model component data, to generate (or modify) digital fixture model components or digital fixture models. Oral care arguments may be provided to an RI model, to enable customization of the generated (or modified) fixture models. For example, an RI model may be trained to generate interproximal webbing, in which the input data 1100 may comprise the patient's segmented teeth (e.g., as shown in the teeth without interproximal webbing 1500 in FIG. 15). In some implementations, an RI model may be trained to modify existing interproximal webbing, in which case the input data 1100 may comprise patient's segmented teeth with some amount of pre-existing interproximal webbing (e.g., as shown in the teeth with interproximal webbing 1502 in FIG. 15). An example of training data 1100 may comprise at least a set of segmented teeth and have an associated ground truth or reference example of interproximal webbing (e.g., a 3D representation of interproximal webbing, for use in loss calculation). Mesh element feature vectors may be computed for the representations of the patient's dentition 1100. The representations of the patient's dentition and associated (optional) mesh element features may be provided to encoder 1104, which may generate an initial latent representation of the patient's dentition 1106. Oral care arguments 1108 may comprise oral care parameters described herein (e.g., which may contain non-text instructions to the generator module 1124 regarding intended aspects of the interproximal webbing which is to be generated or modified) or oral care metrics described herein (e.g., which may measure or quantify aspects of the patient's dentition—such as quantifying the amount of interproximal webbing, percentage of interproximal volume that is occupied by the webbing, smoothness of the webbing, surface curvature of the webbing, count of interproximal spaces with webbing, percentage of interproximal spaces with webbing, or the like).

When training the RI model, the oral care metrics 1108 may quantify or measure aspects of the particular example of data being used for training 1100. When the RI model is in deployment, the oral care arguments 1108 (e.g., oral care metrics or oral care parameters) may specify intended aspects of the interproximal webbing which is intended to be imparted to the patient's dentition 1100. The oral care arguments 1108 may be provided to an optional ML model 1134, such as a neural network, which may generate one or more latent representations (or embeddings) of the oral care arguments 1108. In some implementations, text-based oral care parameters (e.g., a textual description of an intended interproximal webbing which is to be generated or modified) 1110 may be provided to a text transformer (e.g., a BERT-based transformer), which may generate a latent representation 1116 of those text-based oral care parameter. These latent representations may be concatenated (or otherwise reformatted) 1118 into augmented feature grid AFGₜ 1120. The augmented feature grid AFGₜ 1120 may be concatenated (or otherwise combined) with the current iteration of the latent representation of the object which is being generated 1112 (e.g., the initialized representation 1106 of the input data 1100). The result of the concatenation may be provided to the generator module 1124, which may contain one or more generative neural networks. The generator module 1124 may output an updated latent representation 1126 of the object which is being generated (e.g., the patient's dentition with interproximal webbing under construction). This updated latent representation 1126 may be sent back 1130 to the input pathway 1112, so that the method may run again. After several iterations of refinement, the generated (or modified) latent representation of the patient's dentition with interproximal webbing 1126 may be outputted. The latent representation 1126 may, in some implementations, be reconstructed (e.g., using a decoder 1128) into a 3D representation of the patient's dentition with interproximal webbing applied 1132. The latent representation 1126 may, in some implementations, be sampled to generate mesh elements (e.g., points, voxels or vertices) of a 3D representation of the patient's dentition with interproximal webbing applied 1132. In some implementations, the generator module 1124 may be trained, at least in part, by computing a loss and then executing backpropagation. Such a loss may quantify the difference between a predicted output data 1132 and a corresponding ground truth or reference example of the output data. Losses include L1, L2, cross-entropy, or others disclosed herein. In some implementations, the generator module 1124 may be trained, at least in part, by a discriminator.

In other implementations, an RI model may be trained on other 3D oral care representations 1100 described herein (e.g., tooth restoration design, appliance components, other fixture model components, mesh element labels, and the like), to generate (or modify) respective 3D oral care representations 1132. Other such implementations may condition on oral care metrics described herein, or the implementations may condition on oral care metrics which are customized to aspects of the 3D oral care representation used in training. For example, “Length and/or Width” or “Height of Contour” (among others described herein) may be used for a pontic tooth design generation or a tooth restoration design generation. “Bilateral Symmetry and/or Ratios” (among others) may be used for appliance component generation (e.g., the generation of a parting surface for a dental restoration appliance).

Information pertaining to an intended type of material (e.g., material to be used in thermoforming orthodontic aligner trays) may, in some implementations, be provided to the FMGM model. For example, the shape and/or size of blockout which is generated may be based, at least in part, on the information pertaining to an intended material. Information pertaining to tooth type, to a region of an arch (e.g., anterior or posterior) or to aspects of one or more individual interproximal areas may, in some implementations, also be provided to the FMGM model. The benefit of these inputs is that the generated (or modified) fixture model component may be customized to the treatment needs of the patient.

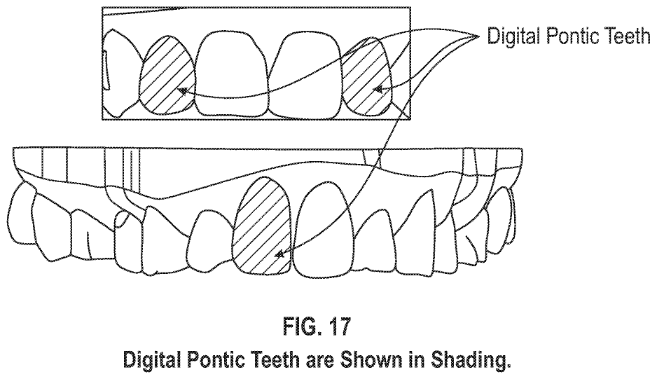

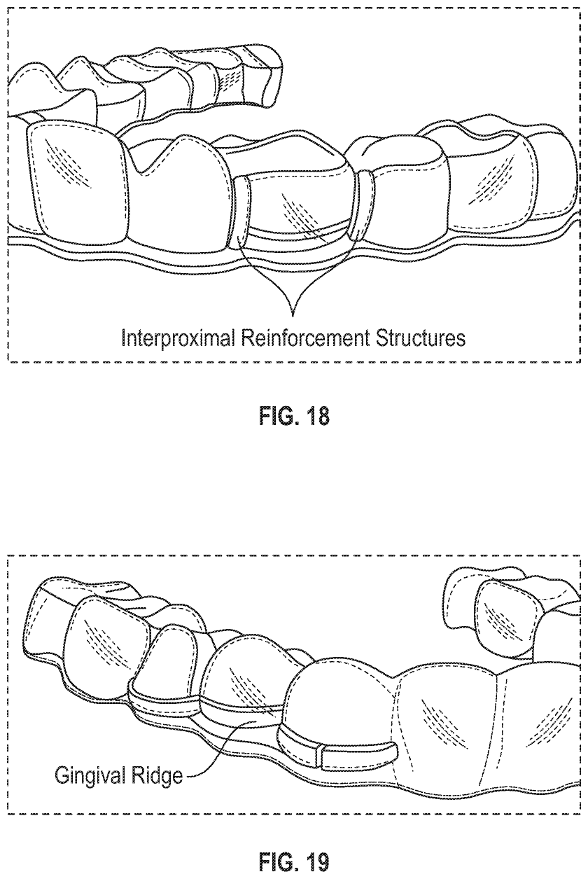

A digital fixture model may comprise 3D representations of the patient's dentition, with optional fixture model components attached to that dentition. 3D representation generation techniques of this disclosure (e.g., techniques to generate 3D point clouds or 3D voxelized representations) may be trained to generate (or modify) aspects of a digital fixture model, to include treatment-enhancing fixture model components. Fixture model components may include 3D representations (e.g., 3D point clouds, 3D meshes, or voxelized representations) of one or more of the following non-limiting items: 1) interproximal webbing—which may fill-in space or smooth-out the gaps between teeth to ensure aligner removability (see, e.g., FIG. 15 and related description). 2) blockout—which may be added to the fixture model to remove overhangs that might interfere with plastic tray thermoforming or to ensure aligner removability (see, e.g., FIG. 16 and related description). 3) bite blocks—occlusal features on the molars or premolars intended to prop the bite open. 4) bite ramps—lingual features on incisors and cuspids intended to prop the bite open. 5) interproximal reinforcement—a structure on the exterior of an oral care appliance (e.g., an aligner tray), which may extend from a first gingival edge of the appliance body on a labial side of the appliance body along an interproximal region between the first tooth and the second tooth to a second gingival edge of the appliance body on a lingual side of the appliance body. The effect of the interproximal reinforcement on the appliance body at the interproximal region may be stiffer than a labial face and a lingual face of the first shell. This may allow the aligner to grasp the teeth on either side of the reinforcement more firmly (see, e.g., FIG. 18). 6) gingival ridge structure—a structure which may extend along the gingival edge of a tooth in the mesial-distal direction for the purpose of enhancing engagement between the aligner and a given tooth (see, e.g., FIG. 19). 7) torque points—structures which may enhance force delivered to a given tooth at specified locations. 8) power ridges—structures which may enhance force delivered to a given tooth at a specified location. 9) dimples—structures which may enhance force delivered to a given tooth at specified locations. 10) digital pontic tooth—structure which may hold space open or reserve space in an arch for a tooth which is partially erupted, or the like. In aligners, a physical pontic is a tooth pocket that does not cover a tooth when the aligner is installed on the teeth. The tooth pocket may be filled with tooth-colored wax, silicone, or composite to provide a more aesthetic appearance. Examples of digital pontic teeth are shown in FIG. 17 (see shaded teeth). 11) power bars—blockout added in an edentulous space to provide strength and support to the tray. A power bar may fill-in voids. Abutments or healing caps may be blocked-out with a power bar. 12) trim line—digital path along the digital fixture model, which may approximately follow the contours of the gingiva (e.g., may be biased 1 or 2 mm in the gingival direction). The trimline may define the path along which a clear aligner may be cut or separated from a physical fixture model, after 3D printing. 13) undercut fill—material which is added to the fixture model to avoid the formation of cavities between the fixture model's height of contour and another boundary (e.g., the gingiva or the plane that the plane that undergirds the physical fixture model after 3D printing).

Techniques of this disclosure may also generate (or modify) other geometries that intrude on the tooth pocket or reinforce an oral care appliance (e.g., an orthodontic aligner tray). Techniques of this disclosure may, in some implementations, determine the location, size, and/or shape of a fixture model component to produce a desired treatment outcome.

Interproximal webbing may be generated using techniques of this disclosure, for example during the fixture model quality control phase of orthodontic aligner fabrication. Interproximal webbing is material (e.g., which may comprise mesh elements, such as vertices/edges/faces, among others) that smooths-out or creates a positive fill in the interproximal areas of the fixture model. Interproximal webbing is extra material added to the interproximal areas of the teeth in a digital fixture model (e.g., which may be 3D printed and rendering in physical form) to reduce the tendency of the aligner, retainer, attachment template, or bonding tray to lock onto the physical fixture model during the orthodontic aligner thermoforming process. Interproximal webbing may improve the functioning of the aligner tray by improving the ability of the tray to slide off of the fixture model after thermoforming, or by improving the fit of the tray onto the patient's teeth (e.g., making the tray easier to insert onto the teeth or to remove from the teeth).

Blockout (e.g., which may comprise mesh elements, such as vertices/edges/faces, among others) may be generated using techniques of this disclosure. Blockout is material which may be added to undercut regions of a digital fixture model, so that the aligner, retainer, attachment template, or bonding tray, 3D printed mold, or other oral care appliance, from locking onto the physical fixture model during the orthodontic aligner thermoforming process. Block out may be generated to fill-in a portion of a digital fixture model with an undercut, so that a thermoformed aligner tray does not grab onto that undercut. Blockout may improve the functioning of the aligner tray by improving the ability of the tray to slide off of the fixture model after thermoforming.

In the arch 1600 of FIG. 16, an intraoral scan of the patient's teeth includes a lingual retainer on the lingual portion of the anterior teeth. In the arch 1602 of FIG. 16, blockout is applied to remove any undercuts beneath the lingual retainer, and to fill narrow interproximal spaces that may result from tooth segmentation of an arch that contains a lingual retainer. The blockout may facilitate later thermoforming (e.g., help avoid the aligner tray getting stuck on the physical fixture model).

Pontic tooth designs may be generated using techniques of this disclosure. A pontic tooth is a digital 3D representation of a tooth which may act as a placeholder in an arch. In an aligner, a pontic may function as a tooth pocket that may be filled with a tooth-colored material (e.g., wax) to improve aesthetics. A pontic tooth may hold a space in the arch open during orthodontic setups generation. When automated setups prediction is performed, transforms for intermediate stages may be generated. One or more pontic teeth may be defined to hold open space for missing teeth or act as a placeholder as space closes or opens in the arch during setups predicted staging or an unerupted tooth may erupt into a space where a pontic is present. Creating a pocket for the tooth to erupt into.

Digital pontic teeth may be placed in (or generated within) an arch (e.g., during setups generation or fixture model generation) to reserve space in an arch for missing or extracted teeth (e.g., so that adjacent teeth do not encroach upon that space over the course of successive intermediate stages of orthodontic treatment). In some implementations, digital pontic tooth may be used when the space (e.g., the space that is to be held open) is at least a threshold dimension (e.g., a width of 4 mm, among others). In some instances, digital pontic teeth for UL4-UR4 or LL4-LR4 may be placed (or generated or modified) when space is available or when there is a partially erupted tooth within the space. When there is a partially erupted tooth in the space, a digital pontic tooth may be placed over the erupting tooth to maintain a space for the erupting tooth to erupt into. Pontic teeth may be placed (or generated or modified) to be inside the gingiva, to minimize (or avoid) heavy occlusal contacts (e.g., contacts between the chewing surfaces of the upper or lower arches), or to cover an erupting tooth (when present), among other conditions.

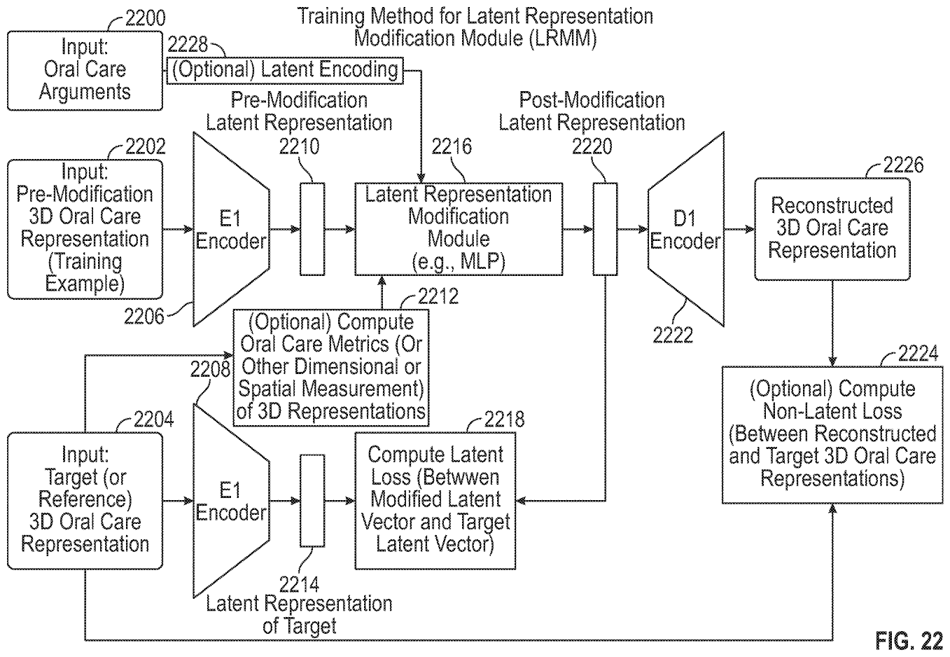

Encoder E1 2206 of FIG. 22 may be trained to generate a pre-modification latent representation 2210 for the pre-modification 3D oral care representation 2202 (e.g., a 3D representation such as teeth, or other types of 3D oral care representations). 3D oral care representations which are 3D representations may comprise one or more mesh elements (as described herein). Encoder E1 2206 may, in some implementations, use a mesh element feature module (as described elsewhere in this specification) to compute mesh element feature vectors for one or more of the mesh elements. For example, these mesh element features may assist the encoder E1 2206 in encoding the shape and/or structure of the patient's dentition, resulting in representations that are more accurate (e.g., representations which may, in some implementations, be reconstructed into facsimiles of the original teeth or gums). Such representations (e.g., latent representations or latent forms) may comprise information-rich or reduced-dimensionality versions of the provided teeth (or gums) from the patient case. In other implementations, the encoder E1 2206 may be replaced by other latent representation generation ML modules (e.g., one or more U-Nets, one or more transformer encoders, one or more pyramid encoder-decoders, one or more other neural network modules (e.g., pairs of convolution and pooling layers), or other representation-generation models described herein).

Representation learning techniques may be used to train a machine learning model (e.g., a latent representation modification module—LRMM) to modify a latent representation of a 3D oral care representation in a manner that, when the latent representation is reconstructed (e.g., using a decoder), the reconstructed 3D oral care representation includes properties (e.g., shape, structure, etc.) which make that 3D oral care representation suitable for use in generating an oral care appliance. A reconstruction autoencoder (e.g., a variational autoencoder with optional continuous normalizing flows) may be trained to reconstruct 3D oral care representations from a training dataset 2202. The encoder 2206 may be trained to encode the training data 2202 into a latent representation, and then the decoder may be trained to reconstruct that latent representation into a close facsimile 2226 of the original training data 2202. The result is a trained encoder-decoder structure. The latent representation in between the encoder 2206 and decoder 2222 may undergo modification by an LRMM, so that the reconstructed 3D oral care representation 2226 contains modifications relative to the original 3D oral care representation 2202.

In some implementations, the LRMM may be trained to modify latent representations of data from cohort patient cases, such patient dentition (e.g., the patient's teeth). The training dataset may contain pairs of pre-modification data 2202, post-modification (or target) data 2204.

In some instances, the training data may include one or more 3D representations of the patient's pre-modification dentition 2202 (e.g., the patient's pre-restoration teeth), and corresponding 3D representations of the patient's post-modification (or target) dentition 2204 (e.g., the patient's post-restoration teeth).

In some instances, the training data may include one or more 3D representations of a pre-modification oral care appliance component 2202 (e.g., prefab library components or generated components—such as parting surfaces, etc.), and one or more corresponding 3D representations of post-modification (or target) oral care appliance components 2204.

In some instances, the training data may include one or more pre-modification dentition and/or pre-modification fixture model components 2202 (e.g., a digital pontic tooth, blockout, interproximal webbing, or others described herein), and one or more corresponding 3D representations of the patient's post-modification (or target) dentition and/or post-modification (or target) fixture model components 2204 (e.g., the patient's dentition with interproximal webbing added to the interproximal spaces between some teeth—such as anterior teeth).

In some instances, the training data may include one or more pre-modification transforms 2202, such as transforms to place teeth (or appliance components or fixture model components) into poses which are suitable for oral care appliance generation, and one or more corresponding post-modification (or target) transforms 2204.

In some instances, the training data may include one or more pre-modification mesh element labels (e.g., labels which may be used in mesh segmentation or mesh cleanup), which may be accompanied by one or more corresponding post-modification (or target) mesh element labels.

During the training of the LRMM 2216, a 3D oral care representation of training data 2202 (e.g., pre-modification data) may have a corresponding 3D oral care representation of target data (e.g., post modification data) 2204. LRMM 2216 may contain one or more MLPs, or one or more U-Nets (among other of the architectures disclosed herein). In some implementations, the encoder E1 2206, the decoder D1 2222 and the LRMM 2216 may be trained end-to-end. In other implementations, the encoder E1 2206, the decoder D1 2222 may be trained separately from the LRMM 2216.

The training data 2202 may undergo latent encoding (e.g., using encoder 2206), which may generate pre-modification latent representation 2210. The corresponding target data 2204 may likewise undergo latent encoding (e.g., using encoder E1 2208), which may generate a latent representation of the target data 2214. The encoder 2208 may, in some implementations, be the same as encoder 2206. In some implementations, the pre-modification latent representation 2210 may be provided to LRMM 2216. In some implementations, oral care arguments 2200 may be provided to the LRMM 2216. In some implementations, oral care arguments 2200 may undergo latent encoding (2228), before being provided to LRMM 2216. In some implementations, the latent encoding (2228) may use an encoder to encode categorical, Boolean or real valued oral care arguments 2200. In some implementations, the latent encoding (2228) may use a CLIP encoder (or a GPT transformer encoder or GPT transformer decoder) to generate latent representation for textual oral care arguments 2200 (e.g., textual descriptions of modifications which are to be performed).

In some implementations, (optional) oral care metrics (or other dimensional measurements or calculations) may be computed (2212) on the target data 2204, and subsequently be provided to LRMM 2216. These oral care metrics (or other dimensional measurements) may specify to the LRMM 2216 aspects of the target shape and/or structure for the reconstructed 3D oral care representation 2226. The LRMM may be trained to generate a post-modification latent representation 2220, which may be provided to decoder 2222, which may generate a reconstructed 3D oral care representation 2226 with a shape, structure, dimensions, numerical values, or other values which are suitable for use in oral care appliance generation.

The LRMM 2216 may be trained, at least in part, by a latent loss function (e.g., for comparing latent vectors—such as cross-entropy or others described herein) or by a non-latent loss function (e.g., for comparing data structures which are in their original, non-latent forms). Examples of non-latent losses include reconstruction loss or KL-Divergence loss (e.g., for 3D representations—such as point clouds, or other data structures described herein), L1 or L2 losses (e.g., for transforms, or other data structures described herein), cross-entropy loss (e.g., for mesh element labels, or other data structures described herein), or others described herein. The latent loss may be computed (2218) between the latent representation of the target data 2214, and the generated post-modification latent representation 2220. The non-latent loss may be computed (2224) between the target 3D oral care representation 2204, and the reconstructed 3D oral care representation 2226.

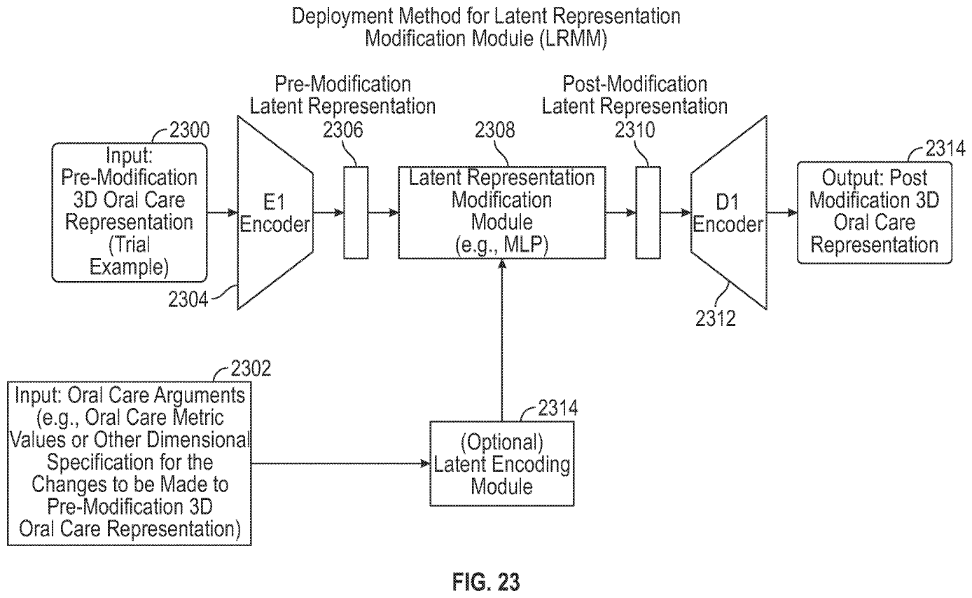

In deployment, the input 2300 of FIG. 23 is not intended to represent training data, but rather specifies one or more 3D oral care representations which are to be modified (e.g., a pre-restoration tooth mesh, a mold parting surface to be modified, a patient dentition which is to receive interproximal webbing or blockout, or others described herein) may be provided to encoder E1 2304. The encoder 2304 may generate a pre-modification latent representation 2306. The pre-modification latent representation 2210 may be provided to the LRMM 2308, which may generate a post-modification latent representation 2310. Post-modification latent representation 2310 may be provided to decoder D1 2312, which may reconstruct post-modification latent representation 2310 into post-modification 3D oral care representation 2314. In some implementations, oral care arguments 2302 may be provided to the LRMM 2308, providing specification for the intended modification(s) which is to be applied to latent representation 2306. Such oral care arguments may include, for example, specifications for the shape and/or structure of an intended post-modification 3D oral care representation 2314 (e.g., dimensional measurements that describe the intended shape and/or structure). In some implementations, one or more oral care metrics, which may measure (or quantify aspects of) the shape and/or structure of an intended 3D oral care representation (e.g., a 3D representation of a tooth, a fixture model component, an appliance component, set of orthodontic setup transforms, or other 3D representation which is to be modified), may be provided to the LRMM 2308. In some instances, oral care arguments 2302 may contain text, which may undergo latent encoding (2314) (e.g., using a CLIP text encoder, or a GPT transformer encoder), before being provided to the LRMM 2308.

Neural network-based techniques are described herein for the placement of oral care articles in relation to one or more 3D representations of teeth or other oral care articles. Oral care articles which are to be placed may include: dental restoration appliance components, oral care hardware (e.g., a lingual bracket, a labial bracket, an orthodontic attachment, a bite ramp, etc.), fixture model components, or the like. Furthermore, neural network-based techniques are described herein for the generation of the geometry and/or structure of oral care articles based, at least in part, on one or more 3D representations of teeth. Oral care articles which may be generated include: a dental restoration appliance component, a dental restoration tooth design, a crown, a veneer, or the like.

### EXAMPLES

Example 1. A method of generating an output three-dimensional (3D) oral care representation for oral care treatment, the method comprising:
receiving, by processing circuitry of a computing device, an input 3D oral care representation;
executing, by the processing circuitry, a representation generation module to generate a reformatted version of the input 3D oral care representation;
executing, by the processing circuitry, a trained generator network that comprises at least a trained transformer model to:
   generate, using the reformatted version of the input 3D oral care representation, an output 3D oral care representation; and
outputting, by the processing circuitry, the output 3D oral care representation.
   Example 2. The method of Example 1, wherein the input 3D oral care representation represents one or more teeth of a dental arch.
   Example 3. The method of Example 1, further comprising providing, by the processing circuitry, as input to at least one of the representation generation module or the trained generator network, one or more oral care parameters.
   Example 4. The method of any of Examples 1-3, wherein the output 3D oral care representation represents a tooth restoration design.
   Example 5. The method of Example 4, wherein the output 3D oral care representation is used in a design of an oral care appliance.
   Example 6. The method of Example 5, wherein the oral care appliance is a dental restoration appliance.
   Example 7. The method of any of Examples 1-3, wherein the output 3D oral care representation represents a generated component for an oral care appliance.
   Example 8. The method of Example 7, wherein the output 3D oral care representation is used in a design of an oral care appliance.
   Example 9. The method of Example 8, wherein the oral care appliance is an orthodontic appliance.
   Example 10. The method of Example 1, wherein the input 3D oral care representation comprises one or more aspects of a patient dentition.
   Example 11. The method of Example 10, wherein the one or more aspects of the patient dentition are indictive of one or more attributes of a tooth in the patient dentition.
   Example 12. The method of Example 1, wherein the input 3D oral care representation comprises a 3D oral care representation generated using an automated process.
   Example 13. The method of Example 1, wherein the input 3D oral care representation comprises a clinician-designed 3D oral care representation.
   Example 14. The method of Example 1, further comprising providing, by the processing circuitry, as input to at least one of the representation generation module or the trained generator network, one or more mesh element features.
   Example 15. The method of Example 1, wherein the representation generation module comprises one or more of an autoencoder, a transformer, a U-Net, a pyramid encoder-decoder, one or more convolutional layers, or one or more pooling layers.
   Example 16. The method of Example 1, wherein the reformatted version of the input 3D oral care representation comprises a reduced-dimensionality version of the input 3D oral care representation.
   Example 17. The method of Example 1, further comprising training a machine learning (ML) model according to a transfer learning paradigm using at least one of the representation generation module or the trained generator network.
   Example 18. The method of Example 1, wherein at least one of the representation generation module or the trained generator network is trained according to a transfer learning paradigm.
   Example 19. A device for modifying a three-dimensional (3D) oral care representation for oral care treatment, the device comprising:
interface hardware configured to receive an input 3D oral care representation;
processing circuitry configured to:
   execute a representation generation module to generate a reformatted version of the input 3D oral care representation;
   execute a trained generator network that comprises at least a trained transformer model to:
      generate, using the reformatted version of the input 3D oral care representation, an output 3D oral care representation; and
a memory unit configured to store the output 3D oral care representation generated by the processing circuitry.
   Example 20. The device of example 19, wherein the device is deployed in a clinical context.

### EXAMPLES

Example 1. A method of modifying an input three-dimensional (3D) oral care representation for oral care treatment, the method comprising:
receiving, by processing circuitry of a computing device, the input 3D oral care representation;
providing, by the processing circuitry, the input 3D oral care representation as an execution-phase input to a trained autoencoder that comprises at least a multi-dimensional encoder and a multi-dimensional decoder;
executing, by the processing circuitry, the multi-dimensional encoder to encode the first 3D oral care representation to form a latent representation;
modifying, by the processing circuitry, the latent representation to form a modified latent representation; and
executing the multi-dimensional decoder to reconstruct the modified latent representation to form an output 3D oral care representation that is a version of the input 3D oral care representation with at least one modification,
wherein the at least one modification comprises at least one of: one or more added mesh elements, one or more removed mesh elements, or one or more transformed mesh elements.
   Example 2. The method of Example 1, wherein at least one modification is associated with an appliance component.
   Example 3. The method of Example 1, wherein at least one modification is associated with an archform.
   Example 4. The method of Example 1, wherein at least one modification is associated with a clear tray aligner (CTA) trimline.
   Example 5. The method of Example 1, wherein the input 3D oral care representation comprises one or more aspects of a patient dentition.
   Example 6. The method of Example 5, wherein the one or more aspects of the patient dentition are indictive of one or more attributes of a tooth in the patient dentition.
   Example 7. The method of Example 1, wherein the output 3D oral care representation is used to generate a design for an oral care appliance.
   Example 8. The method of Example 7, wherein the oral care appliance is a dental restoration appliance.
   Example 9. The method of Example 7, wherein the oral care appliance is an orthodontic appliance.
   Example 10. The method of Example 1, wherein the input 3D oral care representation comprises a template version of a 3D oral care representation.
   Example 11. The method of Example 1, wherein the input 3D oral care representation comprises a 3D oral care representation generated using an automated process.
   Example 12. The method of Example 1, wherein the input 3D oral care representation comprises a clinician-designed 3D oral care representation.
   Example 13. The method of Example 1, further comprising providing, by the processing circuitry, as input to the trained autoencoder network, one or more mesh element features.
   Example 14. The method of Example 13, wherein one or more mesh element features comprise one or more feature vectors.
   Example 15. The method of Example 1, further comprising providing, by the processing circuitry, as input to the trained autoencoder network, one or more one or more oral care parameters.
   Example 16. The method of Example 1, wherein the reformatted version of the input 3D oral care representation comprises a reduced-dimensionality version of the input 3D oral care representation.
   Example 17. The method of Example 1, further comprising training a machine learning (ML) model according to a transfer learning paradigm using the trained autoencoder network.
   Example 18. The method of Example 1, wherein the trained autoencoder network is trained according to a transfer learning paradigm.
   Example 19. The method of Example 1, further comprising forming the one or more transformed mesh elements by modifying at least one of a position or an orientation of at least one of the one or more mesh elements.
   Example 20. A device for modifying an input three-dimensional (3D) oral care representation for oral care treatment, the device comprising:
interface hardware configured to receive the input 3D oral care representation;
processing circuitry configured to:
   provide the input 3D oral care representation as an execution-phase input to a trained autoencoder that comprises at least a multi-dimensional encoder and a multi-dimensional decoder;
   execute the multi-dimensional encoder to encode the first 3D oral care representation to form a latent representation;
   modify the latent representation to form a modified latent representation; and
   execute the multi-dimensional decoder to reconstruct the modified latent representation to form an output 3D oral care representation that is a version of the input 3D oral care representation with at least one modification, wherein the at least one modification comprises at least one of: one or more added mesh elements, one or more removed mesh elements, or one or more transformed mesh elements; and
a memory unit configured to store the output 3D oral care representation.
   Example 21. The device of Example 20, wherein the device is deployed in a clinical context.

### EXAMPLES

Example 1. A method of executing a trained machine learning (ML) model to predict a dental archform, the method comprising:
providing, by processing circuitry of a computing device, as execution-phase input, a three-dimensional (3D) oral care representation of a dental arch of a patient to the trained ML mode; and
executing, by the processing circuitry, the trained ML model to output a predicted archform for the patient.
   Example 2. The method of Example 1, wherein the 3D oral care representation comprises one of a 3D mesh or a voxelized representation.
   Example 3. The method of Example 1, wherein the predicted archform comprises one of a set of control points through which a spline is fit, a polyline comprising one or more vertices and/or one or more edges, or a 3D mesh having at least one aligned aspect with respect to a coordinate axis of at least one tooth represented in the 3D oral care representation provided as the execution-phase input.
   Example 4. The method of Example 1, wherein the predicted archform comprises an approximation of one or more contours of the dental arch.
   Example 5. The method of Example 1, wherein the 3D oral care representation includes representations of one or more teeth in at least one of a maloccluded pose, an intermediate stage pose, or a final setup pose.
   Example 6. The method of Example 1, wherein the predicted archform is used for production of an oral care appliance.
   Example 7. The method of Example 1, wherein the predicted archform comprises one or more 3D meshes.
   Example 8. The method of Example 1, wherein the predicted archform comprises one or more polylines.
   Example 9. The method of Example 1, wherein the predicted archform comprises a set of control points through which a spline is fit.
   Example 10. The method of Example 1, wherein the trained ML model comprises a trained neural network.
   Example 11. The method of Example 1, wherein the trained ML model comprises a trained transformer.
   Example 12. The method of Example 1, wherein the trained ML model comprises a trained autoencoder network.
   Example 13. The method of Example 1, wherein the computing device is deployed in a clinical context, and wherein the method is performed in the clinical context.
   Example 14. The method of Example 1, further comprising providing the predicted archform to a trained setups prediction model configured to output a predicted setup for the patient.
   Example 15. The method of Example 1, wherein the trained ML model is trained, at least in part, using a transfer learning paradigm.
   Example 16. The method of Example 1, wherein the trained ML model is used to train, at least in part, a training-phase ML model according to a transfer learning paradigm.
   Example 17. The method of Example 1, wherein the computing device is deployed in a clinical context, and wherein the method is performed in the clinical context.
   Example 18. A computing device configured to execute a trained machine learning (ML) model to predict a dental archform, the computing device comprising:
interface hardware configured to receive a three-dimensional (3D) oral care representation of a dental arch of a patient;
processing circuitry configured to:
   provide the 3D oral care representation of a dental arch of the patient as execution-phase input to the trained ML model; and
   execute the trained ML model to form a predicted archform for the patient; and
a memory unit configured to store the predicted archform for the patient.
   Example 19. The device of Example 18, wherein the device is deployed in a clinical context.

1. A method of generating a three-dimensional (3D) representation of oral care data for oral care treatment, the method comprising:
receiving, by processing circuitry of a computing device, an input 3D representation of the patient's dentition;
executing, by the processing circuitry, a trained first ML module to encode the input 3D representation of the patient's dentition into a first latent representation having a lower order of dimensionality than the input 3D representation of the patient's dentition;
executing, by the processing circuitry, a trained second ML module that comprises at least one of a trained transformer encoder model, or a trained transformer decoder model to:
generate, using the first latent representation of the input 3D representation of the patient's dentition, a second latent representation;
reconstructing, by the decoder, the second latent representation into a reconstructed 3D oral care representation; and
outputting, by the processing circuitry, the reconstructed 3D representation of oral care data.

2. The method of claim 1, further comprising generating, using the reconstructed 3D representation of oral care data, one or more oral care appliances.

3. The method of claim 1, further comprising:
receiving, by the processing circuitry of a computing device, one or more oral care arguments;
influencing, by the one or more oral care arguments, the generating.

4. The method of claim 1, further comprising:
executing, by the processing circuitry, a trained latent representation modification module (LRMM);
modifying, by the LRMM, one or more aspects of the first latent representation; and
generating, using the one or more modified aspects of the first latent representation, the reconstructed 3D tooth representation.

5. The method of claim 4, further comprising:
receiving, by the processing circuitry of a computing device, the one or more oral care arguments;
influencing, by the one or more oral care arguments, the operation of the LRMM.

6. The method of claim 2, wherein the oral care appliance is a dental restoration appliance.

7. The method of claim 2, wherein the oral care appliance is an orthodontic appliance.

8. The method of claim 1, wherein the reconstructed 3D representation of oral care data is a representation of an appliance component.

9. The method of claim 1, wherein the reconstructed 3D representation of oral care data is a representation of an archform.

10. The method of claim 1, wherein the reconstructed 3D representation of oral care data is a representation of the patient's dentition with at least one generated fixture model component.

11. The method of claim 1, wherein the input 3D representation of oral care data comprises a representation of at least one tooth in a pre-restoration state.

12. The method of claim 11, wherein the reconstructed 3D representation of oral care data is a representation of at least one tooth in a post-restoration state.

13. The method of claim 1, wherein the input 3D representation of the patient's dentition comprises one or more mesh elements, and the method further comprises computing, by the processing circuitry, one or more mesh element features.

14. The method of claim 13, further comprises providing, by the processing circuitry, as input to at least one of the first ML module or the second ML module, the one or more mesh element features.

15. The method of claim 1, wherein the first ML module comprises one or more of an encoder, a U-Net, a pyramid encoder-decoder, 3D SWIN transformer, one or more convolutional layers, or one or more pooling layers.

16. The method of claim 1, further comprising training a machine learning (ML) model according to a transfer learning paradigm using at least one of the trained first ML module or the trained second ML module.

17. The method of claim 1, wherein at least one of the trained first ML module or the trained second ML module is trained according to a transfer learning paradigm.

18. The method of claim 1, wherein the computing device is deployed at a clinical context, and wherein the method is performed in near real-time during an encounter with a patient.

19. The method of claim 15, wherein the trained first ML module is configured to generate one or more hierarchical neural network features, and the method further comprises generating the one or more hierarchical neural network features, based at least in part on one or more aspects of at least one of the shape or the structure of the input 3D representation.

20. A computing device for generating a three-dimensional (3D) representation of oral care data for oral care treatment, the computing device comprising:
interface hardware configured to receive an input three-dimensional (3D) representation of the patient's dentition;
processing circuitry configured to:
execute a trained first ML module to encode the input 3D representation of the patient's dentition into a first latent representation having a lower order of dimensionality than the input 3D representation of the patient's dentition;
execute, by the processing circuitry, a trained second ML module that comprises at least one of a trained transformer encoder model, or a trained transformer decoder model to:
generate, using the first latent representation of the input 3D representation of the patient's dentition, a second latent representation;
reconstruct, by the decoder, the second latent representation into a reconstructed 3D oral care representation; and
output, by the processing circuitry, the reconstructed 3D representation of oral care data.
